(19) United States
(12) Patent Application Publication
(10) Pub. No.: US 2026/0176622 A1
(43) Pub. Date: Jun. 25, 2026

(54) METHODS AND COMPOSITIONS FOR IMMUNE CELL CRISPR SCREENS

(71) Applicants: The General Hospital Corporation, Boston, MA (US); The Broad Institute, Inc., Cambridge, MA (US)

(72) Inventors: Marcela V. Maus, Lexington, MA (US); Robert Manguso, Boston, MA (US); Nelson Knudsen, Boston, MA (US)

(73) Assignees: The General Hospital Corporation, Boston, MA (US); The Broad Institute, Inc., Cambridge, MA (US)

(21) Appl. No.: 18/868,561

(22) PCT Filed: May 23, 2023
(86) PCT No.: PCT/US2023/067350

§ 371 Date: Nov. 22, 2024

Related U.S. Application Data
(60) Provisional application No. 63/344,951, filed on May 23, 2022.
(60) Provisional application No. 63/464,483, filed on May 5, 2023.

(51) Int. Cl. C12N 15/113; C07K 14/725

(52) U.S. Cl. CPC C12N 15/113; C07K 14/7051

(57) ABSTRACT

This application discloses in part methods and compositions for mutating immune cells (e.g., CAR-T cells) and determining mutated immune cell cancer killing efficacy in vivo.

### RELATED APPLICATIONS

This application is a national stage filing under 35 U.S.C. § 371 of international application number PCT/US2023/067350, filed May 23, 2023, which claims the benefit under 35 U.S.C. § 119 (e) to U.S. Provisional Application No. 63/344,951, filed May 23, 2022, entitled “Methods and Compositions for Immune Cell CRISPR Screen”, and to U.S. Provisional Application No. 63/464,483, filed May 5, 2023, entitled “Methods and Compositions for Immune Cell CRISPR Screen”, the entire contents of each of which are incorporated herein by reference.

### REFERENCE TO AN ELECTRONIC SEQUENCE LISTING

The contents of the electronic sequence listing (M105370033US02-SUBSEQ-ARM.xml; Size: 1,878,352 bytes; and Date of Creation: Dec. 19, 2024) are herein incorporated by reference in its entirety.

### BACKGROUND

Cancer therapy has been revolutionized by reprogramming immune cells to comprise chimeric antigen receptor (CARs), which direct the immune cell to the target cancer and induce killing of the cancer cell upon binding. CARs are transmembrane proteins that typically comprise an extracellular portion (e.g., antigen binding domain), a transmembrane domain, and an intracellular binding domain. The antigen binding domain is often, but not always, an antibody that binds to a cancer-associated antigen. Upon binding of the antigen binding domain, the intracellular domain activates the immune cell (e.g., T cell), which may result in the immune cell killing the cancer cells. However, numerous factors can influence the efficacy of a CAR based therapy in killing cancer, including idiosyncratic patient characteristics that result in one type of CAR based therapy having more therapeutic efficacy in some patients versus other patients. Thus, there is a general need for CAR therapeutics that can be personalized to patients in treating specific diseases.

### SUMMARY

In some aspects, the present disclosure describes methods and compositions for performing CAR-immune cell (e.g., CAR-T cell or CAR-NK cell) CRISPR screens (e.g., in humans) to identify immune cell genotypes that enhance CAR immune cancer efficacy. For example, this disclosure describes CRISPR guide RNA polynucleotide libraries designed to target genes that are believed to be involved in CAR-T cell efficacy. Thus, in some embodiments, the CRISPR guide RNA library may be used to produce a library of mutant CAR-T cells, the mutant CAR-T cell library comprising CAR-T cells with many different genotypes, and, e.g., comprising the same CAR. The disclosure is directed, in part, to the discovery of collections of genes the alteration of which may influence CAR-T cell efficacy (e.g., tumor killing and persistence), and the subsequent development of CRISPR guide RNA libraries to target the collections of genes. In some embodiments, one or more of the genotypes of the library of mutant CAR-T cells improves CAR-T cell efficacy (e.g., tumor killing and persistence). Further, these mutations may have different effects in different patients and/or when treating different cancers. In some embodiments, the methods described herein comprise generating a mutated CAR-immune cell library (e.g., mutated CAR-T cell library or a mutated CAR-NK cell library), administering the library to a patient (e.g., a human patient), collecting one or more samples of the CAR-T cell library from the patient, and sequencing the guide RNA polynucleotides of the CAR-T cells to identify which CAR-T gene mutants have increased most in prevalence in the samples as an indicator of CAR-T cell efficacy. In some embodiments, the method further comprises administering to the patient CAR-T cells comprising mutations that increase CAR-T cell prevalence in vivo (as determined using the method above). Thus, in some embodiments, these methods provide a strategy for identifying patient specific and/or cancer specific CAR-T cell genome mutations that enhance cancer killing efficacy in vivo.

The compositions and methods described herein provide advantages over previously existing technologies. Results obtained from experiments performed in model organisms are often not recapitulated when experiments are adapted and repeated in a different organism, e.g., in humans. For example, CD19-28z cytotoxic T cells eradicated B-Cell tumors in mice, but had little effect on cancer in humans. Brentjens et al., Nature medicine 9.3 (2003): 279-286 and Brentjens et al., Blood, The Journal of the American Society of Hematology 118.18 (2011): 4817-4828. In another example, a mesothelin directed CAR-T cell killed cancer in mice, but had little effect on cancer in humans. Carpenito et al., Proceedings of the National Academy of Sciences 106.9 (2009): 3360-3365 and Beatty et al., Gastroenterology 155.1 (2018): 29-32. The compositions and methods of the present disclosure are, in part, designed for use in humans. CAR-immune cell genotypes (e.g., CAR-T cell genotypes or CAR-NK cell genotypes) identified as improving cancer killing efficacy in humans require no adaptation from a potentially non-analogous model organism, and therapeutic effects observed can be directly applied to humans, e.g., the human in whom the screen was performed. The disclosure also provides CAR and CRISPR vectors designed for use in humans, e.g., comprising CAR incorporation markers and CRISPR mutation markers that are compatible for use in humans.

Further, the disclosure provides methods and compositions capable of providing conclusive screen results in human. The disclosure provides strategies for determining the characteristics (e.g., the size) of a guide RNA library that could be used in humans and achieve sufficient representation of the mutated CAR-immune cell library for calculations regarding phenotypes of interest, e.g., cancer killing efficacy (see, e.g., Example 2 below for calculation details). In some embodiments, the number of different gRNA polynucleotides in the gRNA library is determined based on these calculations. In some embodiments, a gRNA library designed for a human CAR-immune cell CRISPR screen (e.g., CAR-T cell screen or CAR-NK cell screen) is not genome-wide. Without wishing to be bound by theory, a genome-wide gRNA library may be too large and comprise too many different gRNA polynucleotides to provide conclusive results in humans, e.g., because genome-wide CRISPR libraries contain so many different mutant immune cells (e.g. mutant CAR-T cells) that they may not be adequately represented in a sample collected from the patient, resulting in poor statistical resolution and an inability to identify mutations that contribute to a phenotype, e.g., CAR-T cell efficacy.

This disclosure provides, in part, selected sets of genes to be targeted by guide RNAs of a CRISPR library suitable for use in humans, e.g., taking into account the expected limitation on guide RNA library size. In some embodiments, the genes selected comprise genes that are expected to influence CAR-T cell cancer killing efficacy or persistence and control genes that are not expected to influence CAR-T cell cancer killing efficacy.

In some aspects, this disclosure describes, a composition comprising a plurality of guide RNA (gRNA) polynucleotides, wherein at least 2 gRNA polynucleotides each comprise a homology region that is complementary to a sense strand or an antisense strand of a different gene encoded by a sequence of any one of SEQ ID NOs: 1-135 or a variant thereof.

In some embodiments, the composition comprises a plurality of guide RNA (gRNA) polynucleotides, wherein at least 10 gRNA polynucleotides each comprise a homology region that is complementary to a sense strand or an antisense strand of a different gene encoded by a sequence of any one of SEQ ID NOs: 1-135 or a variant thereof.

In some embodiments, the composition comprises a plurality of guide RNA (gRNA) polynucleotides, wherein at least 20 gRNA polynucleotides each comprise a homology region that is complementary to a sense strand or an antisense strand of a different gene encoded by a sequence of any one of SEQ ID NOs: 1-135 or a variant thereof.

In some embodiments, the composition comprises a plurality of guide RNA (gRNA) polynucleotides, wherein at least 50 gRNA polynucleotides each comprise a homology region that is complementary to a sense strand or an antisense strand of a different gene encoded by a sequence of any one of SEQ ID NOs: 1-135 or a variant thereof.

In some embodiments, the composition comprises a plurality of guide RNA (gRNA) polynucleotides, wherein at least 100 gRNA polynucleotides each comprise a homology region that is complementary to a sense strand or an antisense strand of a different gene encoded by a sequence of any one of SEQ ID NOs: 1-135 or a variant thereof.

In some embodiments, the composition comprises a plurality of guide RNA (gRNA) polynucleotides, wherein at least 135 gRNA polynucleotides each comprise a homology region that is complementary to a sense strand or an antisense strand of a different gene encoded by a sequence of any one of SEQ ID NOs: 1-135 or a variant thereof.

In some aspects, this application discloses a composition comprising a plurality of guide RNA (gRNA) polynucleotides, wherein at least 2 gRNA polynucleotides each comprise a homology region complementary to a different gene sequence of any one of SEQ ID NOs: 1-135 or a variant thereof.

In some embodiments, at least 10 gRNA polynucleotides each comprise a homology region complementary to a different gene sequence of any one of SEQ ID NOs: 1-135 or a variant thereof. In some embodiments, at least 20 gRNA polynucleotides each comprise a homology region complementary to a different gene sequence of any one of SEQ ID NOs: 1-135 or a variant thereof. In some embodiments, at least 50 gRNA polynucleotides each comprise a homology region complementary to a different gene sequence of any one of SEQ ID NOs: 1-135 or a variant thereof. In some embodiments, at least 100 gRNA polynucleotides each comprise a homology region complementary to a different gene sequence of any one of SEQ ID NOs: 1-135 or a variant thereof. In some embodiments, at least 135 gRNA polynucleotides each comprise a homology region complementary to a different gene sequence of any one of SEQ ID NOs: 1-135 or a variant thereof.

In some embodiments, the composition comprises at least 2 gRNA polynucleotides per gene sequence, wherein the at least 2 gRNA polynucleotides per gene sequence comprises different sequences. In some embodiments, the composition comprises 8 gRNA polynucleotides per gene sequence, wherein the 8 gRNA polynucleotides per gene sequence comprises different sequences. In some embodiments, the at least 2 or at least 8 gRNA polynucleotides per gene sequence are complementary to non-overlapping regions of the same gene sequence.

In some embodiments, the composition comprises one or more negative control gRNA polynucleotides. In some embodiments, the negative control gRNA polynucleotides comprise a randomly generated homology region. In some embodiments, the negative control gRNA polynucleotides comprise homology regions that are complementary to genes that are not known to be involved in T cell function. In some embodiments, the negative control gRNA polynucleotides comprise homology regions that are complementary intergenic regions.

In some embodiments, the composition comprises at least one gRNA polynucleotide comprising a sequence of any one of SEQ ID NOs: 136-1315. In some embodiments, the composition comprising at least 10 gRNA polynucleotides each comprising a different sequence of any one of SEQ ID NOs: 136-1315. In some embodiments, the composition comprising at least 100 gRNA polynucleotides each comprising a different sequence of any one of SEQ ID NOs: 136-1315. In some embodiments, the composition comprising at least 1000 gRNA polynucleotides each comprising a different sequence of any one of SEQ ID NOs: 136-1315. In some aspects, this application discloses, a composition comprising a plurality of gRNA polynucleotides comprising the sequences of SEQ ID NOs: 136-1315. In some embodiments, the plurality of guide RNA polynucleotides consists of at most 200,000 gRNA polynucleotides having different sequences. In some embodiments, the plurality of guide RNA polynucleotides consists of at most 100,000 gRNA polynucleotides having different sequences. In some embodiments, the plurality of guide RNA polynucleotides consists of at most 50,000 gRNA polynucleotides having different sequences. In some embodiments, the plurality of guide RNA polynucleotides consists of at most 25,000 gRNA polynucleotides having different sequences.

In some embodiments, the plurality of guide RNA polynucleotides consists of at most 15,000 gRNA polynucleotides having different sequences. In some embodiments, the plurality of guide RNA polynucleotides consists of at most 10,000 gRNA polynucleotides having different sequences. In some embodiments the plurality of guide RNA polynucleotides consists of at most 5,000 gRNA polynucleotides having different sequences. In some embodiments the plurality of guide RNA polynucleotides consists of at most 2,000 gRNA polynucleotides having different sequences. In some embodiments, the plurality of guide RNA polynucleotides consists of at most 1,080 gRNA polynucleotides having different sequences. In some embodiments, the plurality of guide RNA polynucleotides consists of at most 500 gRNA polynucleotides having different sequences. In some embodiments, the plurality of guide RNA polynucleotides consists of at most 250 gRNA polynucleotides having different sequences. In some embodiments, the plurality of guide RNA polynucleotides consists of at most 100 gRNA polynucleotides having different sequences. In some embodiments, the plurality of guide RNA polynucleotides consists of at most 50 gRNA polynucleotides having different sequences. In some embodiments, the plurality of guide RNA polynucleotides consists of at most 10 gRNA polynucleotides having different sequences.

In some embodiments, at least some of the plurality of gRNA polynucleotides are not cross-reactive with more than one gene. In some embodiments, the composition does not comprise a gRNA homology region that is complementary to a sense strand or an antisense strand of a gene encoding a p53, Rb, PTEN, BRCA1, and/or BRCA2, or a variant thereof. In some embodiments, the composition does not comprise a gRNA that is complementary to a sense strand or an antisense strand of a gene encoding P53, PTEN, APC, P16INK4a, P15INK4b, Cadherin-1, RB1, BRCA1, Wilms tumor 1, STK11, Smad4, BRCA2, CHEK2, P14arf, P21, P73, PTCH1, and/or MSH2, or a variant thereof.

In some embodiments, at least 1 gRNA polynucleotide comprises a homology region that is complementary to a sense strand or an antisense strand of a gene encoded by IL2RA (SEQ ID NO: 65) or a variant thereof.

In some embodiments, at least 1 gRNA polynucleotide comprises a homology region that is complementary to a sense strand or an antisense strand of a gene encoded by GATA3 (SEQ ID NO: 46) or a variant thereof.

In some embodiments, at least 1 gRNA polynucleotide comprises a homology region that is complementary to a sense strand or an antisense strand of a gene encoded by AGPS (SEQ ID NO: 3) or a variant thereof.

In some embodiments, at least 1 gRNA polynucleotide comprises a homology region that is complementary to a sense strand or an antisense strand of a gene encoded by PTPN2 (SEQ ID NO: 98) or a variant thereof.

In some embodiments, at least 1 gRNA polynucleotide comprises a homology region that is complementary to a sense strand or an antisense strand of a gene encoded by LAG3 (SEQ ID NO: 76) or a variant thereof.

In some embodiments, at least 1 gRNA polynucleotide comprises a homology region that is complementary to a sense strand or an antisense strand of a gene encoded by PDCD1 (SEQ ID NO: 91) or a variant thereof.

In some embodiments, at least 1 gRNA polynucleotide comprises a homology region that is complementary to a sense strand or an antisense strand of a gene encoded by TGFBR2 (SEQ ID NO: 121) or a variant thereof.

In some embodiments, at least 1 gRNA polynucleotide comprises a homology region that is complementary to a sense strand or an antisense strand of a gene encoded by RARA (SEQ ID NO: 99) or a variant thereof.

In some embodiments, at least 1 gRNA polynucleotide comprises a homology region that is complementary to a sense strand or an antisense strand of a gene encoded by SmarcB1 (SEQ ID NO: 113) or a variant thereof.

In some embodiments, at least 1 gRNA polynucleotide comprises a homology region that is complementary to a sense strand or an antisense strand of a gene encoded by CDKN1B (SEQ ID NO: 18) or a variant thereof.

In some embodiments, at least 1 gRNA polynucleotide comprises a homology region that is complementary to a sense strand or an antisense strand of a gene encoded by RunX (SEQ ID NO: 107) or a variant thereof.

In some embodiments, at least 1 gRNA polynucleotide comprises a homology region that is complementary to a sense strand or an antisense strand of a gene encoded by TCF7 (SEQ ID NO: 118) or a variant thereof.

In some embodiments, at least 2 gRNA polynucleotides each comprise a homology region that is complementary to a sense strand or an antisense strand of a different gene encoded by any one of IL2RA (SEQ ID NO: 65) or a variant thereof, GATA3 (SEQ ID NO: 46) or a variant thereof, AGPS (SEQ ID NO: 3) or a variant thereof, PTPN2 (SEQ ID NO: 98) or a variant thereof, LAG3 (SEQ ID NO: 76) or a variant thereof, PDCD1 (SEQ ID NO: 91) or a variant thereof, TGFBR2 (SEQ ID NO: 121) or a variant thereof, RARA (SEQ ID NO: 99) or a variant thereof, SmarcB1 (SEQ ID NO: 113) or a variant thereof, CDKN1B (SEQ ID NO: 18) or a variant thereof, RunX (SEQ ID NO: 107) or a variant thereof, and TCF7 (SEQ ID NO: 118) or a variant thereof.

In some embodiments, at least 5 gRNA polynucleotides each comprise a homology region that is complementary to a sense strand or an antisense strand of a different gene encoded by any one of IL2RA (SEQ ID NO: 65) or a variant thereof, GATA3 (SEQ ID NO: 46) or a variant thereof, AGPS (SEQ ID NO: 3) or a variant thereof, PTPN2 (SEQ ID NO: 98) or a variant thereof, LAG3 (SEQ ID NO: 76) or a variant thereof, PDCD1 (SEQ ID NO: 91) or a variant thereof, TGFBR2 (SEQ ID NO: 121) or a variant thereof, RARA (SEQ ID NO: 99) or a variant thereof, SmarcB1 (SEQ ID NO: 113) or a variant thereof, CDKN1B (SEQ ID NO: 18) or a variant thereof, RunX (SEQ ID NO: 107) or a variant thereof, and TCF7 (SEQ ID NO: 118) or a variant thereof.

In some embodiments, at least 7 gRNA polynucleotides each comprise a homology region that is complementary to a sense strand or an antisense strand of a different gene encoded by any one of IL2RA (SEQ ID NO: 65) or a variant thereof, GATA3 (SEQ ID NO: 46) or a variant thereof, AGPS (SEQ ID NO: 3) or a variant thereof, PTPN2 (SEQ ID NO: 98) or a variant thereof, LAG3 (SEQ ID NO: 76) or a variant thereof, PDCD1 (SEQ ID NO: 91) or a variant thereof, TGFBR2 (SEQ ID NO: 121) or a variant thereof, RARA (SEQ ID NO: 99) or a variant thereof, SmarcB1 (SEQ ID NO: 113) or a variant thereof, CDKN1B (SEQ ID NO: 18) or a variant thereof, RunX (SEQ ID NO: 107) or a variant thereof, and TCF7 (SEQ ID NO: 118) or a variant thereof.

In some embodiments, 10 gRNA polynucleotides each polynucleotides each comprise a homology region that is complementary to a sense strand or an antisense strand of a different gene encoded by any one of IL2RA (SEQ ID NO: 65) or a variant thereof, GATA3 (SEQ ID NO: 46) or a variant thereof, AGPS (SEQ ID NO: 3) or a variant thereof, PTPN2 (SEQ ID NO: 98) or a variant thereof, LAG3 (SEQ ID NO: 76) or a variant thereof, PDCD1 (SEQ ID NO: 91) or a variant thereof, TGFBR2 (SEQ ID NO: 121) or a variant thereof, RARA (SEQ ID NO: 99) or a variant thereof, SmarcB1 (SEQ ID NO: 113) or a variant thereof, CDKN1B (SEQ ID NO: 18) or a variant thereof, RunX (SEQ ID NO: 107) or a variant thereof, and TCF7 (SEQ ID NO: 118) or a variant thereof.

In some embodiments, 14 gRNA polynucleotides each polynucleotides each comprise a homology region that is complementary to a sense strand or an antisense strand of a different gene encoded by any one of IL2RA (SEQ ID NO: 65) or a variant thereof, GATA3 (SEQ ID NO: 46) or a variant thereof, AGPS (SEQ ID NO: 3) or a variant thereof, PTPN2 (SEQ ID NO: 98) or a variant thereof, LAG3 (SEQ ID NO: 76) or a variant thereof, PDCD1 (SEQ ID NO: 91) or a variant thereof, TGFBR2 (SEQ ID NO: 121) or a variant thereof, RARA (SEQ ID NO: 99) or a variant thereof, SmarcB1 (SEQ ID NO: 113) or a variant thereof, CDKN1B (SEQ ID NO: 18) or a variant thereof, RunX (SEQ ID NO: 107) or a variant thereof, and TCF7 (SEQ ID NO: 118) or a variant thereof.

In some embodiments, the composition comprises: a first gRNA polynucleotide comprising a homology region that is complementary to a sense strand or an antisense strand of a gene encoded by IL2RA (SEQ ID NO: 65) or a variant thereof; a second gRNA polynucleotide comprising a homology region that is complementary to a sense strand or an antisense strand of a gene encoded by GATA3 (SEQ ID NO: 46) or a variant thereof; a third gRNA polynucleotide comprising a homology region that is complementary to a sense strand or an antisense strand of a gene encoded by AGPS (SEQ ID NO: 3) or a variant thereof; a fourth gRNA polynucleotide comprising a homology region that is complementary to a sense strand or an antisense strand of a gene encoded by PTPN2 (SEQ ID NO: 98) or a variant thereof; a fifth gRNA polynucleotide comprising a homology region that is complementary to a sense strand or an antisense strand of a gene encoded by LAG3 (SEQ ID NO: 76) or a variant thereof; a sixth gRNA polynucleotide comprising a homology region that is complementary to a sense strand or an antisense strand of a gene encoded by PDCD1 (SEQ ID NO: 91) or a variant thereof; a seventh gRNA polynucleotide comprising a homology region that is complementary to a sense strand or an antisense strand of a gene encoded by TGFBR2 (SEQ ID NO: 121) or a variant thereof; a eighth gRNA polynucleotide comprising a homology region that is complementary to a sense strand or an antisense strand of a gene encoded by RARA (SEQ ID NO: 99) or a variant thereof; a nineth gRNA polynucleotide comprising a homology region that is complementary to a sense strand or an antisense strand of a gene encoded by SmarcB1 (SEQ ID NO: 113) or a variant thereof; a tenth gRNA polynucleotide comprising a homology region that is complementary to a sense strand or an antisense strand of a gene encoded by CDKN1B (SEQ ID NO: 18) or a variant thereof or a variant thereof; a eleventh gRNA polynucleotide comprising a homology region that is complementary to a sense strand or an antisense strand of a gene encoded by RunX (SEQ ID NO: 107) or a variant thereof; and/or a twelve gRNA polynucleotide comprising a homology region that is complementary to a sense strand or an antisense strand of a gene encoded by TCF7 (SEQ ID NO: 118) or a variant thereof;

In some embodiments, the composition does not comprise a gRNA polynucleotide comprising a homology region that is complementary to a sense strand or an antisense strand of a gene encoded by RASA2 (SEQ ID NO: 102).

In some embodiments, the composition does not comprise a gRNA polynucleotide comprising a homology region that is complementary to a sense strand or an antisense strand of a gene encoded by one or more of RASA2 (SEQ ID NO: 102) or a variant thereof, TBL1XR1 (SEQ ID NO: 117) or a variant thereof, MEF2D (SEQ ID NO: 80) or a variant thereof, ARIH2 (SEQ ID NO: 6) or a variant thereof, NDUFB10 (SEQ ID NO: 82) or a variant thereof, STAT3 (SEQ ID NO: 115) or a variant thereof, ELOB (SEQ ID NO: 33) or a variant thereof, IRF2 (SEQ ID NO: 67) or a variant thereof, SMARCA4 (SEQ ID NO: 112) or a variant thereof, CYBER4 (SEQ ID NO: 24) or a variant thereof, and LEF1 (SEQ ID NO: 78) or a variant thereof.

In some embodiments, the composition does not comprise a gRNA polynucleotide comprising a homology region that is complementary to a sense strand or an antisense strand of a gene encoded by at least 3 of RASA2 (SEQ ID NO: 102) or a variant thereof, TBL1XR1 (SEQ ID NO: 117) or a variant thereof, MEF2D (SEQ ID NO: 80) or a variant thereof, ARIH2 (SEQ ID NO: 6) or a variant thereof, NDUFB10 (SEQ ID NO: 82) or a variant thereof, STAT3 (SEQ ID NO: 115) or a variant thereof, ELOB (SEQ ID NO: 33) or a variant thereof, IRF2 (SEQ ID NO: 67) or a variant thereof, SMARCA4 (SEQ ID NO: 112) or a variant thereof, CYB5R4 (SEQ ID NO: 24) or a variant thereof, and LEF1 (SEQ ID NO: 78) or a variant thereof.

In some embodiments, the composition does not comprise a gRNA polynucleotide comprising a homology region that is complementary to a sense strand or an antisense strand of a gene encoded by at least 5 of RASA2 (SEQ ID NO: 102) or a variant thereof, TBL1XR1 (SEQ ID NO: 117) or a variant thereof, MEF2D (SEQ ID NO: 80) or a variant thereof, ARIH2 (SEQ ID NO: 6) or a variant thereof, NDUFB10 (SEQ ID NO: 82) or a variant thereof, STAT3 (SEQ ID NO: 115) or a variant thereof, ELOB (SEQ ID NO: 33) or a variant thereof, IRF2 (SEQ ID NO: 67) or a variant thereof, SMARCA4 (SEQ ID NO: 112) or a variant thereof, CYB5R4 (SEQ ID NO: 24) or a variant thereof, and LEF1 (SEQ ID NO: 78) or a variant thereof.

In some embodiments, the plurality of gRNA polynucleotides are Cas9 protein gRNA polynucleotides or a Cas12 protein gRNA polynucleotides. In some embodiments, the plurality of gRNA polynucleotides comprises sequences complementary to at most 15,000 different gene sequences. In some embodiments, the plurality of gRNA polynucleotides comprise sequences complementary to at most 10,000 different gene sequences. In some embodiments, the plurality of gRNA polynucleotides comprises sequences complementary to at most 5,000 different gene sequences. In some embodiments, the plurality of gRNA polynucleotides comprises sequences complementary to at most 2,500 different gene sequences. In some embodiments, the plurality of gRNA polynucleotides comprises sequences complementary to at most 1,000 different gene sequences. In some embodiments, the plurality of gRNA polynucleotides comprise sequences complementary to at most 500 different gene sequences. In some embodiments, the plurality of gRNA polynucleotides comprises sequences complementary to at most 200 different gene sequences. In some embodiments, the plurality of gRNA polynucleotides comprises sequences complementary to at most 135 different gene sequences. In some embodiments, the composition is for use in a human CAR-T cell screen.

In some aspects, this application discloses a guide RNA polynucleotide comprising a sequence of any one of SEQ ID NOs: 136-1315.

In some aspects, this application discloses a plasmid comprising any one of the gRNA polynucleotides of any one of the compositions described herein or the guide RNAs described herein.

In some aspects, this application discloses s plasmid library, comprising at least 2 plasmids any one of the gRNA polynucleotides of any one of the compositions described herein or the guide RNAs described herein. In some embodiments, at least some of the at least 2 plasmids comprise a gRNA polynucleotide of the composition of any one of claims 1-72 or a guide RNA polynucleotide of claim 74. In some embodiments, each plasmid comprises a gRNA polynucleotide of the composition described herein or a guide RNA polynucleotide described herein.

In some aspects, this application discloses a plasmid library comprising each of SEQ ID NOs: 136-1315 encoded on a different plasmid.

In some aspects, this application discloses a gRNA vector comprising: (1) a first gRNA polynucleotide comprising a homology region that is complementary to a sense strand or an antisense strand encoded by a the TCR/CD3 complex (SEQ ID NOs: 1383-1398) of a T cell, and (2) a second gRNA polynucleotide.

In some embodiments, the second gRNA polynucleotide comprises any one of the gRNA polynucleotides of the composition described herein or a guide RNA described herein. In some embodiments, the first gRNA polynucleotide and the second gRNA polynucleotide are in an inverted orientation. In some embodiments, the first gRNA polynucleotide and the second gRNA polynucleotide are in a tandem orientation. In some embodiments, each gRNA encoding sequence is operably linked to a promoter. In some embodiments, the promoter is a constitutively active promoter or an inducible promoter. In some embodiments, the promoter is selected from the group consisting of U6 and EF1a promoters. In some embodiments, the promoter is a U6 promotor, or an H1 promoter, optionally wherein the first gRNA polynucleotide is operably linked to a U6 promoter and the second gRNA polynucleotide is operably linked to a H1 promoter.

In some embodiments, the gRNA vector further comprises a selectable marker, optionally wherein the selectable marker is compatible for use in humans. In some embodiments, the selectable marker is selected from the group consisting of LGNFR, EGFR, CD19, CD20, CD34 and truncated versions thereof. In some embodiments, the selectable marker is operably linked to a promoter, optionally a weak promoter. In some embodiments, the promoter is selected from the group consisting of PGK, CMV, EF1a, and tissue-specific promoters.

In some embodiments, the vector is an adeno-associated vector, a retro-viral vector, or a lentiviral vector. In some embodiments, the lentiviral vector is a third-generation self-inactivating (SIN) lentiviral vector.

In some embodiments, the vector comprises a suicide gene. In some embodiments, the suicide gene is selected from the group consisting of icaspase9, tEGFR, tCD29, CD20, and tHer2. In some embodiments, the gRNA vector comprises a nucleic acid sequence of any one of SEQ ID NOs: 1318.

In some embodiments, the gRNA vector further comprises a polynucleotide encoding a CRISPR protein. In some embodiments, the CRISPR protein is Cas9 or Cas12.

In some aspects, this application discloses a gRNA vector library, comprising at least 2 gRNA vectors described herein. In some embodiments, each vector comprises a gRNA polynucleotide of a composition described herein or a guide RNA described herein. In some embodiments, at least 10 vectors of the library each comprise a different gRNA polynucleotide homology region that is complementary to a sense strand or an antisense strand of a different gene encoded by any one of SEQ ID NOs: 1-135 or a variant thereof.

In some embodiments, the gRNA vector library is a current good manufacturing practice (cGMP)-grade gRNA vector library. In some embodiments, the cGMP-grade gRNA vector library is a cGMP-grade gRNA lentiviral gRNA vector library.

In some embodiments, at least 20 vectors of the library each comprise a different gRNA polynucleotide homology region that is complementary to a sense strand or an antisense strand of a different gene encoded by any one of SEQ ID NOs: 1-135 or a variant thereof. In some embodiments, at least 50 vectors of the library each comprise a different gRNA polynucleotide homology region that is complementary to a sense strand or an antisense strand of a different gene encoded by any one of SEQ ID NOs: 1-135 or a variant thereof. In some embodiments, at least one-hundred vectors of the library each comprise a different gRNA polynucleotide homology region that is complementary to a sense strand or an antisense strand of a different gene encoded by any one of SEQ ID NOs: 1-135 or a variant thereof. In some embodiments, at least one-hundred thirty-five vectors of the library each comprise a different gRNA polynucleotide homology region that is complementary to a sense strand or an antisense strand of a different gene encoded by any one of SEQ ID NOs: 1-135 or a variant thereof.

In some aspects, this application discloses a CAR vector comprising a polynucleotide that encodes a chimeric antigen receptor (CAR). In some embodiments, the vector is a self-inactivating vector. In some embodiments, the vector is an adeno-associated vector, a retro-viral vector, or a lentiviral vector. In some embodiments, the vector is a third-generation self-inactivating (SIN) lentiviral vector.

In some embodiments, the CAR is operably linked to a promoter, optionally an EF1a or EF1a-short promoter.

In some embodiments, the vector comprises a suicide gene. In some embodiments, the suicide gene is selected from the group consisting of icaspase9, tEGFR, tCD29, CD20, and tHer2. In some embodiments, the vector comprises a reporter. In some embodiments, the reporter is selected from the group consisting of truncated CD34, tEGFR, tCD19, tCD20, tCD34, and tHer2. In some embodiments, the CAR comprises a polynucleotide encoding an antigen binding domain that binds to any one of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain.

In some embodiments, the CAR comprises a polynucleotide encoding an antigen binding domain that binds to mesothelin. In some embodiments, the CAR further comprises a transmembrane domain and an intracellular signaling domain. In some embodiments, the transmembrane domain is selected from the group consisting of alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the intracellular signaling domain is selected from the group consisting of CD28, 4-1BB, CD27, TCR-zeta, FcR-gamma, FcR-beta, CD3-gamma, CD3-theta, CD3-sigma, CD3-eta, CD3-epsilon, CD3-zeta, CD22, CD79a, CD79b, and CD66d.

In some embodiments, a 2A ribosomal skip element or an internal ribosome entry site (IRES) is downstream of the CAR. In some embodiments, the CAR vector comprises a sequence of SEQ ID NO: 1319. In some aspects, this application discloses a composition comprising the gRNA vector of any one of claims as described herein and a CAR vector of as described herein.

In some aspects, this application discloses, a CAR-T cell comprising the composition comprising the gRNA vector of any one of claims as described herein and a CAR vector of as described herein. In some aspects, this application discloses a CAR-T cell comprising one, two, or three of: a mutation in a gene corresponding to the gene sequence of any one of SEQ ID NOs: 1-135 or a variant thereof, a gRNA polynucleotide of the composition as described herein, a plasmid as described herein, or a gRNA vector as described herein. In some embodiments, the CAR-T cell further comprises a CAR vector as described herein.

In some embodiments, the mutation is a loss of function mutation. In some embodiments, the mutation is an insertion or a deletion. In some embodiments, the insertion or deletion mutation causes a frameshift. In some embodiments, the CAR-T cell further comprises an mRNA encoding a CRISPR protein. In some embodiments, the CAR-T cell is CD34 positive and CD3 negative. In some embodiments, a T cell used to make the CAR-T cell is from a mammalian subject. In some embodiments, a T cell used to make the CAR-T cell is from a human subject. In some embodiments, a T cell used to produce the CAR-T cell is collected using leukapheresis.

In some embodiments, the CAR-T cell is activated. In some embodiments, the CAR-T cell is activated using anti-CD3 and anti-CD28 antibodies. In some embodiments, the CAR-T cell is activated using anti-CD3 antibody. In some embodiments, the CAR-T cell is activated using anti-CD3 antibody and IL-2. In some embodiments, the CAR-T cell is activated using anti-CD3 antibody, IL-2 and PHA. In some embodiments, the anti-CD3 antibody, the anti-CD28 antibody, the IL2 and the PHA are either soluble or plate-bound. In some embodiments, the CRISPR protein is Cas9 or Cas12. In some embodiments, the cell does not comprise a DNA polynucleotide encoding a CRISPR protein. In some embodiments, the CAR-T cell is CD34 positive, CD3 negative and LNGFR positive.

In some embodiments, the CAR-T cells of the library are selected from the CAR-T cells described herein. In some embodiments, at least 2 CAR-T Cells of the library each comprise a different gRNA polynucleotide comprising a homology region that is complementary to a different gene sequence of any one of SEQ ID NOs: 1-135 or a variant thereof. In some embodiments, at least 10 CAR-T Cells of the library each comprise a different gRNA polynucleotide comprising a homology region that is complementary to a different gene sequence of any one of SEQ ID NOs: 1-135 or a variant thereof. In some embodiments, at least 20 CAR-T Cells of the library each comprise a different gRNA polynucleotide comprising a homology region that is complementary to a different gene sequence of any one of SEQ ID NOs: 1-135 or a variant thereof. In some embodiments, at least 50 CAR-T Cells of the library each comprise a different gRNA polynucleotide comprising a homology region that is complementary to a different gene sequence of any one of SEQ ID NOs: 1-135 or a variant thereof. In some embodiments, at least 100 CAR-T Cells of the library each comprise a different gRNA polynucleotide comprising a homology region that is complementary to a different gene sequence of any one of SEQ ID NOs: 1-135 or a variant thereof. In some embodiments, 135 CAR-T Cells of the library each comprise a different gRNA polynucleotide comprising a homology region that is complementary to a different gene sequence of any one of SEQ ID NOs: 1-135 or a variant thereof.

In some embodiments, SEQ ID NOs: 136-1315 are each present in at least one CAR-T cell.

In some aspects, this application discloses a method of producing a mutant CAR-T cell library comprising: (a) activating T cells, (b) transfecting the T cells with the gRNA vector library described herein, (c) transfecting the T cells with the CAR vector described herein, and (d) introducing a CRISPR protein encoding mRNA or a CRISPR protein into the T cell.

In some aspects, this application discloses a method of producing a mutant CAR-T cell library comprising: (a) activating T cells, (b) transfecting the T cells with the gRNA vector library described herein, and (c) transfecting the T cells with the CAR vector of any one of claims C1-C11, wherein the gRNA vector or the CAR vector comprises a polynucleotide encoding a CRISPR protein.

In some embodiments, the method further comprises collecting T cells from a subject. In some embodiments, the method further comprises purifying T cells that comprise a CAR vector and comprise a CRISPR induced gene mutation. In some embodiments, the T cells are collected using leukapheresis. In some embodiments, the T cells are collected from a human or mouse subject. In some embodiments, the T cells are activated using anti-CD3 and anti-CD28 antibodies. In some embodiments, the gRNA vector library is transduced with a multiplicity of infection (MOI) between 0.1-1. In some embodiments, the MOI is 0.3-0.7. In some embodiments, the MOI is 0.4-0.6. In some embodiments, the MOI is 0.5. In some embodiments, the CAR vector is transduced with an MOI up to 20.

In some embodiments, the CRISPR protein is Cas9 or Cas12. In some embodiments, the CRISPR protein mRNA or CRISPR protein is introduced using electroporation. In some embodiments, the CRISPR mRNA is expressed from the gRNA vector or the CAR vector. In some embodiments, electroporation is performed 3-7 days after transduction with the gRNA vector and the CAR vector. In some embodiments, the method further comprises selecting T cells that are CD3 negative, CD34 positive, and optionally LNGFR positive. In some embodiments, selection is performed using magnetic beads and/or flow cytometry sorting. In some aspects, this application discloses a mutant CAR-T cell library produced using the methods described herein.

In some aspects, this application discloses a method of identifying gRNA polynucleotides associated with CAR-T cell efficacy in vivo comprising: (a) administering to a subject a mutant CAR-T cell library of any one of claims E1-E7 or F15, (b) collecting one or more samples comprising a plurality of mutant CAR-T cells of the mutant CAR-T cell library from the subject, (c) sequencing the gRNA polynucleotides from the mutant CAR-T cells collected in (b), and
(d) evaluating the change in relative abundance of each gRNA polynucleotide based on the sequencing in (c).

In some embodiments, the method further comprises: (e) identifying a gene associated with a gRNA polynucleotide having an increase in relative abundance; (f) producing a modified CAR-T cell comprising a mutation in the gene identified in (e); and (g) administering the modified CAR-T cell to the subject.

In some embodiments, the mutant CAR-T cell library comprises a genome-wide CAR-T cell mutant library. In some embodiments, the library comprises negative control CAR-T cells. In some embodiments, the negative control CAR-T cells comprise a negative control gRNA polynucleotide described herein. In some embodiments, the subject is human. In some embodiments, the subject has cancer. In some embodiments, the cancer is selected from the group consisting of ovarian cancer, pancreatic cancer, lung cancer, prostate cancer, breast cancer, AML, multiple myeloma, and B cell lymphomas. In some embodiments, the CAR comprises an antigen binding domain that binds to an antigen expressed by the cancer.

In some embodiments, the CAR comprises an antigen binding domain selected from the group consisting of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL. In some embodiments, the samples are collected from the subject's blood. In some embodiments, at least 3 samples are collected from the subject. In some embodiments, at least 5 samples are collected via venipuncture, apheresis, or resection of tumor. In some embodiments, samples are collected from the subject after step (a) at one or more of day 3, day 7, day 10, day 14, day 21, month 1, month 2, month 3, month 6, month 9, month 12, month 15, month 18, month 21, and month 24 after administering the mutant CAR-T cell library. In some embodiments, a sample is collected from the subject after step (a) at day 21 after administering the mutant CAR-T cell library.

In some embodiments, the method further comprises extracting the mutant CAR T cell from the sample prior to sequencing. In some embodiments, extraction is performed by isolating T cells that are CD3 negative, CD34 positive, and optionally LNGFR positive.

In some embodiments, the method further comprises isolating genomic DNA from the mutant CAR-T cells. In some embodiments, the method further comprises isolating gRNA polynucleotides from the mutant CAR-T cells. In some embodiments, the method further comprises isolating the RNA from the mutant CAR-T cells and optionally sequencing the RNA. In some embodiments, the method further comprises amplifying the gRNA polynucleotides of the mutant CAR-T cells to produce amplicons of any thereof. In some embodiments, the gRNA polynucleotides amplified are genomic DNA, vector DNA, or RNA. In some embodiments, primers of SEQ ID NOs: 1324-1325 are used for amplification. In some embodiments, the gRNA polynucleotides or amplicons thereof are sequenced. In some embodiments, the gRNA polynucleotide or amplicons thereof are sequenced using next-generation sequencing.

In some embodiments, the efficacy of each mutant CAR-T cell is evaluated based on persistence in vivo and tumor cytotoxicity. In some embodiments, persistence in vivo and tumor cytotoxicity are measured using flow cytometry and PCR. In some embodiments, quantifying persistence is performed by calculating the relative change in each gRNA over time.

In some embodiments, quantifying persistence is performed by calculating the relative change in each gRNA over time compared to a gRNA polynucleotide from a negative control CAR-T cell. In some embodiments, quantifying persistence is performed by calculating the relative change in each gRNA over time across at least 3 time points and calculating a slope of relative change. In some embodiments, tumor cytotoxicity is quantified using clinical imaging and/or bone marrow evaluation. In some aspects, this application discloses a CAR natural killer (NK) cell comprising the composition described herein.

In some aspects, this application discloses a CAR-NK cell comprising one, two, or three of: a mutation in a gene corresponding to the gene sequence of any one of SEQ ID NOS: 1-135 or a variant thereof, a gRNA polynucleotide of the composition described herein, a plasmid described herein, or a gRNA vector described herein. In some embodiments, the CAR-NK cell further comprises a CAR vector. In some embodiments, the mutation is a loss of function mutation. In some embodiments, the mutation is an insertion or a deletion. In some embodiments, the insertion or deletion mutation causes a frameshift.

In some embodiments, the CAR-NK further comprises an mRNA encoding a CRISPR protein and/or a CRISPR protein. In some embodiments, the NK cell is CD34 positive and CD3 negative. In some embodiments, a NK cell used to make the NK cell is from a mammalian subject. In some embodiments, a NK cell used to make the NK cell is from a human subject. In some embodiments, a NK cell used to produce the NK cell is collected from blood, optionally cord blood or peripheral blood. In some embodiments, the NK cell is activated. In some embodiments, the NK cell is activated using irradiated feeder cells, IL-2 and IL-15. In some embodiments, the NK cell is activated using irradiated K562 cells. In some embodiments, the NK cell is activated using IL-21. In some embodiments, the NK cell is activated using 4-1BBL, IL-2, and irradiated K562 cells expressing membrane bound IL-15. In some embodiments, the CRISPR protein is Cas9 or Cas12. In some embodiments, the NK cell does not comprise a DNA polynucleotide encoding a CRISPR protein. In some embodiments, the NK cell is CD34 positive, CD3 negative and LNGFR positive.

In some embodiments, the NK cells of the library are selected from NK cells described herein. In some embodiments, at least 2 CAR-NK Cells of the library each comprise a different gRNA polynucleotide comprising a homology region that is complementary to a different gene sequence of any one of SEQ ID NOs: 1-135 or a variant thereof. In some embodiments, at least 10 CAR-NK Cells of the library each comprise a different gRNA polynucleotide comprising a homology region that is complementary to a different gene sequence of any one of SEQ ID NOs: 1-135 or a variant thereof. In some embodiments, at least 20 CAR-NK Cells of the library each comprise a different gRNA polynucleotide comprising a homology region that is complementary to a different gene sequence of any one of SEQ ID NOs: 1-135 or a variant thereof. In some embodiments, at least 50 CAR-NK Cells of the library each comprise a different gRNA polynucleotide comprising a homology region that is complementary to a different gene sequence of any one of SEQ ID NOs: 1-135 or a variant thereof. In some embodiments, at least 100 CAR-NK Cells of the library each comprise a different gRNA polynucleotide comprising a homology region that is complementary to a different gene sequence of any one of SEQ ID NOs: 1-135 or a variant thereof. In some embodiments, 135 CAR-NK Cells of the library each comprise a different gRNA polynucleotide comprising a homology region that is complementary to a different gene sequence of any one of SEQ ID NOs: 1-135 or a variant thereof. In some embodiments, SEQ ID NOs: 136-1315 are each present in at least one NK cell.

In some aspects, this application discloses a method of producing a mutant CAR-NK cell library comprising: (a) activating CAR-NK cells, (b) transducing the CAR-NK cells with the gRNA vector library described herein, (c) transducing the CAR-NK cells with the CAR vector described herein, and (d) introducing a CRISPR protein encoding mRNA or a CRISPR protein into the NK cell.

In some aspects, this application discloses a method of producing a mutant CAR-NK cell library comprising: (a) activating CAR-NK cells, (b) transducing the CAR-NK cells with the gRNA vector library described herein, and (c) transducing the CAR-NK cells with the CAR vector described herein, wherein the gRNA vector or the CAR vector comprises a polynucleotide encoding a CRISPR protein.

In some embodiments, the method of claims further comprises collecting CAR-NK cells from a subject. In some embodiments, the method further comprises purifying CAR-NK cells that comprise a CAR vector and comprise a CRISPR induced gene mutation. In some embodiments, the CAR-NK cells are collected using leukapheresis. In some embodiments, the CAR-NK cells are collected from a human or mouse subject. In some embodiments, the CAR-NK cells are activated using anti-CD3 and anti-CD28 antibodies. In some embodiments, the gRNA vector library is transduced with an multiplicity of infection (MOI) between 0.1-1. In some embodiments, the MOI is 0.3-0.7. In some embodiments, the MOI is 0.4-0.6. In some embodiments, the MOI is 0.5. In some embodiments, the CAR vector is transduced with an MOI up to 20. In some embodiments, the CRISPR protein is Cas9 or Cas12.

In some embodiments, the CRISPR protein mRNA or CRISPR protein is introduced using electroporation. In some embodiments, the CRISPR mRNA is expressed from the gRNA vector or the CAR vector. In some embodiments, electroporation is performed 3-7 days after transduction with the gRNA vector and the CAR vector.

In some embodiments, the method further comprises selecting NK cells that are CD3 negative, CD34 positive. In some embodiments, the method further comprises selecting NK cells that are CD3 negative, CD34 positive and LNGFR positive.

In some embodiments, selection is performed using magnetic beads and/or flow cytometry sorting. In some aspects, this application discloses a mutant CAR-NK cell library produced using the methods described herein.

In some aspects, this application discloses, a method of identifying mutations that increase CAR-NK cell efficacy in vivo comprising: (a) administering to a subject a mutant CAR-NK cell library described herein, (b) collecting one or more samples comprising a plurality of mutant CAR-NK cells of the mutant NK cell library from the subject, (c) sequencing the gRNA polynucleotides from the mutant CAR-NK cells collected in (b), and (d) evaluating the change in relative abundance of each gRNA polynucleotide based on the sequencing in (c).

In some embodiments, the method further comprises (e) identifying a gene associated with a gRNA polynucleotide having an increase in relative abundance; (f) producing a modified CAR-T cell comprising a mutation in the gene identified in (e); and (g) administering the modified CAR-T cell to the subject.

In some embodiments, the mutant CAR-NK cell library comprises a genome-wide NK cell mutant library. In some embodiments, the library comprises negative control CAR-NK cells. In some embodiments, the negative control CAR-NK cells comprise a negative control gRNA polynucleotide a described herein. In some embodiments, the subject is human.

In some embodiments, the subject has cancer. In some embodiments, the cancer is selected from the group consisting of ovarian cancer, pancreatic cancer, lung cancer, prostate cancer, breast cancer, AML, multiple myeloma, and B cell lymphomas. In some embodiments, the CAR comprises an antigen binding domain that binds to an antigen expressed by the cancer.

In some embodiments, the CAR comprises an antigen binding domain selected from the group consisting of CD19, CD79b, TACI, BCMA, MUC1, MUC16, B7H3, mesothelin, CD70, PSMA, PSCA, EGFRVIII, claudin6, binds to any pair of CD19/CD79b, BCMA/TACI, or is a TriPRIL. In some embodiments, the samples are collected from the subject's blood. In some embodiments, at least 3 samples are collected from the subject. In some embodiments, at least 5 samples are collected via venipuncture, apheresis, or resection of tumor. In some embodiments, samples are collected from the subject after step (a) at one or more of day 3, day 7, day 10, day 14, day 21, month 1, month 2, month 3, month 6, month 9, month 12, month 15, month 18, month 21, and month 24 after administering the mutant CAR-T cell library. In some embodiments, a sample is collected from the subject after step (a) at day 21 after administering the mutant CAR-T cell library.

In some embodiments, the method further comprises extracting the mutant NK cell from the sample prior to sequencing. In some embodiments, extraction is performed by isolating NK cells that are CD3 negative, CD34 positive, and optionally LNGFR positive. In some embodiments, the method further comprises isolating genomic DNA from the mutant NK cells. In some embodiments, the method further comprises isolating gRNA polynucleotides from the mutant NK cells. In some embodiments, the method further comprises isolating the RNA from the mutant NK cells and optionally sequencing the RNA. In some embodiments, the method further comprises amplifying the gRNA polynucleotides of the mutant NK cells to produce amplicons of any thereof. In some embodiments, the gRNA polynucleotides amplified are genomic DNA, vector DNA, or RNA. In some embodiments, primers of SEQ ID NOs: 1324-1325 are used for amplification. In some embodiments, the gRNA polynucleotides or amplicons thereof are sequenced. In some embodiments, the gRNA polynucleotide or amplicons thereof are sequenced using next-generation sequencing. In some embodiments, the efficacy of each mutant NK cell is evaluated based on persistence in vivo and tumor cytotoxicity. In some embodiments, persistence in vivo and tumor cytotoxicity are measured using flow cytometry and PCR. In some embodiments, quantifying persistence is performed by calculating the relative change in each gRNA over time. In some embodiments, quantifying persistence is performed by calculating the relative change in each gRNA over time compared to a gRNA polynucleotide from a negative control NK cell. In some embodiments, quantifying persistence is performed by calculating the relative change in each gRNA over time across at least 3 timepoints and calculating a slope of relative change. In some embodiments, tumor cytotoxicity is quantified using clinical imaging and/or bone marrow evaluation.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows a diagram of identifying mutant CAR-T cells that increase CAR-T cell survival in vivo. Patients undergo leukapheresis and their T cells are transduced with two lentiviral vectors: one to express the chimeric antigen receptor (CAR) and truncated CD34 (CD34t) and the other to express 2 guide RNAs (one a barcoded gRNA from the library and one targeting T cell receptor alpha chain—TRAC) and truncated low-affinity nerve growth factor receptor (tLNGFR). 2) Transduced T cells are electroporated with Cas9 to initiate the genetic mutations, allowing for knockout of the gene targeted by the library gRNA and CD3 targeted by the TRAC gRNA. 3) Successfully mutated T cells are negatively selected based loss of CD3 expression using magnetic-activated cell sorting (MACS). 4) Mutated T cells are then positively selected for CD34t to isolate CAR T cells, creating a library of mutant CAR T cells. 5) This library of CAR T cells is then infused back into the patient. 6) After 7-14 days, tumor and blood samples are collected from the patient. The cells are lysed, and the DNA is collected. 7) The DNA is sequenced to identify barcodes of the gRNA expressed by CAR T cells surviving in the tumor or blood, which allows for identification of genes that when knocked out increased CAR T cell survival in these compartments.

FIGS. 2A-2B show the vector maps for the CAR vector and gRNA vector. FIG. 1A shows a CAR vector comprising a nucleic acid sequence encoding a CD34 truncated (CD34t) selectable marker. FIG. 2B shows a gRNA vector comprising 2 guide RNA cassettes, a suicide gene (iCasp9) operably linked to a PGK promoter, and a low-affinity nerve growth factor receptor truncated (LNGFRt) selectable marker.

FIGS. 3A-3E show knockout of multiple genes in primary T cells using double guide cassette vector (pMGH354-CP1780) FIG. 3A shows an experimental workflow for production of double-knockout human T cells expressing drug-inducible Caspase-9 (iCasp9) safety switch and truncated LNGFR electroporated with Cas9. FIG. 3B shows a schematic of lentiviral vector introduced into T cells containing sgRNAs targeting the T cell receptor alpha (TRAC) and CD5 genes followed by iCasp9 safety switch and truncated LNGFR for positive selection. FIG. 3C shows flow cytometry data demonstrating knockout of the T cell receptor complex (TCR). FIG. 3D shows flow cytometry data demonstrating knockout of the CD5 using two different sgRNAs. FIG. 3E shows flow cytometry data showing surface expression of truncated LNGFR in cells transduced with the lentiviral vector.

FIGS. 4A-4C show guide RNA abundance in pooled library determined via sequencing. FIG. 4A shows a schematic of lentiviral vector introduced into T cells containing two sgRNAs followed by drug-inducible Caspase-9 (iCasp9) safety switch and truncated LNGFR. for positive selection. FIG. 4B shows an experimental workflow for production of double-knockout T cells expressing drug-inducible Caspase-9 (iCasp9) safety switch and truncated LNGFR electroporated with Cas9 followed by treatment with Rimducid to induce iCasp9 activation leading to cell death. FIG. 4C shows flow cytometric evaluation of iCasp9 induced cell death by monitoring the loss of CAR+ cells over time.

FIG. 5 shows a schematic of a CRISPR screen in cancer patients.

FIG. 6 shows the functional distribution of the 135 genes targeted by the sgRNA CRISPR (Mario) library.

FIGS. 7A-7D shows construct designs and validation of a gene knockout in human T cell. FIG. 7A shows lentiviral vector designs to be used for in vivo screens. The pCAR vector contains the CAR. The pGuide vector contains a double guide cassette for delivery of two sgRNAs. FIG. 7B shows NGFR reporter expression from the pGuide vector on human T cells. FIG. 7C shows editing efficiency of an example CD5-targeting sgRNA cloned into sgRNA position 1. FIG. 7D shows the editing efficiency of the TRAC-targeting sgRNA cloned into position 2 of the pMario construct.

FIGS. 8A-8C show generation of the mesothelin CAR T cell with CRISPR (Mario) library for in vivo testing. FIG. 8A shows the construct design to create the CAR-T cells with the CRISPR Mario library. FIG. 8B show a schematic of the mesothelin CAR T cell with CRISPR (Mario) library production process. FIG. 8C shows transduction efficiencies of the two lentiviral vectors before and after selection for CD3-negative cells. The CAR construct is labeled using mCherry while the CRISPR library construct is evaluated using the knockout of CD3.

FIG. 9 shows a schematic of the experimental design for the in vivo CRISPR screen in mice.

FIGS. 10A-10B show results of an in vivo loss of function (LOF) CRISPR screen in APSC1 tumor (pancreatic tumor)-bearing mice. FIG. 10A shows the guide distribution comparison showing the ability to capture the full guide library in the tumor-engrafted CAR T cells and spleen-isolated CAR T cells. FIG. 10B shows a hypergeometric test showing enriched and depleted guides in the captured CAR t cells from the tumor and spleen. In addition to RASA2 being depleted and IL2RA being enriched, novel gene targets were identified such as GATA3 and ETS1 that can be of potential importance to CAR T cell functionality.

FIGS. 11A-11B show guide distribution in large-scale plasmid preparations and transduced human T cells. FIG. 11A shows a histogram plot of guide abundance in the original plasmid primary library stock (blue line) and a large-scale plasmid preparation (orange line) using the PCR products flanking the guide region of the plasmid. FIG. 11B shows guide distribution in human primary T cells transduced with lentiviral vector containing the CRISPR library. Lentiviral vector was prepared using either the original plasmid library stock or the large-scale plasmid preparation before being transduced into human normal donor T cells.

FIG. 12 shows a proposed manufacturing process for Meso-Mario-CART cells.

FIG. 13 shows the timeline for the phase I study of the Meso-Mario-CAR-T cells in human patients.

FIG. 14 shows the sample collection and analysis plan for the phase I study of the Meso-Mario-CAR-T cells in human patients.

FIG. 15 shows a construct schematic of the used BCMA CAR (4-1BB costimulatory domain, CD34t transduction marker) and the double guide cassette (LNGFR marker)

FIG. 16 shows the BCMA CAR-T CRISPR screen experimental design. After 21 days of engraftment with 1E6 MM1.s tumor cells, mice are treated with 2E6 double-transduced BCMA+library construct. On day 7, mice are sacrificed, and bone marrow (spine, femur, tibia) is harvested as part of collecting the BCMA CAR-T CRISPR cells.

FIG. 17 shows measurements of tumor killing activity of a BCMA CAR-T cell and the BCMA CAR-T Cell CRISPR library with normal donor 216 T cells, until day 7. Body luminescence imaging (BLI), both by group (left) and single mice. BCMA CAR and double-transduced BCMA+library construct show antitumor activity, while tumor only and library only are not able to control the tumor.

FIG. 18 shows expression using STARS scoring STARS rank scoring, allowing for quantification of significant enriched (highest IL2RA) and depleted hits (highest IRF4).

FIGS. 19A-19B shows enriched and deleted gene knockout in the BCMA CAR-T CRISPR screen. FIG. 19A shows the STARS scoring rank per mice assessment, enriched. From STARS scoring, identifying enriched gene hit that are inside of the top 10 enriched genes in 6 mice or more. For example, IL2RA is inside the top10 enriched hits in all 15 tested mice. FIG. 19B shows the STARS scoring rank per mice assessment, depleted. From STARS scoring, identifying enriched gene hit that are inside of the top 10 enriched genes in 6 mice or more. Example: IRF4 and RASA are inside the top10 depleted hits in all 15 tested mice

FIGS. 20A-20F show results from a BCMA CRISPR CAR-T Screen and determining the period for selection. FIG. 20A shows the BCMA repeat screen experimental design. Analog to FIG. 16, screen is repeated with two other healthy donor T cells. FIG. 20B shows tumor activity measurements, T cells of normal donors 106 and 99, until day 21 BLI, both by group (left) and single mice. BCMA CAR and double-transduced BCMA+library construct show effective and rapid tumor clearance, while tumor only and library only are not able to control the tumor. FIG. 20C shows expression using STARS scoring, measuring day 0 to day 7. Analog to FIG. 18, displaying STARS rank scoring in the other two normal donor T cells, from day 0 to day 7 (in vivo period). The highest enriched CAR-T cell gene CRISPR targets was IL2RA, and the highest depleted was IRF4-both consistent with results from first ND (see FIG. 18). FIG. 20D shows expression using STARS scoring, measuring day 0 to day 21. Analog to FIG. 18, displaying STARS scoring in the other two normal donor T cells, from day 0 to day 21 (in vivo period). Highest enriched CAR-T cell gene CRISPR target was IL2RA, and the highest depleted was STAT3. FIG. 20E shows expression using STARS scoring, measuring day-11 to day 21. Analog to FIG. 18, displaying STARS scoring in the other two normal donor T cells, from day-11 to day 21 (in vitro & in vivo period). Highest enriched CAR-T cell gene CRISPR target was CDKN1B, and the highest depleted was IL2RA. FIG. 20F shows expression using STARS scoring, measuring day-11 to day 7. Highest enriched gene CRISPR targets were PTPN2 and SOCS1, and the highest depleted was IRF4.

FIGS. 21A-21C show production of a current good manufacturing practice cGMP gRNA plasmid library. FIG. 21A shows a first attempt at making the library and resulting low yields and low quality DNA that is not sufficient to produce a cGMP gRNA plasmid library. FIG. 21B shows that increasing antibiotic amount (by two fold or four fold) used cGMP library preparation increases gRNA plasmid library yields. FIG. 21C shows that increasing antibiotic amount produces a gRNA plasmid library of expected yield and quality as determined by restriction digest.

### DETAILED DESCRIPTION

A general challenge in cell therapy (e.g., CAR-T cell therapy) is identifying CAR-T cell modifications (e.g., gene mutations) that improve cell therapy efficacy. This is challenging for a few different reasons. First, while CAR-T cell efficacy in vitro and in mice can inform in human efficacy, CAR-T cells may have different efficacies in humans than in vitro or in mice. Second, predicting how a given mutation (e.g., a loss of function mutation) will affect CAR-T cell efficacy in humans may be difficult, so many mutations may need to be tested to identify a mutation that improves CAR-T cancer killing efficacy. However, individually testing many different modified CAR-T cells in humans is impractical. Third, different subjects may respond differently to different CAR-T cell modifications. For example, a first modified CAR-T cell may have higher efficacy in a first subject but lower efficacy in a second subject compared to a second modified CAR-T cell. In some aspects, this disclosure describes methods and compositions for addressing these challenges. For example, this disclosure describes methods and compositions for screening a library of modified CAR-T cells in vivo (e.g., in humans) in a single experiment to identify CAR-T cell modifications that increase CAR-T cell efficacy (e.g., persistence in vivo).

### General Definitions

The terms “decrease,” “reduced,” “reduction,” or “inhibit” are all used herein to mean a decrease by a statistically significant amount. In some embodiments, “reduce,” “reduction,” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. “Complete inhibition” is a 100% inhibition as compared to a reference level. Where applicable, a decrease can be preferably down to a level accepted as within the range of normal for an individual without a given disorder. The terms “increased,” “increase,” “enhance,” or “activate” are all used herein to mean an increase by a statically significant amount. In some embodiments, the terms “increased,” “increase,” “enhance,” or “activate” can mean an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

A “disease” is a state of health of an animal, for example, a human, wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. In some embodiments, the disease is a cancer or a tumor.

As used herein, the terms “tumor antigen”, “tumor-associated antigen” and “cancer antigen” are used interchangeably to refer to antigens that are differentially expressed by cancer cells and can thereby be exploited in order to target cancer cells. Cancer antigens are antigens that can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), human epidermal growth factor receptor (HER2), mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively. In some embodiments, the tumor-associated antigen is any one of CD19, CD79b, TACI, BCMA, MUC1, MUC16, B7H3, mesothelin, CD70, PSMA, PSCA, EGFRVIII, claudin6, or any pair of CD19/CD79b, or BCMA/TACI.

As used herein, the term “chimeric” refers to the product of the fusion of portions of at least two or more different polynucleotide molecules. In some embodiments, the term “chimeric” refers to a gene expression element produced through the manipulation of known elements or other polynucleotide molecules.

In some embodiments, “activation” can refer to the state of a T cell that has been sufficiently stimulated to induce detectable cellular proliferation. In some embodiments, activation can refer to induced cytokine production. In other embodiments, activation can refer to detectable effector functions.

At a minimum, an “activated T cell” as used herein is a proliferative T cell.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

A “stimulatory ligand,” as used herein, refers to a ligand that when present on an antigen presenting cell (APC) (e.g., a macrophage, a dendritic cell, a B-cell, an artificial APC, and the like) can specifically bind with a cognate binding partner (referred to herein as a “stimulatory molecule” or “costimulatory molecule”) on a T cell, thereby mediating a primary response by the T cell, including, but not limited to, proliferation, activation, initiation of an immune response, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on a T cell that

specifically binds with a cognate stimulatory ligand present on an antigen presenting cell. “Co-stimulatory ligand,” as the term is used herein, includes a molecule on an APC that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, 4-1BBL, OX40L, CD7, B7-1 (CD80), B7-2 (CD86), PD-L1, PD-L2, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, IL T3, IL T4, HVEM, an agonist or antibody that binds Toll-like receptor and a ligand that specifically binds with B7-H3. A co-stimulatory ligand also can include, but is not limited to, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule, BTLA, a Toll-like receptor, CD27, CD28, 4-1BB, OX40, CD30, CD40, PD-1, ICOS, lymphocyte function associated antigen-1 (LFA-1), CD2, CD7, LIGHT, NKG2C, B7-H3, and CD83.

In some embodiments, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene.

The term “cell” or “engineered cell” and their grammatical equivalents as used herein can refer to a cell of human or non-human animal origin.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ligand-mediated receptor activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; ile into Leu or into Val; Leu into ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a

polypeptide) can be a functional fragment of one of the amino acid sequences described herein. As used herein, a “functional fragment” is a fragment or segment of a peptide that retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

The term “polynucleotide” is used herein interchangeably with “nucleic acid molecule” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. In some embodiments, the nucleic acid molecule is a heterologous nucleic acid molecule. As used herein the term, “heterologous nucleic acid molecule” refers to a nucleic acid molecule that does not naturally exist within a given cell.

A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide may be a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “gene” may refer to a nucleic acid sequence which is transcribed (DNA) to RNA in vitro or in vivo when operably linked to appropriate regulatory sequences. The gene may or may not include regions preceding and following the coding region, e.g., 5′ untranslated (5′ UTR) or “leader” sequences and 3′ UTR or “trailer” sequences, as well as intervening sequences (intrans) between individual coding segments (exons). In some embodiments, a nucleic acid encoding a polypeptide as described herein (e.g., a CAR polypeptide) is comprised by a vector (e.g., a CAR vector). In some of the aspects described herein, a nucleic acid sequence encoding a given polypeptide as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” may refer to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example, in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

As used herein, the term “viral vector” may refer to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” may be a vector that includes a heterologous nucleic acid sequence or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, a “signal peptide” or “signal sequence” refers to a peptide at the N-terminus of a newly synthesized protein that serves to direct a nascent protein into the endoplasmic reticulum. In some embodiments, the signal peptide is a CD8 or lgK signal peptide.

As used herein the term “clustered regularly interspaced short palindromic repeats” or “CRISPR” may refer to a gene editing system that comprises a guide RNA component and a CRISPR associated (Cas) protein component. The guide RNA polynucleotide may comprise a homology region that is complementary to a target gene and a stem loop region that is capable of binding to a Cas protein. The Cas protein may comprise a guide RNA binding site and nuclease activity. The Cas protein and the guide RNA may for a complex that is capable of binding to the target gene (based on the homology region), and cleaving the DNA (using the nuclease activity of the Cas protein). In some embodiments, the Cas protein guide RNA complex binds to sequence that is adjacent to and downstream of a protospacer adjacent motif (PAM). Cleavage results in a DNA strand break and repair of that strand break may introduce a mutation (e.g., single nucleotide polymorphism, insertion, or deletion). In some embodiments, the Cas protein is any suitable Cas protein for mutating and/or altering the expression of a target gene (a Cas protein may also be referred to as a CRISPR protein herein). In some embodiments, the Cas protein is selected from the group consisting of a Cas9 protein, a Cas12 protein, or a Cas13 protein. The skilled person will understand that Cas proteins (e.g., Cas9) may have many different orthologs (e.g., SpyoCas9, spCas9, spyCas9, and geoCas9). In some embodiments, the Cas protein is SpyoCas9. Cas proteins and orthologs thereof are well known in the art as discussed in Gasiunas, Giedrius, et al, Nature communications 11.1 (2020): 1-10; and Fancheng Y et al., Cell Biology and Toxicology 35.6 (2019): 489-492, each of which is incorporated by reference in its entirety. Methods for designing guide RNAs (e.g., selecting homology region sequences for targeting a specific gene) are also well known in the art as described in Liu, Guanqing L. et al., Computational and Structural Biotechnology Journal 18 (2020): 35-44, which is incorporated by reference in its entirety. In some embodiments, gRNAs (encoded by gRNA polynucleotides) are designed using CRISPick (portals.broadinstitute.org/gppx/crispick/public), which performs as described in Doench et al., Nature Biotechnology, 34 (2), 184-191 (2016) and Sanson et al., Nature Communications, 9 (1), 5416 (2018), both of which are incorporated by reference in their entirety.

In some embodiments, a polypeptide, polynucleotide, plasmid and or/vector as described herein optionally further comprises a reporter molecule, e.g., to determine if the vector is properly expressed in a cell. In some embodiments, the reporter molecule may be a fluorescent protein (e.g., GFP, YFP, RF), antibody (e.g., CD34, tEGFR, tCD19, tCD20, tCD34, and tHer2), and a radioisotope. In some embodiments, the reporter molecule is hygromycin phosphotransferase (hph) that can be imaged alone or in combination with a substrate or chemical (for example 9-[4-[18F]fluoro-3-(hydroxymethyl)butyl]guanine ([18F]FHBG)).

In some embodiments, GFP and mCherry may be used as fluorescent tags for imaging a CAR expressed on a T cell (e.g., a CAR-T cell). It is expected that essentially any fluorescent protein known in the art can be used as a fluorescent tag for this purpose. For clinical applications, the CAR need not include a fluorescent tag or fluorescent protein. In each instance of particular constructs provided herein, therefore, any markers present in the constructs can be removed. The invention includes the constructs with or without the markers. Accordingly, when a specific construct is referenced herein, it can be considered with or without any markers or tags (including, e.g., histidine tags, such as the histidine tag of HHHHHH (SEQ ID NO: 1370)) as being included within the invention.

The term “statistically significant” or “significantly” refers to statistical significance and generally means a two standard deviation (2 SD) or greater difference.

Other than in the operating examples, or where otherwise indicated, all numbers expressing quantities of ingredients or reaction conditions used herein should be understood as modified in all instances by the term “about.”

The terms “about” or “approximately” when used in connection with a value can mean that the value or a statement reciting the value encompasses a range within ±1%, ±2%, ±3%, ±4%, ±5%, ±6%, ±7%, ±8%, ±9%, ±10%, ±1-5%, ±2-7%, ±3-8%, ±4-9%, or ±5-10% of the value.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology, as set forth herein.

### Guide RNA Polynucleotide Library

As used herein, the term “guide RNA (gRNA) polynucleotide” refers to a DNA or a RNA polynucleotide that encodes a guide RNA (gRNA). A guide RNA polynucleotide comprises a sequence that binds to a clustered regularly interspaced short palindromic repeats (CRISPR) protein or CRISPR-related protein and a sequence and comprises an additional sequence that is complementary to a target polynucleotide (i.e., a homology region). For example, a guide RNA polynucleotide may be a Cas9 protein guide RNA polynucleotide or a Cas12 protein guide RNA polynucleotide. Cas9 protein guide RNAs are compatible with Cas9 CRISPR proteins and are well known in the art e.g., as described in Adli et. al., Nature communications 9.1 (2018): 1-13, which is incorporated by reference in its entirety. Cas12 protein guide RNA polynucleotides are compatible with Cas12 CRISPR proteins and are well known in the art e.g., as described in Zetsche et al., Cell 163.3 (2015): 759-771, which is incorporated by reference in its entirety. In some embodiments, a guide RNA polynucleotide is a base editor guide RNA polynucleotide. In some embodiments, the gRNA is a prime editing guide RNA polynucleotide. In some embodiments, a guide RNA polynucleotide encodes a homology region (e.g., spacer) and a region that binds to a CRISPR protein (e.g., a direct repeat). In some embodiments, the guide RNA polynucleotide is a single guide RNA polynucleotide comprising a homology region and a region that binds to a CRISPR protein.

In some embodiments, the homology region comprises a sequential series of about 10-30 or about 15-25 nucleotides. In some embodiments, the homology region comprises about 20 nucleotides. In some embodiments, the homology region is complementary to a target gene (e.g., a gene associated with immune cell function). In some embodiments, the gRNAs are designed using an algorithm (e.g., CRISPick). CRISPick is described in Kim et al., Nat Biotechnology 36, 239-241 (2018); Doench et al. Nature Biotechnology, 34 (2), 184-191 (2016); and Sanson et al., Nature Communications, 9 (1), 5416 (2018), each of which are incorporated by reference in their entirety.

In some embodiments, the gRNA polynucleotides are not cross-reactive or minimally cross-reactive. Cross-reactive gRNA polynucleotides refer to guide RNA polynucleotides comprising homology regions having sufficient complementarity with more than one target polynucleotide (e.g., gene sequence) such that the gRNA may induce a CRISPR mutation in more than one target polynucleotide. Design algorithms may be used in guide RNA polynucleotide design to decrease the chances of cross-reactivity (e.g., gRNAs may be scored for on-target activity using Rule Set 3 (RS3) with sequence and target information and the Chen2013 tracr (Chen, Baohui, et al. Cell 155.7 (2013): 1479-1491). gRNAs may also be scored for off-target activity using Tier-agnostic 1 mismatch aggregated Cutting Frequency Determination (CFD) scores). The skilled person will understand the gRNA polynucleotides designed with such an algorithm may still have some degree of cross-reactivity, however, the risk of cross reactivity is expected to be decreased or may be specified at a selected threshold in the algorithm. In some embodiments, cross reactivity is a function of complementarity. In some embodiments, gRNA polynucleotides that are not cross-reactive do not have greater than 80% complementarity to more than 1 gene. In some embodiments, gRNA polynucleotides that are not cross-reactive do not have greater than 85% complementarity to more than 1 gene. In some embodiments, gRNA polynucleotides that are not cross-reactive do not have greater than 90% complementarity to more than 1 gene. In some embodiments, gRNA polynucleotides that are not cross-reactive do not have greater than 95% complementarity to more than 1 gene.

The term “complementary” as described herein refers to the degree of Watson-Crick base pairing between two polynucleotides. For example, two polynucleotides may be 90% complementary if 9/10 nucleotides of each of the polynucleotides form a Watson Crick base pair. In some embodiments, complementary may refer to at least 70% (e.g., at least 70%, at least 80%, at least 90%, at least 95%, or at least 99%) of nucleotides in a first polynucleotide Watson-Crick base pairing with a second polynucleotide. In some embodiments, a homology region of a gRNA is complementary to a gene sequence when the homology region is capable of hybridizing to the gene sequence and at least a threshold percentage (e.g., at least 70% (e.g., at least 70%, at least 80%, at least 90%, at least 95%, or at least 99%) of nucleotides are Watson Crick base pairs to the gene sequence. In some embodiments, a homology region is complementary to a gene sequence when the homology region is capable of hybridizing to the gene sequence and initiating cleavage of the gene sequence by a CRISPR protein and at least a threshold percentage (e.g., at least 70% (e.g., at least 70%, at least 80%, at least 90%, at least 95%, or at least 99%) of nucleotides are Watson Crick base pairs to the gene sequence. In some embodiments, a homology region is complementary to a target gene sequence when the nucleotides of the homology region are 100% complementary to a sequential portion of the target gene sequence (e.g., 10, 11, 12, 13, 14, 15, 16, 17, 18, 19, 20, 21, 22, 23, 24, 25, 26, 27, 28, 29, or 30 sequential nucleotides of the target gene sequence, e.g., 20 sequential nucleotides). In some embodiments, the homology region is complementary to the sense strand of the target gene sequence. In some embodiments, the homology region is complementary to the anti-sense strand of the target gene sequence. In some embodiments, a homology region that is complementary to a gene encoded by a sequence (e.g., SEQ ID NO: 1) may refer to a homology region that is complementary to either the sense strand of the gene sequence (e.g., SEQ ID NO: 1) or the antisense strand of the gene sequence (e.g., the reverse compliment of SEQ ID NO: 1). For example, a homology region described herein may be complementary to any one of SEQ ID NOs: 1-135 (i.e. a sense strands) or the reverse complement of any one of SEQ ID NOs: 1-135 (i.e., antisense strands).

In some embodiments, the homology region is complementary to a region of the target gene sequence this is adjacent to a protospacer adjacent motif (PAM). In some embodiments, the homology region is complementary to a region of the target gene sequence that is downstream of and adjacent to a protospacer adjacent motif (PAM).

In some embodiments, this disclosure describes a composition comprising a plurality of guide RNA (gRNA) polynucleotides, wherein at least 2 (e.g., at least 3, at least 5, at least 10, at least 15, at least 25, at least 50, at least 75, at least 100, or at least 135) gRNA polynucleotides each comprise a homology region that is complementary to a sense strand or an antisense strand of a different gene encoded by a sequence of any one of SEQ ID NOs: 1-135 or a variant thereof.

In some embodiments, this disclosure describes a composition comprising a plurality of guide RNA (gRNA) polynucleotides, wherein at least 2 (e.g., at least 3, at least 5, at least 10, at least 15, at least 25, at least 50, at least 75, at least 100, or at least 135) gRNA polynucleotides each comprise a homology region that is complementary to a sense strand or an antisense strand of a different gene sequence of any one of SEQ ID NOs: 1-135 or a variant thereof.

In some embodiments, this disclosure describes a composition comprising a plurality of guide RNA (gRNA) polynucleotides, wherein at least 2 (e.g., at least 3, at least 5, at least 10, at least 15, at least 25, at least 50, at least 75, at least 100, or at least 135) gRNA polynucleotides each comprise a homology region that is complementary to a different gene sequence of any one of SEQ ID NOs: 1-135 or a variant thereof, or wherein the at least 2 (e.g., at least 3, at least 5, at least 10, at least 15, at least 25, at least 50, at least 75, at least 100, or at least 135) gRNA polynucleotides each comprise a homology region that is complementary to a different gene sequence selected from a reverse complement of any one of SEQ ID NOs: 1-135.

In some embodiments, the instant disclosure describes a compositions comprising a plurality of guide RNA (gRNA) polynucleotides. In some embodiments, at least 2 gRNA polynucleotides e.g., each comprise a homology region complementary to a different gene sequence of any one of SEQ ID NOs: 1-135 or a variant thereof. For example, a plurality of guide RNA (gRNA) polynucleotides can comprise a first gRNA polynucleotide and a second gRNA polynucleotide, wherein the first gRNA polynucleotide and the second gRNA polynucleotide are each complementary to a different gene sequence of any one of SEQ ID NOs: 1-135 or a variant thereof. In some embodiments, each homology region of the at least 2 gRNA polynucleotides is complementary to a sense strand of a different gene sequence of any one of SEQ ID NOs: 1-135 or a variant thereof. In some embodiments, each homology region of the at least 2 gRNA polynucleotides is complementary to an antisense strand of a different gene sequence of any one of SEQ ID NOs: 1-135 or a variant thereof.

In some embodiments, the composition comprises at least 2 gRNA polynucleotides, wherein each gRNA polynucleotide comprises a homology region complementary to a different gene sequence of any one of SEQ ID NOs: 1-135, or a variant thereof. In some embodiments, the composition comprises at least 3 gRNA polynucleotides, wherein each gRNA polynucleotide comprises a homology region complementary to a different gene sequence of any one of SEQ ID NOs: 1-135, or a variant thereof. In some embodiments, the composition comprises at least 4 gRNA polynucleotides, wherein each gRNA polynucleotide comprises a homology region complementary to a different gene sequence of any one of SEQ ID NOs: 1-135, or a variant thereof. In some embodiments, the composition comprises at least 5 gRNA polynucleotides. In some embodiments, the composition comprises at least 6 gRNA polynucleotides, wherein each gRNA polynucleotide comprises a homology region complementary to a different gene sequence of any one of SEQ ID NOs: 1-135, or a variant thereof. In some embodiments, the composition comprises at least 7 gRNA polynucleotides, wherein each gRNA polynucleotide comprises a homology region complementary to a different gene sequence of any one of SEQ ID NOs: 1-135, or a variant thereof. In some embodiments, the composition comprises at least 8 gRNA polynucleotides, wherein each gRNA polynucleotide comprises a homology region complementary to a different gene sequence of any one of SEQ ID NOs: 1-135, or a variant thereof. In some embodiments, the composition comprises at least 9 gRNA polynucleotides, wherein each gRNA polynucleotide comprises a homology region complementary to a different gene sequence of any one of SEQ ID NOs: 1-135, or a variant thereof. In some embodiments, the composition comprises at least 10 gRNA polynucleotides, wherein each gRNA polynucleotide comprises a homology region complementary to a different gene sequence of any one of SEQ ID NOs: 1-135, or a variant thereof. In some embodiments, the composition comprises at least 11 gRNA polynucleotides, wherein each gRNA polynucleotide comprises a homology region complementary to a different gene sequence of any one of SEQ ID NOs: 1-135, or a variant thereof. In some embodiments, the composition comprises at least 12 gRNA polynucleotides, wherein each gRNA polynucleotide comprises a homology region complementary to a different gene sequence of any one of SEQ ID NOs: 1-135, or a variant thereof. In some embodiments, the composition comprises at least 13 gRNA polynucleotides, wherein each gRNA polynucleotide comprises a homology region complementary to a different gene sequence of any one of SEQ ID NOs: 1-135, or a variant thereof. In some embodiments, the composition comprises at least 14 gRNA polynucleotides, wherein each gRNA polynucleotide comprises a homology region complementary to a different gene sequence of any one of SEQ ID NOs: 1-135, or a variant thereof. In some embodiments, the composition comprises at least 15 gRNA polynucleotides, wherein each gRNA polynucleotide comprises a homology region complementary to a different gene sequence of any one of SEQ ID NOs: 1-135, or a variant thereof. In some embodiments, the composition comprises at least 16 gRNA polynucleotides, wherein each gRNA polynucleotide comprises a homology region complementary to a different gene sequence of any one of SEQ ID NOs: 1-135, or a variant thereof. In some embodiments, the composition comprises at least 17 gRNA polynucleotides, wherein each gRNA polynucleotide comprises a homology region complementary to a different gene sequence of any one of SEQ ID NOs: 1-135, or a variant thereof. In some embodiments, the composition comprises at least 18 gRNA polynucleotides, wherein each gRNA polynucleotide comprises a homology region complementary to a different gene sequence of any one of SEQ ID NOs: 1-135, or a variant thereof. In some embodiments, the composition comprises at least 19 gRNA polynucleotides, wherein each gRNA polynucleotide comprises a homology region complementary to a different gene sequence of any one of SEQ ID NOs: 1-135, or a variant thereof. In some embodiments, the composition comprises at least 20 gRNA polynucleotides, wherein each gRNA polynucleotide comprises a homology region complementary to a different gene sequence of any one of SEQ ID NOs: 1-135, or a variant thereof. In some embodiments, the composition comprises at least 21 gRNA polynucleotides, wherein each gRNA polynucleotide comprises a homology region complementary to a different gene sequence of any one of SEQ ID NOs: 1-135, or a variant thereof. In some embodiments, the composition comprises at least 22 gRNA polynucleotides, wherein each gRNA polynucleotide comprises a homology region complementary to a different gene sequence of any one of SEQ ID NOs: 1-135, or a variant thereof. In some embodiments, the composition comprises at least 23 gRNA polynucleotides, wherein each gRNA polynucleotide comprises a homology region complementary to a different gene sequence of any one of SEQ ID NOs: 1-135, or a variant thereof. In some embodiments, the composition comprises at least 24 gRNA polynucleotides, wherein each gRNA polynucleotide comprises a homology region complementary to a different gene sequence of any one of SEQ ID NOs: 1-135, or a variant thereof. In some embodiments, the composition comprises at least 25 gRNA polynucleotides, wherein each gRNA polynucleotide comprises a homology region complementary to a different gene sequence of any one of SEQ ID NOs: 1-135, or a variant thereof. In some embodiments, the composition comprises at least 26 gRNA polynucleotides, wherein each gRNA polynucleotide comprises a homology region complementary to a different gene sequence of any one of SEQ ID NOs: 1-135, or a variant thereof. In some embodiments, the composition comprises at least 27 gRNA polynucleotides, wherein each gRNA polynucleotide comprises a homology region complementary to a different gene sequence of any one of SEQ ID NOs: 1-135, or a variant thereof. In some embodiments, the composition comprises at least 28 gRNA polynucleotides, wherein each gRNA polynucleotide comprises a homology region complementary to a different gene sequence of any one of SEQ ID NOs: 1-135, or a variant thereof. In some embodiments, the composition comprises at least 29 gRNA polynucleotides, wherein each gRNA polynucleotide comprises a homology region complementary to a different gene sequence of any one of SEQ ID NOs: 1-135, or a variant thereof. In some embodiments, the composition comprises at least 30 gRNA polynucleotides, wherein each gRNA polynucleotide comprises a homology region complementary to a different gene sequence of any one of SEQ ID NOs: 1-135, or a variant thereof. In some embodiments, the composition comprises at least 40 gRNA polynucleotides, wherein each gRNA polynucleotide comprises a homology region complementary to a different gene sequence of any one of SEQ ID NOs: 1-135, or a variant thereof. In some embodiments, the composition comprises at least 50 gRNA polynucleotides, wherein each gRNA polynucleotide comprises a homology region complementary to a different gene sequence of any one of SEQ ID NOs: 1-135, or a variant thereof. In some embodiments, the composition comprises at least 60 gRNA polynucleotides, wherein each gRNA polynucleotide comprises a homology region complementary to a different gene sequence of any one of SEQ ID NOs: 1-135, or a variant thereof. In some embodiments, the composition comprises at least 70 gRNA polynucleotides, wherein each gRNA polynucleotide comprises a homology region complementary to a different gene sequence of any one of SEQ ID NOs: 1-135, or a variant thereof. In some embodiments, the composition comprises at least 80 gRNA polynucleotides, wherein each gRNA polynucleotide comprises a homology region complementary to a different gene sequence of any one of SEQ ID NOs: 1-135, or a variant thereof. In some embodiments, the composition comprises at least 90 gRNA polynucleotides, wherein each gRNA polynucleotide comprises a homology region complementary to a different gene sequence of any one of SEQ ID NOs: 1-135, or a variant thereof. In some embodiments, the composition comprises at least 100 gRNA polynucleotides, wherein each gRNA polynucleotide comprises a homology region complementary to a different gene sequence of any one of SEQ ID NOs: 1-135, or a variant thereof. In some embodiments, the composition comprises at least 110 gRNA polynucleotides, wherein each gRNA polynucleotide comprises a homology region complementary to a different gene sequence of any one of SEQ ID NOs: 1-135, or a variant thereof. In some embodiments, the composition comprises at least 120 gRNA polynucleotides, wherein each gRNA polynucleotide comprises a homology region complementary to a different gene sequence of any one of SEQ ID NOs: 1-135, or a variant thereof. In some embodiments, the composition comprises at least 130 gRNA polynucleotides), wherein each gRNA polynucleotide comprises a homology region complementary to a different gene sequence of any one of SEQ ID NOs: 1-135, or a variant thereof.

In some embodiment, a plurality of guide RNA polynucleotides comprises at least 2 guide RNA polynucleotides (e.g., at least 2, at least 5, at least 25, at least 50, at least 100, at least 250, at least 500, at least 1000, at least 2000, at least 2500, at least 5000, at least 10000, at least 15000, at least 20000, at least 25000, at least 30000, at least 50000, at least 100000, or at least 1000000 guide RNA polynucleotides) having different sequences. In some embodiments, a plurality of plurality of gRNA polynucleotides comprises no more than 1000, 2500, 5000, 10,000, 15,000 or 20,000 polynucleotides. In some embodiments, the plurality of guide RNA polynucleotides comprises between 2-1000, 2-2500, 2-5000, 2-1000, 2-15000, 2-20000, 2-40000, 2-50000, 2-100000, 5-1000, 5-2500, 5-5000, 5-1000, 5-15000, 5-20000, 5-40000, 5-50000, 5-100000, 10-1000, 10-2500, 10-5000, 10-1000, 10-15000, 10-20000, 10-40000, 10-50000, 10-100000, 20-1000, 20-2500, 20-5000, 20-1000, 20-15000, 20-20000, 20-40000, 20-50000, 20-100000, 50-1000, 50-2500, 50-5000, 50-10000, 50-15000, 50-20000, 50-40000, 50-50000, 50-100000, 100-1000, 100-2500, 100-5000, 100-10000, 100-15000, 100-20000, 100-40000, 100-50000, 100-100000, 135-1000, 135-2500, 135-5000, 135-10000, 135-15000, 135-20000, 135-40000, 135-50000, or 135-100000 guide RNA polynucleotides having different sequences. In some embodiments, the different sequences are complementary to one or more of SEQ ID NOs: 1-135. In some embodiments, the plurality of guide RNA polynucleotides comprises between 2-1300 guide RNA polynucleotides having different sequences, In some embodiments, the plurality of guide RNA polynucleotides comprises between 2-1200 guide RNA polynucleotides having different sequences, In some embodiments, the plurality of guide RNA polynucleotides comprises between 2-1100 guide RNA polynucleotides having different sequences, In some embodiments, the plurality of guide RNA polynucleotides comprises between 2-1080 guide RNA polynucleotides having different sequences, In some embodiments, the plurality of guide RNA polynucleotides comprises between 5-1300 guide RNA polynucleotides having different sequences, In some embodiments, the plurality of guide RNA polynucleotides comprises between 5-1200 guide RNA polynucleotides having different sequences, In some embodiments, the plurality of guide RNA polynucleotides comprises between 5-1100 guide RNA polynucleotides having different sequences, In some embodiments, the plurality of guide RNA polynucleotides comprises between 5-1080 guide RNA polynucleotides having different sequences, In some embodiments, the plurality of guide RNA polynucleotides comprises between 10-1300 guide RNA polynucleotides having different sequences, In some embodiments, the plurality of guide RNA polynucleotides comprises between 10-1200 guide RNA polynucleotides having different sequences, In some embodiments, the plurality of guide RNA polynucleotides comprises between 10-1100 guide RNA polynucleotides having different sequences, In some embodiments, the plurality of guide RNA polynucleotides comprises between 10-1080 guide RNA polynucleotides having different sequences,

In some embodiments, the composition comprises at most 1000 (e.g., at most 2500, at most 5000, at most 7500, at most 10000, at most 15000, at most 20000, at most 25000, at most 30000, at most 35000, at most 40000, at most 45000, at most 50000, at most 60000, at most 70000, at most 80000, at most 90000, at most 100000 polynucleotides, at most 250,000, at most 500,000, at most 750,000 or at most 1,000,000) guide RNA polynucleotides having different sequences. In some embodiments, the composition comprises at most 1000 guide RNA polynucleotides having different sequences. In some embodiments, the composition comprises at most 2500 guide RNA polynucleotides having different sequences. In some embodiments, the composition comprises at most 5000 guide RNA polynucleotides having different sequences, In some embodiments, the composition comprises at most 7500 guide RNA polynucleotides having different sequences. In some embodiments, the composition comprises at most 10000 guide RNA polynucleotides having different sequences. In some embodiments, the composition comprises at most 15000 guide RNA polynucleotides having different sequences. In some embodiments, the composition comprises at most 20000 guide RNA polynucleotides having different sequences. In some embodiments, the compositions comprise at most 25000 guide RNA polynucleotides having different sequences. In some embodiments, the composition comprises at most 30000 guide RNA polynucleotides having different sequences. In some embodiments, the composition comprises at most 35000 guide RNA polynucleotides having different sequences. In some embodiments, the composition comprises at most 40000 guide RNA polynucleotides having different sequences. In some embodiments, the composition comprises at most 45000 guide RNA polynucleotides having different sequences. In some embodiments, the composition comprises at most 50000 guide RNA polynucleotides having different sequences. In some embodiments, the composition comprises at most 60000 guide RNA polynucleotides having different sequences. In some embodiments, the composition comprises at most 70000 guide RNA polynucleotides having different sequences. In some embodiments, the composition comprises at most 80000 guide RNA polynucleotides having different sequences. In some embodiments, the composition comprises at most 90000 guide RNA polynucleotides having different sequences. In some embodiments, the composition comprises at most 100000 guide RNA polynucleotides having different sequences. In some embodiments, the compositions comprises between 2-1000, 2-2500, 2-5000, 2-1000, 2-15000, 2-20000, 2-40000, 2-50000, 2-100000, 2-1000000, 5-1000, 5-2500, 5-5000, 5-1000, 5-15000, 5-20000, 5-40000, 5-50000, 5-100000, 5-1000000, 10-1000, 10-2500, 10-5000, 10-1000, 10-15000, 10-20000, 10-40000, 10-50000, 10-100000, 10-1000000, 20-1000, 20-2500, 20-5000, 20-1000, 20-15000, 20-20000, 20-40000, 20-50000, 20-100000, 20-1000000, 50-1000, 50-2500, 50-5000, 50-10000, 50-15000, 50-20000, 50-40000, 50-50000, 50-100000, 50-1000000, 100-1000, 100-2500, 100-5000, 100-10000, 100-15000, 100-20000, 100-40000, 100-50000, 100-100000, 100-1000000, 135-1000, 135-2500, 135-5000, 135-10000, 135-15000, 135-20000, 135-40000, 135-50000, or 135-100000, 135-1000000, 1080-1000, 1080-2500, 1080-5000, 1080-10000, 1080-15000, 1080-20000, 1080-40000, 1080-50000, or 1080-100000, 1080-1000000 guide RNA polynucleotides having different sequences. The skilled person will understand that many copies of any one guide RNA polynucleotide may be present in the composition and that these copies have the same sequence.

In some embodiments, the guide RNA polynucleotides of the composition are complementary to at most 20,000 different gene sequences. In some embodiments, the guide RNA polynucleotides of the composition are complementary to at most 19,000 different gene sequences. In some embodiments, the guide RNA polynucleotides of the composition are complementary to at most 18,000 different gene sequences. In some embodiments, the guide RNA polynucleotides of the composition are complementary to at most 17,000 different gene sequences. In some embodiments, the guide RNA polynucleotides of the composition are complementary to at most 16,000 different gene sequences. In some embodiments, the guide RNA polynucleotides of the composition are complementary to at most 15,000 different gene sequences. In some embodiments, the guide RNA polynucleotides of the composition are complementary to at most 14,000 different gene sequences. In some embodiments, the guide RNA polynucleotides of the composition are complementary to at most 13,000 different gene sequences. In some embodiments, the guide RNA polynucleotides of the composition are complementary to at most 12,000 different gene sequences. In some embodiments, the guide RNA polynucleotides of the composition are complementary to at most 11,000 different gene sequences. In some embodiments, the guide RNA polynucleotides of the composition are complementary to at most 10,000 different gene sequences. In some embodiments, the guide RNA polynucleotides of the composition are complementary to at most 9,000 different gene sequences. In some embodiments, the guide RNA polynucleotides of the composition are complementary to at most 8,000 different gene sequences. In some embodiments, the guide RNA polynucleotides of the composition are complementary to at most 7,000 different gene sequences. In some embodiments, the guide RNA polynucleotides of the composition are complementary to at most 6,000 different gene sequences. In some embodiments, the guide RNA polynucleotides of the composition are complementary to at most 5,000 different gene sequences. In some embodiments, the guide RNA polynucleotides of the composition are complementary to at most 4,000 different gene sequences. In some embodiments, the guide RNA polynucleotides of the composition are complementary to at most 3,000 different gene sequences. In some embodiments, the guide RNA polynucleotides of the composition are complementary to at most 2,000 different gene sequences. In some embodiments, the guide RNA polynucleotides of the composition are complementary to at most 1,000 different gene sequences. In some embodiments, the guide RNA polynucleotides of the composition are complementary to at most 900 different gene sequences. In some embodiments, the guide RNA polynucleotides of the composition are complementary to at most 800 different gene sequences.

In some embodiments, the guide RNA polynucleotides of the composition are complementary to at most 700 different gene sequences. In some embodiments, the guide RNA polynucleotides of the composition are complementary to at most 600 different gene sequences. In some embodiments, the guide RNA polynucleotides of the composition are complementary to at most 500 different gene sequences. In some embodiments, the guide RNA polynucleotides of the composition are complementary to at most 400 different gene sequences. In some embodiments, the guide RNA polynucleotides of the composition are complementary to at most 300 different gene sequences. In some embodiments, the guide RNA polynucleotides of the composition are complementary to at most 200 different gene sequences. In some embodiments, the guide RNA polynucleotides of the composition are complementary to at most 135 different gene sequences.

In some embodiments, the guide RNA polynucleotides of the composition are complementary 135 different gene sequences (e.g., SEQ ID NOs: 1-135).

In some embodiments, the guide RNAs of the composition do not comprise a guide RNA polynucleotide that is complementary to a sense strand or an antisense strand of a gene encoding a tumor suppressor. Without being bound to theory, mutation (e.g., loss of function) of a tumor suppressor gene in a cell (e.g., a CAR-T cell) can result in the cell becoming tumorigenic or cancerous. In some embodiments, this is undesirable in the guide RNA polynucleotide compositions described herein because these compositions may be used to generate a cellular therapy (e.g., CAR-T cell therapy) that is administered to a subject (e.g., a human subject having cancer). If the guide RNA polynucleotide library comprises a guide RNA that is complementary to a tumor suppressor, this may generate a cell (e.g. CAR T cell) that could give a subject cancer or a tumor. Genes that encode tumor suppressors are known in the art e.g., as described in Cooper G M. The Cell: A Molecular Approach. 2nd edition. Sunderland (MA): Sinauer Associates; 2000. Tumor Suppressor Genes. Available from: ncbi.nlm.nih.gov/books/NBK9894/.

In some embodiments, the compositions (e.g., compositions comprising a plurality of gRNA polynucleotides) described herein do not comprises on one or more of p53 (e.g., e.g., Uniprot: P04637), Rb (e.g., Uniprot: P06400), PTEN (e.g., Uniprot: P60484), BRCA1 (e.g., Uniprot: P38398), and/or BRCA2 (e.g., Uniprot: P51587). In some embodiments, the compositions (e.g., compositions comprising a plurality of gRNA polynucleotides) described herein do not comprises on one or more of P53 (e.g., Uniprot: P04637), PTEN (e.g., Uniprot: P60484), APC (e.g., Uniprot: P25054), P16INK4a (e.g., Uniprot: P42771), P15INK4b (e.g., Uniprot: P42772), Cadherin-1 (e.g., Uniprot: Q12864), RB1 (e.g., Uniprot: P06400), BRCA1 (e.g., Uniprot: P38398), Wilms tumor 1 (e.g., Uniprot: P19544), STK11 (e.g., Uniprot: Q15831), Smad4 (e.g., Uniprot: Q13485), BRCA2 (e.g., Uniprot: P51587), CHEK2 (e.g., Uniprot: O96017), P14arf (e.g., Uniprot: Q8N726), P21 (e.g., Uniprot: Q9H633), P73 (e.g., Uniprot: O15350), PTCH1 (e.g., Uniprot: Q13635), and/or MSH2 (e.g., Uniprot: P43246).

In some embodiments, the guide RNAs of the composition do not comprise a guide RNA polynucleotide that is complementary to a sense strand or an antisense strand of a gene that is essential for T cell function. In some embodiments, the guide RNAs of the composition do not comprise a guide RNA polynucleotide that is complementary to a sense strand or an antisense strand of a gene encoding of one or more of DAD1 (e.g., Uniprot: P61803), SUPT4H1 (e.g., Uniprot: P63272), ZNF626 (e.g., Uniprot: Q68DY1), RAC2 (e.g., Uniprot: P15153), MARS (e.g., Uniprot: P56192), NAA10 (e.g., Uniprot: P41227), ORAOV1 (e.g., Uniprot: Q8WV07), TRMT112 (e.g., Uniprot: Q9UI30), RPP21 (e.g., Uniprot: Q9H633), VHL (e.g., Uniprot: P40337), NOP14 (e.g., Uniprot: P78316), OGT (e.g., Uniprot: O15294), UTP3 (e.g., Uniprot: Q9NQZ2), VARS (e.g., Uniprot: P26640), CCND3 (e.g., Uniprot: P30281), TAF6 (e.g., Uniprot: P49848), CDK6 (e.g., Uniprot: Q00534), CD247 (e.g., Uniprot: P20963), LCP2 (e.g., Uniprot: Q13094), EXOSC6 (e.g., Uniprot: Q5RKV6), RHOH (e.g., Uniprot: Q15669), VAV1 (e.g., Uniprot: P15498), MYC (e.g., Uniprot: P01106), POLR3H (e.g., Uniprot: Q9Y535), LAT (e.g., Uniprot: 043561), CD3D (e.g., Uniprot: P04234), WDR18 (e.g., Uniprot: Q9BV38), POLR2L (e.g., Uniprot: P62875), TMX1 (e.g., Uniprot: Q9H3N1), PRF1 (e.g., Uniprot: Q42449), STX11 (e.g., Uniprot: O75558), or STXBP2 (e.g., Uniprot: Q15833), or a variant thereof.

In some embodiments, a variant thereof refers to a homolog, ortholog or paralog of a gene (e.g., a gene encoded by any one of SEQ ID NOs: 1-135). In some embodiments, variants of the same gene are different alleles. In some embodiments, a variant thereof refers to an amino acid sequence that is at least 60% identical to any one of SEQ ID NOs: 1-135. In some embodiments, a variant thereof refers to an amino acid sequence that is at least 65% identical to any one of SEQ ID NOs: 1-135. In some embodiments, a variant thereof refers to an amino acid sequence that is at least 70% identical to any one of SEQ ID NOs: 1-135. In some embodiments, a variant thereof refers to an amino acid sequence that is at least 75% identical to any one of SEQ ID NOs: 1-135. In some embodiments, a variant thereof refers to an amino acid sequence that is at least 80% identical to any one of SEQ ID NOs: 1-135. In some embodiments, a variant thereof refers to an amino acid sequence that is at least 85% identical to any one of SEQ ID NOs: 1-135. In some embodiments, a variant thereof refers to an amino acid sequence that is at least 90% identical to any one of SEQ ID NOs: 1-135. In some embodiments, a variant thereof refers to an amino acid sequence that is at least 95% identical to any one of SEQ ID NOs: 1-135. In some embodiments, a variant thereof refers to an amino acid sequence that is at least 99% identical to any one of SEQ ID NOs: 1-135. In some embodiments, a variant thereof refers to an amino acid sequence that is at least 99.5% identical to any one of SEQ ID NOs: 1-135. In some embodiments, a variant thereof refers to an amino acid sequence that is at least 99.9% identical to any one of SEQ ID NOs: 1-135.

In some embodiments, the composition comprises at least X gRNA polynucleotides per gene sequence, wherein the at least X gRNA polynucleotides comprise different sequences complementary to a given gene. For example, in some embodiments, the composition comprises at least 2 gRNA polynucleotides per gene sequence, wherein the at least 2 gRNA polynucleotides comprise different sequences complementary to a given gene (e.g., the sense strand or antisense strand of the gene). So in such embodiments, if the composition comprises gRNA polynucleotides comprising homology regions complementary to Y different genes (e.g., any one of Y different SEQ ID NOs), then the composition would comprise at least 2Y gRNA polynucleotides, with at least 2 gRNA polynucleotides comprising homology regions complementary to each of the Y genes and wherein those at least 2 gRNA polynucleotides comprise different sequences complementary to a given gene. In some embodiments, the at least X gRNA polynucleotides comprise homology regions complementary to different regions (e.g., different exons or introns or different regions encoding different domains of the protein encoded by a gene) of the same gene sequence. In some embodiments, the at least X gRNA polynucleotides comprise homology regions complementary to overlapping regions of the same gene sequence. In some embodiments, the composition comprises at least 2 gRNA polynucleotides per gene sequence, wherein the gRNA polynucleotides comprise different sequences complementary to said gene sequence (e.g., SEQ ID NOs: 1-135).

In some embodiments, the composition comprises at least 2 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 3 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 4 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 5 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 6 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 7 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 8 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 9 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 10 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 12 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 15 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 18 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135). In some embodiments, the composition comprises at least 20 gRNA polynucleotides per gene sequence, wherein the gRNA polynucleotides comprise different sequences complementary to said gene sequence (e.g., SEQ ID NOs: 1-135, or the reverse complement of any one of SEQ ID NOs: 1-135).

In some embodiments, the composition comprises 1-20, 2-20, 2-15, 2-10, 2-8, 2-5, 4-20, 4-15, 4-10, 4-8, 3-15, 3-10, 3-8, 3-6, 4-20, 4-15, 4-10, 4-8, 4-6, 5-20, 5-15, 5-10, 5-8, 6-20, 6-15, 6-10, 6-8, 7-20, 7-15, 7-10, or 7-8 polynucleotides per gene sequence, wherein each gRNA polynucleotide comprises different sequences complementary to said gene sequence or the reverse complement of said gene sequence. In some embodiments, the composition comprises 1, 2, 3, 4, 5, 6, 7, 8, 9, 10, 11, 12, 13, 14, 15, 16, 17, 18, 19, 20, or more gRNA polynucleotides per gene sequence, wherein each gRNA polynucleotide comprises a different sequence complementary to said gene sequence or the reverse complement of said gene sequence. In some embodiments, the composition comprises 8 gRNA polynucleotides per gene sequence, wherein each gRNA polynucleotide comprises a different sequence complementary to said gene sequence or the reverse complement of said gene sequence. In some embodiments, the multiple gRNA polynucleotides (e.g., 8) are complementary to different regions of the same gene sequence or the reverse complement of said gene sequence.

In some embodiments, the composition comprises one or more negative control guide RNA polynucleotides. In some embodiments, a negative control guide RNA polynucleotide is a guide RNA polynucleotide that is not expected to have an effect of CAR-T cell efficacy. In some embodiments, a negative control guide RNA polynucleotide is a guide RNA polynucleotide whose homology region is not complementary to a gene in the genome of the immune cell (e.g., CAR-T cell). In some embodiments, a negative control gRNA comprises a homology regions that is complementary intergenic regions. In some embodiments, a negative control gRNA polynucleotide comprises a randomly generated homology region. In some embodiments, negative control guide RNA sequences comprise nucleotides sequence of any one of SEQ ID NOs: 1216-1315.

In some embodiments, the composition comprises at least 1 gRNA polynucleotide of any one of SEQ ID NOs: 136-1215 or any variant thereof. In some embodiments, the composition comprises at least 2 gRNA polynucleotide of any one of SEQ ID NOs: 136-1215 or a variant of any thereof. In some embodiments, the composition comprises at least 3 gRNA polynucleotide of any one of SEQ ID NOs: 136-1215 or any variant thereof. In some embodiments, the composition comprises at least 4 gRNA polynucleotide of any one of SEQ ID NOs: 136-1215 or any variant thereof. In some embodiments, the composition comprises at least 5 gRNA polynucleotide of any one of SEQ ID NOs: 136-1215 or any variant thereof. In some embodiments, the composition comprises at least 6 gRNA polynucleotide of any one of SEQ ID NOs: 136-1215 or any variant thereof. In some embodiments, the composition comprises at least 7 gRNA polynucleotide of any one of SEQ ID NOs: 136-1215 or any variant thereof. In some embodiments, the composition comprises at least 8 gRNA polynucleotide of any one of SEQ ID NOs: 136-1215 or any variant thereof. In some embodiments, the composition comprises at least 9 gRNA polynucleotide of any one of SEQ ID NOs: 136-1215 or any variant thereof. In some embodiments, the composition comprises at least 10 gRNA polynucleotide of any one of SEQ ID NOs: 136-1215 or any variant thereof. In some embodiments, the composition comprises at least 11 gRNA polynucleotide of any one of SEQ ID NOs: 136-1215 or any variant thereof. In some embodiments, the composition comprises at least 12 gRNA polynucleotide of any one of SEQ ID NOs: 136-1215 or any variant thereof. In some embodiments, the composition comprises at least 13 gRNA polynucleotide of any one of SEQ ID NOs: 136-1215 or any variant thereof. In some embodiments, the composition comprises at least 14 gRNA polynucleotide of any one of SEQ ID NOs: 136-1215 or any variant thereof. In some embodiments, the composition comprises at least 15 gRNA polynucleotide of any one of SEQ ID NOs: 136-1215 or any variant thereof. In some embodiments, the composition comprises at least 16 gRNA polynucleotide of any one of SEQ ID NOs: 136-1215 or any variant thereof. In some embodiments, the composition comprises at least 17 gRNA polynucleotide of any one of SEQ ID NOs: 136-1215 or any variant thereof. In some embodiments, the composition comprises at least 18 gRNA polynucleotide of any one of SEQ ID NOs: 136-1215 or any variant thereof. In some embodiments, the composition comprises at least 19 gRNA polynucleotide of any one of SEQ ID NOs: 136-1215 or any variant thereof. In some embodiments, the composition comprises at least 20 gRNA polynucleotide of any one of SEQ ID NOs: 136-1215 or any variant thereof. In some embodiments, the composition comprises at least 21 gRNA polynucleotide of any one of SEQ ID NOs: 136-1215 or any variant thereof. In some embodiments, the composition comprises at least 22 gRNA polynucleotide of any one of SEQ ID NOs: 136-1215 or any variant thereof. In some embodiments, the composition comprises at least 23 gRNA polynucleotide of any one of SEQ ID NOs: 136-1215 or any variant thereof. In some embodiments, the composition comprises at least 24 gRNA polynucleotide of any one of SEQ ID NOs: 136-1215 or any variant thereof. In some embodiments, the composition comprises at least 25 gRNA polynucleotide of any one of SEQ ID NOs: 136-1215 or any variant thereof. In some embodiments, the composition comprises at least 26 gRNA polynucleotide of any one of SEQ ID NOs: 136-1215 or any variant thereof. In some embodiments, the composition comprises at least 27 gRNA polynucleotide of any one of SEQ ID NOs: 136-1215 or any variant thereof. In some embodiments, the composition comprises at least 28 gRNA polynucleotide of any one of SEQ ID NOs: 136-1215 or any variant thereof. In some embodiments, the composition comprises at least 29 gRNA polynucleotide of any one of SEQ ID NOs: 136-1215 or any variant thereof. In some embodiments, the composition comprises at least 30 gRNA polynucleotide of any one of SEQ ID NOs: 136-1215 or any variant thereof. In some embodiments, the compositions comprise at least 40 gRNA polynucleotide of any one of SEQ ID NOs: 136-1215 or any variant thereof. In some embodiments, the composition comprises at least 50 gRNA polynucleotide of any one of SEQ ID NOs: 136-1215 or any variant thereof. In some embodiments, the compositions comprises at least 60 gRNA polynucleotide of any one of SEQ ID NOs: 136-1215 or any variant thereof.

In some embodiments, the composition comprises at least 70 gRNA polynucleotides of any one of SEQ ID NOs: 136-1215 or any variant thereof. In some embodiments, the composition comprises at least 80 gRNA polynucleotides of any one of SEQ ID NOs: 136-1215 or any variant thereof. In some embodiments, the composition comprises at least 90 gRNA polynucleotides of any one of SEQ ID NOs: 136-1215 or any variant thereof. In some embodiments, the composition comprises at least 100 gRNA polynucleotides of any one of SEQ ID NOs: 136-1215 or any variant thereof. In some embodiments, the compositions comprises at least 200 gRNA polynucleotides of any one of SEQ ID NOs: 136-1215 or any variant thereof. In some embodiments, the composition comprises at least 300 gRNA polynucleotides of any one of SEQ ID NOs: 136-1215 or any variant thereof. In some embodiments, the composition comprises at least 400 gRNA polynucleotides of any one of SEQ ID NOs: 136-1215 or any variant thereof.

In some embodiments, the composition comprises at least 500 gRNA polynucleotides of any one of SEQ ID NOs: 136-1215 or any variant thereof. In some embodiments, the compositions comprises at least 1000 gRNA polynucleotides selected from the group consisting of SEQ ID NOs: 136-1215 or any variant thereof.

In some embodiments, the composition comprises gRNA polynucleotides comprising the sequences of SEQ ID NOs: 136-1215 or any variant thereof. In some embodiments, the composition comprises gRNA polynucleotides comprising the sequences of SEQ ID NOS: 136-1215. In some embodiments, the composition comprises a gRNA polynucleotide comprising any one of SEQ ID NOs: 1216-1315. In some embodiments, the composition comprises gRNA polynucleotides comprising SEQ ID NOs: 1216-1315.

In some aspects, the present application discloses guide RNA polynucleotides. In some embodiments, a guide RNA polynucleotide is the guide RNA polynucleotide of any one of the compositions described herein. In some embodiments, the guide RNA polynucleotides comprise the sequence of any one of SEQ ID NOs: 136-1315, or a variant of any thereof.

### Plasmids, Vectors and Libraries Thereof

### Plasmids

In some aspects, the present application discloses a plasmid comprising any one of the gRNA polynucleotides disclosed herein. A “plasmid” as described herein, may refer to a circular piece of DNA comprising sequence elements for replication and expression of a polynucleotide (e.g., a gRNA). In some the replication element is an origin of replication (e.g., a bacterial origin of replication). In some embodiments, the expression sequence elements comprising a promoter and a terminator. In some embodiments, the plasmid comprises a selection marker (e.g., an antibiotic marker).

A “plasmid library” as described herein may comprise at least two plasmids, as described herein, encoding different gRNA polynucleotides. In some embodiments, the plasmid library comprises at least 3, (e.g., at least 4, at least 5, at least 6, at least 7, at least 8, at least 9, at least 10, at least 11, at least 12, at least 13, at least 14, at least 15, at least 16, at least 17, at least 18, at least 19 at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 1000, at least 5000, at least 10000, at least 15000, at least 20000, at least 30000, at least 40000, at least 50000, at least 60000, at least 70000, at least 80000, at least 90000, at least 100000, at least 1000000) plasmids encoding different gRNA polynucleotides (e.g., gRNA polynucleotides disclosed herein or variants thereof).

gRNA Vector

In some embodiments, the present application discloses a gRNA vector comprising: (1) a first gRNA polynucleotide comprising a homology region that is complementary to a marker protein (e.g., the TCR/CD3 complex (any one of SEQ ID NO: 1383-1389) of a T cell), and (2) a second gRNA polynucleotide.

In some embodiments, the first gRNA polynucleotide comprising a homology region of SEQ ID NO: 1316. In some embodiments, the first guide RNA polynucleotide, when transfected into an immune cell (e.g., a T cell) is used as a marker for CRISPR activity based on loss of TCR/CD3 expression on the immune cell. In some embodiments, the first gRNA polynucleotide is operably linked to a promoter. In some embodiments, the promoter is a constitutively active promoter. In some embodiments, the promoter is a tissue-specific promoter (e.g., an immune cell-specific promoter). In some embodiments, the promoter is an inducible promoter (e.g., a tet or lac promoter). In some embodiments, the promoter is selected from the group consisting of a CMV promoter, an EF1a promoter, a CAG promoter, a PGK promoter, a H1 promoter or a U6 promoter. In some embodiments, the promoter is a U6 promoter.

In some embodiments, the second gRNA polynucleotide of the gRNA vector comprises any one of the guide RNA polynucleotides described herein. In some embodiments, the second gRNA polynucleotide comprises a sequence that is complementary to an sense or antisense sequence of a gene encoded by any one of SEQ ID NOs: 1-135 or a variant thereof. In some embodiments, the second gRNA polynucleotide comprises a sequence of any one of SEQ ID NOs: 136-1315 or a variant thereof. In some embodiments, the second gRNA polynucleotide comprises a sequence of any one of SEQ ID NOs: 136-1315. In some embodiments, the second gRNA polynucleotide comprises a negative control gRNA sequence as described herein. In some embodiments, the second gRNA polynucleotide comprises a sequence of any one of SEQ ID NOs: 1216-1315 or a variant thereof. In some embodiments, the second gRNA polynucleotide is operably linked to a promoter. In some embodiments, the promoter is a constitutively active promoter. In some embodiments, the promoter is a tissue-specific promoter (e.g., an immune cell-specific promoter). In some embodiments, the promoter is an inducible promoter (e.g., a tet or lac promoter). In some embodiments, the promoter is selected from the group consisting of a CMV promoter, an EF1a promoter, a CAG promoter, a PGK promoter, H1 promoter, or a U6 promoter. In some embodiments, the U6 promoter is from a non-human species. In some embodiments, the U6 promoter is from a human U6 promoter. In some embodiments, the U6 promoter is from cow, mice, rat, pig, yeast, dog, cat, drosophila, or C. elegans. In some embodiments, the promoter is a H1 promoter. In some embodiments, the first gRNA polynucleotide is operably linked to a first promoter and the second gRNA polynucleotide is operably linked to a second promoter. In some embodiments, the first and second promoters are the same promoter (i.e., two different copies of the same promoter). In some embodiments, the first and second promoters are different promoters (i.e., comprise different sequences). In some embodiments, the first promoter is constitutive and the second promoter is inducible or tissue-specific. In some embodiments, both the first and second promoters are constitutive and tissue-specific. In some embodiments, the first gRNA polynucleotide and second gRNA polynucleotide are operably linked to a single promoter (e.g., are transcribed as a single polynucleotide, e.g., and are cleaved after transcription).

In some embodiments, the first gRNA polynucleotide and the second gRNA polynucleotide are in an inverted orientation in the guide RNA vector. In some embodiments, the first gRNA polynucleotide and the second gRNA polynucleotide are in a tandem orientation in the guide RNA vector. In some embodiments, the first gRNA polynucleotide and the second gRNA polynucleotide are encoded in the same CRISPR array. In some embodiments, the first gRNA polynucleotide and the second gRNA polynucleotide are single guide RNAs.

In some embodiments, the gRNA vector comprises a selectable marker. A “selectable marker” may refer to molecule that allows for detection of a biological event. For example, a selectable marker may provide evidence that a cell is expressing the gRNA vector. In some embodiments, the selectable marker is a guide RNA (e.g., the first guide RNA described above) that is capable of knocking out a gene (e.g. a cell surface gene), wherein the knockout of the gene can be used as a selectable marker. In some embodiments, the selectable marker is fluorescent marker. In some embodiments, the selectable marker provides resistance to a toxin (e.g., Geneticin, Hygromycin B, Puromycin or Zeocin). In some embodiments, the selectable maker is compatible for use in humans. In some embodiments, the selectable marker is expression of a non-endogenous cell surface protein. In some embodiments, the selectable marker is selected from the group consisting of LGNFR (Nerve Growth Factor Receptor), EGFR, CD19, CD20, CD34 and truncated versions thereof. In some embodiments, the selectable marker is LNGFR. In some embodiments, the selectable maker is operably linked to a promoter (e.g., a promoter described herein). In some embodiments, the selectable marker is operably linked to a weak promoter (e.g., PGK or UBC). In some embodiments, the selectable marker is operably linked to a tissue specific promoter (e.g., the HP1, CD14, CD43, CD45, C68, elastase, endoglin, fibronectin, Flt, GFAP, GPIIb, ICAM-2, mIFN-beta, Mb, NphsI, OG-2, SP-B, SYN1, or WASP gene promoter). In some embodiments, the selectable marker selected for CRISPR activity. In some embodiments, the selectable marker is based on negative selection (e.g., the knockout of a target protein). In some embodiments, the selectable marker is knockout of CD3.

In some embodiments, the gRNA vector comprises a suicide gene. A “suicide gene” refers to a gene, that when transcribed or translated, is toxic to the host cell (e.g., the cell transcribing or translating the suicide gene). In some embodiments, the suicide gene is operably linked to a promoter (e.g., a promoter described herein). In some embodiments, the suicide gene is operably linked to an inducible promoter. In some embodiments, the suicide gene is operably linked to a weak promoter (e.g., a PGK promoter). In some embodiments, the suicide gene is selected from the group consisting of icaspase9, tEGFR, tCD29, CD20, and tHer2. In some embodiments, the suicide gene is icaspase9.

In some embodiments, the gRNA vector is a viral vector as described above. In some embodiments, the gRNA vector is selected from the group consisting of an adeno-associated vector, a retroviral vector, or a lentiviral vector. In some embodiments, the lentiviral vector is a third-generation self-inactivating (SIN) lentiviral vector.

In some embodiments, the gRNA vector comprises a sequence of SEQ ID NO: 1318 and a sequence of a guide RNA polynucleotide as described herein. In some embodiments, the gRNA vector comprises a sequence of SEQ ID NO: 1318 and a sequence of a guide RNA polynucleotide homology region of any one of SEQ ID NOs: 136-1215 or a variant thereof. In some embodiments, the gRNA vector comprises a sequence of SEQ ID NO: 1318 and a sequence of a guide RNA polynucleotide homology region of any one of SEQ ID NOs: 136-1315 or a variant thereof. In some embodiments, the gRNA vector further comprises a polynucleotide encoding a CRISPR protein (e.g., Cas9 or Cas12) as described above.

gRNA Vector Library

A “gRNA vector library” refers to a composition comprising at least 2 gRNA vectors encoding different gRNA polynucleotides as described herein. In some embodiments, the gRNA vector library comprises at least 3 gRNA vectors encoding different gRNA polynucleotides. In some embodiments, the gRNA vector library comprises at least 4 gRNA vectors encoding different gRNA polynucleotides. In some embodiments, the gRNA vector library comprises at least 5 gRNA vectors encoding different gRNA polynucleotides. In some embodiments, the gRNA vector library comprises at least 6 gRNA vectors encoding different gRNA polynucleotides. In some embodiments, the gRNA vector library comprises at least 7 gRNA vectors encoding different gRNA polynucleotides. In some embodiments, the gRNA vector library comprises at least 8 gRNA vectors encoding different gRNA polynucleotides. In some embodiments, the gRNA vector library comprises at least 9 gRNA vectors encoding different gRNA polynucleotides. In some embodiments, the gRNA vector library comprises at least 10 gRNA vectors encoding different gRNA polynucleotides. In some embodiments, the gRNA vector library comprises at least 11 gRNA vectors encoding different gRNA polynucleotides. In some embodiments, the gRNA vector library comprises at least 12 gRNA vectors encoding different gRNA polynucleotides. In some embodiments, the gRNA vector library comprises at least 13 gRNA vectors encoding different gRNA polynucleotides. In some embodiments, the gRNA vector library comprises at least 14 gRNA vectors encoding different gRNA polynucleotides. In some embodiments, the gRNA vector library comprises at least 15 gRNA vectors encoding different gRNA polynucleotides. In some embodiments, the gRNA vector library comprises at least 16 gRNA vectors encoding different gRNA polynucleotides. In some embodiments, the gRNA vector library comprises at least 17 gRNA vectors encoding different gRNA polynucleotides. In some embodiments, the gRNA vector library comprises at least 18 gRNA vectors encoding different gRNA polynucleotides. In some embodiments, the gRNA vector library comprises at least 19 gRNA vectors encoding different gRNA polynucleotides. In some embodiments, the gRNA vector library comprises at least 20 gRNA vectors encoding different gRNA polynucleotides. In some embodiments, the gRNA vector library comprises at least 21 gRNA vectors encoding different gRNA polynucleotides. In some embodiments, the gRNA vector library comprises at least 22 gRNA vectors encoding different gRNA polynucleotides. In some embodiments, the gRNA vector library comprises at least 23 gRNA vectors encoding different gRNA polynucleotides. In some embodiments, the gRNA vector library comprises at least 24 gRNA vectors encoding different gRNA polynucleotides. In some embodiments, the gRNA vector library comprises at least 25 gRNA vectors encoding different gRNA polynucleotides. In some embodiments, the gRNA vector library comprises at least 26 gRNA vectors encoding different gRNA polynucleotides. In some embodiments, the gRNA vector library comprises at least 27 gRNA vectors encoding different gRNA polynucleotides. In some embodiments, the gRNA vector library comprises at least 28 gRNA vectors encoding different gRNA polynucleotides. In some embodiments, the gRNA vector library comprises at least 29 gRNA vectors encoding different gRNA polynucleotides. In some embodiments, the gRNA vector library comprises at least 30 gRNA vectors encoding different gRNA polynucleotides. In some embodiments, the gRNA vector library comprises at least 40 gRNA vectors encoding different gRNA polynucleotides. In some embodiments, the gRNA vector library comprises at least 50 gRNA vectors encoding different gRNA polynucleotides. In some embodiments, the gRNA vector library comprises at least 60 gRNA vectors encoding different gRNA polynucleotides. In some embodiments, the gRNA vector library comprises at least 70 gRNA vectors encoding different gRNA polynucleotides. In some embodiments, the gRNA vector library comprises at least 80 gRNA vectors encoding different gRNA polynucleotides. In some embodiments, the gRNA vector library comprises at least 90 gRNA vectors encoding different gRNA polynucleotides. In some embodiments, the gRNA vector library comprises at least 100 gRNA vectors encoding different gRNA polynucleotides. In some embodiments, the gRNA vector library comprises at least 200 gRNA vectors encoding different gRNA polynucleotides. In some embodiments, the gRNA vector library comprises at least 300 gRNA vectors encoding different gRNA polynucleotides. In some embodiments, the gRNA vector library comprises at least 400 gRNA vectors encoding different gRNA polynucleotides. In some embodiments, the gRNA vector library comprises at least 500 gRNA vectors encoding different gRNA polynucleotides. In some embodiments, the gRNA vector library comprises at least 1000 gRNA vectors encoding different gRNA polynucleotides. In some embodiments, the gRNA vector library comprises at least 5000 gRNA vectors encoding different gRNA polynucleotides. In some embodiments, the gRNA vector library comprises at least 10000 gRNA vectors encoding different gRNA polynucleotides. In some embodiments, the gRNA vector library comprises at least 15000 gRNA vectors encoding different gRNA polynucleotides. In some embodiments, the gRNA vector library comprises at least 20000 gRNA vectors encoding different gRNA polynucleotides. In some embodiments, the gRNA vector library comprises at least 30000 gRNA vectors encoding different gRNA polynucleotides. In some embodiments, the gRNA vector library comprises at least 40000 gRNA vectors encoding different gRNA polynucleotides. In some embodiments, the gRNA vector library comprises at least 50000 gRNA vectors encoding different gRNA polynucleotides. In some embodiments, the gRNA vector library comprises at least 60000 gRNA vectors encoding different gRNA polynucleotides. In some embodiments, the gRNA vector library comprises at least 70000 gRNA vectors encoding different gRNA polynucleotides. In some embodiments, the gRNA vector library comprises at least 80000 gRNA vectors encoding different gRNA polynucleotides. In some embodiments, the gRNA vector library comprises at least 90000 gRNA vectors encoding different gRNA polynucleotides. In some embodiments, the gRNA vector library comprises at least 100000 gRNA vectors encoding different gRNA polynucleotides. In some embodiments, the gRNA vector library comprises at least 1000000 gRNA vectors encoding different gRNA polynucleotides.

In some embodiments, the gRNA vector library comprises at least 2 gRNA each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 3 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 4 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 5 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 6 gRNA each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 7 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 8 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 9 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 10 gRNA vectors each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 11 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 12 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 13 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 14 gRNA vectors each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 15 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 16 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 17 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 18 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 19 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 20 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 21 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 22 gRNA vectors each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 23 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 24 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 25 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 26 gRNA vectors each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 27 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 28 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 29 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 30 gRNA vectors each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 40 gRNA vectors each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 50 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 60 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 70 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 80 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 90 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 100 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 200 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 300 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 400 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 500 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 1000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 5000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 10000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof.

In some embodiments, the gRNA vector library comprises at least 15000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 20000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 30000 gRNA vectors encoding different gRNA polynucleotides each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 40000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 50000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 60000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 70000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 80000 gRNA vectors encoding different gRNA polynucleotides each comprising a different gRNA polynucleotide homology region that is complementary to a sense strand or an antisense strand of a different gene encoded by any one of SEQ ID NOS: 1-135 or a variant thereof. In some embodiments, the gRNA vector library comprises at least 90000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 100000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof. In some embodiments, the gRNA vector library comprises at least 1000000 gRNA vectors encoding different gRNA polynucleotides each comprising a different gRNA polynucleotide homology region that is complementary to a sense strand or an antisense strand of a different gene encoded by any one of SEQ ID NOs: 1-135 or a variant thereof.

In some embodiments, the gRNA vector library comprises 135 gRNA vectors each of the gRNA vectors comprising a first guide RNA polynucleotide that targets a marker protein (e.g., CD3) and a second guide RNA comprising a homology region that is complementary to a sense strand or antisense strand of a gene encoded by any one of SEQ ID NOs: 1-135 or a variant thereof. In some embodiments, the gRNA vector library comprises 1080 gRNA vectors each of the gRNA vectors comprising a first guide RNA polynucleotide that targets a marker protein (e.g., CD3) and a second guide RNA comprising a homology region that is complementary to a sense strand or antisense strand of a gene encoded by any one of SEQ ID NOs: 1-135 or a variant thereof.

In some embodiments, the gRNA vector library comprises gRNA vectors comprising gRNA polynucleotide homolog regions that are complementary to at least 2 (e.g., at least 3, at least 4, at least 5, at least 6, at least 7, at least 8, at least 9, at least 10, at least 11, at least 12, at least 13, at least 14, at least 15, at least 16, at least 17, at least 18, at least 19 at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 120 or at least 135) gene sequences of any one of SEQ ID NOs: 1-135.

In some embodiments, the gRNA vector library comprises any one of the guide RNA polynucleotides of any of the compositions described herein. In some embodiments, the gRNA vector library comprises guide RNA polynucleotides comprising any one of SEQ ID NOs: 136-1215 or a variant thereof. In some embodiments, the gRNA vector library comprises guide RNA polynucleotides comprising any one of SEQ ID NOs: 136-1315 or a variant thereof.

### CAR Vector

In some embodiments, the present application provides vectors comprising a sequence encoding a chimeric antigen receptor (CAR).

### Chimeric Antigen Receptors (CARs)

The terms “chimeric antigen receptor” or “CAR” or “CARs”, as used herein, refer to engineered T cell receptors, which graft a ligand or antigen specificity onto T cells (for example, naive T cells, central memory T cells, effector memory T cells or combinations thereof). CARs are also known as artificial T cell receptors, chimeric T cell receptors or chimeric immunoreceptors.

A CAR places a chimeric antigen binding domain that specifically binds a target, e.g., a polypeptide, expressed on the surface of a cell to be targeted for a T cell response onto a construct including a transmembrane domain and intracellular domain(s) of a T cell receptor molecule. In some embodiments, the chimeric antigen binding domain includes the antigen domain(s) of an antibody reagent that specifically binds an antigen expressed on a cell to be targeted for a T cell response. In some embodiments, the chimeric antigen binding domain includes a ligand that specifically binds an antigen expressed on a cell to be targeted for a T cell response.

As used herein, a “CAR-T cell” or “CAR-T” refers to a T cell that expresses a CAR. When expressed in a T cell, CARs have the ability to redirect T cell specificity and reactivity toward a selected target in a non-MHC-restricted manner, exploiting the antigen binding properties of monoclonal antibodies. The non-MHC-restricted antigen recognition gives T cells expressing CARs the ability to recognize an antigen independent of antigen processing, thus bypassing a major mechanism of tumor escape.

As used herein, a “CAR-NK cell” or “CAR-NK” refers to a natural killer (NK) cell that expresses a CAR.

In some embodiments, the CAR excludes a CD8 signal peptide as described herein. As can be determined by those of skill in the art, various functionally similar or equivalent components of these CARs can be swapped or substituted with one another, as well as other similar or functionally equivalent components known in the art or listed herein.

Any cell-surface moiety can be targeted by a CAR. Often, the target will be a cell-surface polypeptide that may be differentially or preferentially expressed on a cell that one wishes to target for a T cell response. In some embodiments, the extracellular target binding domain binds to any one of CD19, CD37, CD70, CD79b, TACI, BCMA, MUC1, MUC16, B7H3, mesothelin, CD70, PSMA, PSCA, EGFRVIII, claudin6, binds to any pair of CD19/CD79b, BCMA/TACI, or is a TriPRIL antigen binding domain, e.g., as described in PCT/US2020/065733, PCT/US2020/036108, PCT/US2018/013215, PCT/US2018/013213, PCT/US2018/027783, PCT/US2018/013221, PCT/US2018/022974, PCT/US2019/042268, PCT/US2019/038518, PCT/US2019/066357, PCT/US2019/013103, PCT/US2019/017727, PCT/US2020/051018, and/or PCT/US2018/013095.

### Antigen Binding Domain

As used herein, the term “antigen binding domain” refers to a polypeptide found on the outside of the cell that is sufficient to facilitate binding to a target. The extracellular target binding domain will specifically bind to its binding partner, i.e., the target. As non-limiting examples, the antigen binding domain can include an antigen domain of an antibody or antibody reagent, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway. In some embodiments, the ligand can be non-native to the genome. In some embodiments, the ligand has a conserved function across at least two species.

Any cell-surface moiety can be targeted by a CAR. In some embodiments, the target will be a cell-surface polypeptide that may be differentially or preferentially expressed on a cell that one wishes to target for a T cell response. To target Tregs, antibody reagents can be targeted against, e.g., Glycoprotein A Repetitions Predominant (GARP), latency-associated peptide (LAP), CD25, CTLA-4, ICOS, TNFR2, GITR, OX40, 4-1BB, and LAG-3.

In some embodiments, the CAR vector comprises a CAR polynucleotide encoding an antigen binding domain that binds to any one of CD19, CD79b, TACI, BCMA, MUC1, MUC16, B7H3, mesothelin, CD70, PSMA, PSCA, EGFRVIII, claudin6, binds to any pair of CD19/CD79b, BCMA/TACI, or is a TriPRIL antigen binding domain.

In some embodiments, the CAR vector comprises a CAR comprising an antigen binding domain that binds mesothelin. In some embodiments, the mesothelin CAR comprises a polynucleotide encoding an extracellular binding domain comprising a mesothelin antibody (e.g., scFv). In some embodiments, the mesothelin scFv comprises a VH domain of SEQ ID NO: 1366 and a VL domain of SEQ ID NO: 1367, or a variant thereof. In some embodiments, the mesothelin scFv comprises SEQ ID NO: 1368 or SEQ ID NO: 1369, or a variant thereof.

TABLE 1
Mesothelin antibody sequences

| SEQ ID | | |
|---|---|---|
| NO: | Name | Sequence |
| 1366 | MGHmeso 1 | MECNWILPFILSVTSGVYSEILL |
| | antibody VH | QQTGTVLARPGTSVKMSCKASGY |
| | | TFTNYRMHWVKQRPGQGLEWIGG |
| | | IYPGNSDTNYNQKFKDKAKLTAV |
| | | TSTSTANMELSSLTNEDSAVYYC |
| | | LRGIRGSYFDYWGQGTTLTVSS |
| 1367 | MGHmeso 1 | METDTILLWVLLLWVPGSTGDI |
| | antibody VL | VLTQSPASLAVSLGQRATISCK |
| | | ASQSVDYDGDSYMNWYQQKPGQ |
| | | PPKLLIYAASNLESGIPARFSG |
| | | SGSGTDFTLNIHPVEEEDAATY |
| | | YCQQSNEDPSTFGGGTKLEVK |
| 1368 | MGHmeso 1 | MECNWILPFILSVTSGVYSEIL |
| | scFv (VL- | LQQTGTVLARPGTSVKMSCKAS |
| | VH) | GYTFTNYRMHWVKQRPGQGLEW |
| | | IGGIYPGNSDTNYNQKFKDKAK |
| | | LTAVTSTSTANMELSSLTNEDS |
| | | AVYYCLRGIRGSYFDYWGQGTT |
| | | LTVSSGGGGSGGGGSGGGGSGG |
| | | GGSMETDTILLWVLLLWVPGST |
| | | GDIVLTQSPASLAVSLGQRATI |
| | | SCKASQSVDYDGDSYMNWYQQK |
| | | PGQPPKLLIYAASNLESGIPAR |
| | | FSGSGSGTDFTLNIHPVEEEDA |
| | | ATYYCQQSNEDPSTFGGGTKLE |
| | | VK |
| 1369 | MGHmeso 1 | METDTILLWVLLLWVPGSTGDI |
| | scFv (VH- | VLTQSPASLAVSLGQRATISCK |
| | VL) | ASQSVDYDGDSYMNWYQQKPGQP |
| | | PKLLIYAASNLESGIPARFSGS |
| | | GSGTDFTLNIHPVEEEDAATYY |
| | | CQQSNEDPSTFGGGTKLEVKGG |
| | | GGSGGGGSGGGGSGGGGSMECN |
| | | WILPFILSVTSGVYSEILLQQT |
| | | GTVLARPGTSVKMSCKASGYTF |
| | | TNYRMHWVKQRPGQGLEWIGGI |
| | | YPGNSDTNYNQKFKDKAKLTAV |
| | | TSTSTANMELSSLTNEDSAVYY |
| | | CLRGIRGSYFDYWGQGTTLTVS |
| | | S |

### Hinge and Transmembrane Domains

In some embodiments, the CAR polypeptide further comprises a transmembrane domain, e.g., a hinge/transmembrane domain, which joins the antigen binding domain to the intracellular signaling domain. The binding domain of the CAR is, in some embodiments, followed by one or more “hinge domains,” which plays a role in positioning the antigen binding domain away from the effector cell surface to enable proper cell/cell contact, antigen binding and activation. A CAR may include one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8alpha), CD4, CD28, 4-1BB, and CD7, which may be wild-type hinge regions from these molecules or may be altered. In some embodiments, the CAR comprises polynucleotide encoding CD8alpha hinge/transmembrane domain. In some embodiments, the CAR comprises a polynucleotide encoding a 41BB intracellular domain.

In some embodiments, the hinge region is derived from the hinge region of an immunoglobulin like protein (e.g., lgA, lgD, lgE, lgG, or lgM), CD28, or CD8. In some embodiments, the hinge domain includes a CD8a hinge region.

As used herein, “transmembrane domain” (TM domain) refers to the portion of the CAR that fuses the extracellular binding portion, in some embodiments via a hinge domain, to the intracellular portion (e.g., the costimulatory domain and intracellular signaling domain) and anchors the CAR to the plasma membrane of the immune effector cell. The transmembrane domain is a generally hydrophobic region of the CAR, which crosses the plasma membrane of a cell. The TM domain can be the transmembrane region or fragment thereof of a transmembrane protein (for example a Type I transmembrane protein or other transmembrane protein), an artificial hydrophobic sequence, or a combination thereof. While specific examples are provided herein and used herein, other transmembrane domains will be apparent to those of skill in the art and can be used in connection with alternate embodiments of the technology. A selected transmembrane region or fragment thereof would preferably not interfere with the intended function of the CAR.

As used in relation to a transmembrane domain of a protein or polypeptide, “fragment thereof” refers to a portion of a transmembrane domain that is sufficient to anchor or attach a protein to a cell surface.

In some embodiments, the transmembrane domain or fragment thereof of the CAR described herein includes a transmembrane domain selected from the transmembrane domain of an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c,

ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

As used herein, a “hinge/transmembrane domain” refers to a domain including both a hinge domain and a transmembrane domain. For example, a hinge/transmembrane domain can be derived from the hinge/transmembrane domain of CD8, CD28, CD7, or 4-1BB. In some embodiments, the hinge/transmembrane domain of a CAR or fragment thereof is derived from or includes the hinge/transmembrane domain of CD8 (e.g., any one of SEQ ID NOs: 1, or variants thereof). CD8 is an antigen preferentially found on the cell surface of cytotoxic T lymphocytes. CD8 mediates cell-cell interactions within the immune system, and acts as a T cell co-receptor. CD8 consists of an alpha (CD8alpha or CD8a) and beta (CD813 or CD8b) chain. CD8a sequences are known for a number of species, e.g., human CD8a, (NCBI Gene ID: 925) polypeptide (e.g., NCBI Ref Seq NP 001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like.

Homologs and/or orthologs of human CD8 are readily identified for such species by one of skill in the art, e.g., using the NCBI ortholog search function or searching available sequence data for a given species for sequence similar to a reference CD8 sequence.

In some embodiments, the CD8 hinge and transmembrane sequence corresponds to the amino acid sequence of SEQ ID NO: 1371 (TTTPAPRPPTPAPTIASQPLSLRPEACRPAAGGA VHTRGLDFACDIYIWAPLAGTCGVLLLSLVITLYC); or includes the sequence of SEQ ID NO: 1; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 1371.

### Co-Stimulatory Domains

Each CAR described herein optionally includes the intracellular domain of one or more co-stimulatory molecule or co-stimulatory domain. As used herein, the term “co-stimulatory domain” refers to an intracellular signaling domain of a co-stimulatory molecule. Co-stimulatory molecules are cell surface molecules other than antigen receptors or Fe receptors that provide a second signal required for efficient activation and function of T lymphocytes upon binding to antigen. The co-stimulatory domain can be, for example, the co-stimulatory domain of 4-1BB, CD27, CD28, or OX40. In one example, a 4-1BB intracellular domain (ICD) can be used (see, e.g., below and SEQ ID NOs: 1372 (KRGRKKLLYIFKQPFMRPVQTTQEEDGCSCRFPEE EEGGCEL), or variants thereof). Additional illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In some embodiments, the intracellular domain is the intracellular domain of 4-1 BB. 4-1 BB (CD137; TNFRS9) is an activation induced costimulatory molecule, and is an important regulator of immune responses.

4-1BB is a membrane receptor protein, also known as CD137, which is a member of the tumor necrosis factor (TNF) receptor superfamily. 4-1BB is expressed on activated T lymphocytes. 4-1BB sequences are known for a number of species, e.g., human 4-1 BB, also known as TNFRSF9 (NCBI Gene 25 ID: 3604) and mRNA (NCBI Reference Sequence: NM_001561.5). 4-1BB can refer to human 4-1BB, including naturally occurring variants, molecules, and alleles thereof. In some embodiments of any of the aspects, e.g., in veterinary applications, 4-1BB can refer to the 4-1BB of, e.g., dog, cat, cow, horse, pig, and the like. Homologs and/or orthologs of human 4-1BB are readily identified for such species by one of skill in the art, e.g., using the NCBI ortholog search function or searching available sequence data for a given species for sequence similar to a reference 4-1 BB sequence.

### Intracellular Signaling Domains

In some embodiments, the CAR comprises a polynucleotide encoding a CD3zeta intracellular signaling domain.

The properties of the intracellular signaling domain(s) of the CAR can vary as known in the art and as disclosed herein, but the chimeric target/antigen binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3-zeta signals upon antigen binding. So-called “second-generation” CARs include those that provide both co-stimulation (e.g., CD28 or CD137) and activation (CD3-zeta;) domains, and so-called “third-generation” CARs include those that provide multiple costimulatory (e.g., CD28 and CD137) domains and activation domains (e.g., CD3-zeta). In various embodiments, the CAR is selected to have high affinity or avidity for the target/antigen—for example, antibody-derived target or antigen binding domains will generally have higher affinity and/or avidity for the target antigen than would a naturally occurring T cell receptor. This property, combined with the high specificity one can select for an antibody provides highly specific T cell targeting by CAR-T cells.

CARs as described herein include an intracellular signaling domain. An “intracellular signaling domain” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain. In various examples, the intracellular signaling domain is from CD3-zeta; (see, e.g., below). Additional non-limiting examples of immunoreceptor tyrosine-based activation motif (ITAM)-containing intracellular signaling domains that are of particular use in the technology include those derived from TCR-zeta, FcR-gamma, FcR-beta, CD3-gamma, CD3-theta, CD3-sigma, CD3-eta, CD3-epsilon, CD3-zeta, CD22, CD79a, CD79b, and CD66d.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3-gamma chain, a CD3delta chain, and two CD3-epsilon chains.

These chains associate with a molecule known as the T cell receptor (TCR) and the CD3-zeta to generate an activation signal in T lymphocytes. A complete TCR complex includes a TCR, CD3-zeta, and the complete CD3 complex.

In some embodiments of any aspect, a CAR polypeptide described herein includes an intracellular signaling domain that includes an Immunoreceptor Tyrosine-based Activation Motif or ITAM from CD3-zeta, including variants of CD3-zeta such as ITAM-mutated CD3-zeta, CD3-eta, or CD3-theta. In some embodiments of any aspect, the ITAM includes three motifs of ITAM of CD3-zeta (ITAM3). In some embodiments of any aspect, the three motifs of ITAM of CD3-zeta are not mutated and, therefore, include native or wild-type sequences. In some embodiments, the CD3-zeta sequence includes the sequence of a CD3-zeta as set forth in the sequences provided herein, e.g., a CD3-zeta sequence of SEQ ID NO: 1373 (RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQA LPPR), or variants thereof.

For example, a CAR polypeptide described herein includes the intracellular signaling domain of CD3-zeta. In some embodiments, the CD3-zeta intracellular signaling domain corresponds to an amino acid sequence of SEQ ID NO: 1373 or includes a sequence of SEQ ID NOs: 1373; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to a sequence of SEQ ID NOs: 1373.

In some embodiments, the intracellular domain is the intracellular domain of a 4-1 BB. In some embodiments, the 4-1BB intracellular domain corresponds to an amino acid sequence selected from SEQ ID NO: 2; or includes a sequence selected from SEQ ID NO: 2; or includes at least 75%, at least 80%, at least 85%, 35 at least 90%, at least 95%, at least 96%, at least 97%, at least 98%, at least 99%, or at least 100% sequence identity to a sequence selected from SEQ ID NO: 2.

Individual CAR and other construct components as described herein can be used with one another and swapped in and out of various constructs described herein, as can be determined by those of skill in the art. Each of these components can include or consist of any of the corresponding sequences set forth herein, or variants thereof.

A more detailed description of CARs and CAR-T cells can be found in Maus et al., Blood 123:2624-2635, 2014; Reardon et al., Neuro-Oncology 16:1441-1458, 2014; Hoyos et al., Haematologica 97:1622, 2012; Byrd et al., J. Clin. Oncol. 32:3039-3047, 2014; Maher et al., Cancer Res 69:4559-4562, 2009; and Tamada et al., Clin. Cancer Res. 18:6436-6445, 2012; each of which is incorporated by reference herein in its entirety.

### Signal Peptide

In some embodiments, a CAR polypeptide as described herein includes a signal peptide. Signal peptides can be derived from any protein that has an extracellular domain or is secreted. A CAR polypeptide as described herein may include any signal peptides known in the art. In some embodiments, the CAR polypeptide includes a CD8 signal peptide, e.g., a CD8 signal peptide corresponding to the amino acid sequence of SEQ ID NO: 1374 (MALPVTALLLPLALLLHAARP), or including the amino acid sequence of SEQ ID NO: 5, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 1374.

In further embodiments, a CAR polypeptide described herein may optionally exclude one of the signal peptides described herein, e.g., a CD8 signal peptide of SEQ ID NO: 1374 or an lgK signal peptide of SEQ ID NO: 1375 (METDTLLLWVLLLWVPGSTGD).

### Linker Domain

In some embodiments, the CAR further includes a linker domain. As used herein, “linker domain” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another. Linker sequences useful for the invention can be from 2 to 100 amino acids, 5 to 50 amino acids, 10 to 15 amino acids, 15 to 20 amino acids, or 18 to 20 amino acids in length, and include any suitable linkers known in the art. For instance, linker sequences useful for the invention include, but are not limited to, glycine/serine linkers, e.g., GGGSGGGSGGGS (SEQ ID NO: 1376) and Gly4Ser (G4S) (SEQ ID NO: 1378) linkers such as (G4S) 3 (GGGGSGGGGSGGGGS (SEQ ID NO: 1377)) and (G4S) 4 (GGGGSGGGGSGGGGSGGGGS (SEQ ID NO: 1378)); the linker sequence of GSTSGSGKPGSGEGSTKG (SEQ ID NO: 1379) as described by Whitlow et al., Protein Eng. 6 (8): 989-95, 1993, the contents of which are incorporated herein by reference in its entirety; the linker sequence of GGSSRSSSSGGGGSGGGG (SEQ ID NO: 1380) as described by Andris-Widhopf et al., Cold Spring Harb. Protoc. 2011 (9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18 (1): 75-80, 2000, the contents of which are incorporated herein by reference in its entirety. Longer linkers may be used when it is desirable to ensure that two adjacent domains do not sterically interfere with one another.

Furthermore, linkers may be cleavable or non-cleavable. Examples of cleavable linkers include 2A linkers (e.g., P2A (SEQ ID NO: 1381, GSGATNFSLLKQAGDVEENPGP) and T2A (SGGGGEGRGSLLTCGDVEENPGPR, SEQ ID NO: 1382), 2A-like linkers or functional equivalents thereof and combinations thereof.

For example, a P2A linker sequence can correspond to the amino acid sequence of SEQ ID NO: 1381. In various examples, linkers having sequences as set forth herein, or variants thereof, are used. It is to be understood that the indication of a particular linker in a construct in a particular location does not mean that only that linker can be used there. Rather, different linker sequences (e.g., P2A and T2A) can be swapped with one another (e.g., in the context of the constructs of the present invention), as can be determined by those of skill in the art. In some embodiments, the linker region is T2A derived from Thosea asigna virus. Non-limiting examples of linkers that can be used in this technology include T2A, P2A, E2A, BmCPV2A, and BmlFV2A. Linkers such as these can be used in the context of polyproteins, such as those described below. For example, they can be used to separate a CAR component of a polyprotein from a therapeutic agent (e.g., an antibody, such as a scFv, single domain antibody (e.g., a camelid antibody), or a bispecific antibody (e.g., a TEAM)) component of a polyprotein (see below).

### Full CARS

In some embodiments, the CAR is selected from a group consisting of (1) a CAR that binds to any one of CD19, CD79b, TACI, BCMA, MUC1, MUC16, B7H3, mesothelin, CD70, PSMA, PSCA, EGFRvIII, claudin6, (2) a CAR that binds to any pair of CD19/CD79b, BCMA/TACI, or (3) is a TriPRIL antigen binding domain. In some embodiments, the CAR polypeptide comprises an amino acid sequence with at least 75%, at least 80%, at least 85%, at least 90%, at least 91%, at least 92%, at least 93%, at least 94%, at least 95%, at least 96%, at least 97%, at least 98%, at least 99% or greater sequence identity of a sequence of any one of SEQ ID NOs: 1325-1365. In some embodiments, the CAR comprises an amino acid sequence of any one of SEQ ID NOs: 1325-1365. In some embodiments, the CAR polypeptide consists of an amino acid sequence of any one of SEQ ID NOs: 1325-1365. In some embodiments, the CAR polypeptide comprises an amino acid sequence of any one of SEQ ID NOs: 1325-1365.

In some embodiments, the CAR comprises a polynucleotide encoding a Mesothelin scFv, a CD8alpha hinge/transmembrane, a 41BB intracellular domain, and a CD3zeta signaling domains. In some embodiments, the CAR polypeptide comprises an amino acid sequence with at least 75%, at least 80%, at least 85%, at least 90%, at least 91%, at least 92%, at least 93%, at least 94%, at least 95%, at least 96%, at least 97%, at least 98%, at least 99% or greater sequence identity to a sequence of any one of SEQ ID NOs: 1325-1328. In some embodiments, the CAR comprises an amino acid sequence of any one of SEQ ID NOS: 1325-1328. In some embodiments, the CAR polypeptide comprises an amino acid sequence of any one of SEQ ID NOs: 1325-1328.

### CAR Vectors

In some embodiments, the CAR vector is a viral vector as described above. In some embodiments, the CAR vector is an adeno-associated vector, a retro-viral vector, or a lentiviral vector. In some embodiments, the CAR vector is a self-inactivating vector (a self-inactivating lentiviral vector or a self-inactivating retro-viral vector). In some embodiments, the CAR vector is a third-generation self-inactivating (SIN) lentiviral vector. In some embodiments, the CAR is operably linked to a promoter. In some embodiments, the promoter is a constitutively active promoter. In some embodiments, the promoter is a chemically inducible promoter. In some embodiments, the promoter is a weak constitutively active promoter. In some embodiments, the promoter is a promoter described herein. In some embodiments, the promoter is a EF1a or EF1a-short promoter.

In some embodiments, the CAR vector comprises a suicide gene. A “suicide gene” may refer to a gene, that upon transcription or translation, will result in cell death. Thus, suicide genes may be included on the CAR vector to deactivate CAR therapy after treatment is complete or if the subject has adverse side effects. In some embodiments, the suicide gene is selected from the group consisting of icaspase9, tEGFR, tCD29, CD20, and tHer2. In some embodiments, the CAR vector comprises a selectable marker (e.g., a reporter). In some embodiments, the selectable marker is selected from the group consisting of truncated CD34, tEGFR, tCD19, tCD20, tCD34, and tHer2. In some embodiments, the selected marker is CD34 or LNGFR.

In some embodiments, the CAR vector comprises a 2A ribosomal skip element or an internal ribosome entry site (IRES) between the CAR polynucleotide sequence and the selectable marker sequence.

In some embodiments, the CAR vector further comprises a nucleotide sequence encoding a CRISPR protein as described herein. In some embodiments, the CAR vector does not comprise a nucleotide sequence encoding a CRISPR protein.

In some embodiments, the CAR vector comprises a CAR selected from the groups consisting of (1) a CAR that binds to any one of CD19, CD79b, TACI, BCMA, MUC1, MUC16, B7H3, mesothelin, CD70, PSMA, PSCA, EGFRVIII, claudin6, (2) a CAR that binds to any pair of CD19/CD79b, BCMA/TACI, or (3) is a TriPRIL antigen binding domain.

In some embodiments, the CAR vector comprises a polynucleotide sequence that is at least 85%, at least 90%, at least 91%, at least 92%, at least 93%, at least 94%, at least 95%, at least 96%, at least 97%, at least 98%, at least 99%, or at least 100% identical to SEQ ID NO: 1319. In some embodiments, the CAR vector comprises a polynucleotide sequence of SEQ ID NO: 1319.

### CAR-T Cells, NK-Cells and Mutant Libraries Cell Libraries

In some aspects the disclosure is directed to an immune cell (e.g., a CAR-T cell or a CAR-NK cell) comprising a guide RNA vector described herein and/or comprising a mutation in a gene corresponding to the gene sequence of any one of SEQ ID NOs: 1-135. The disclosure is directed, in part, to the idea of introducing one or more mutations into an immune cell by contacting the immune cell with a guide RNA vector and providing a CRISPR protein (e.g., as part of the guide RNA vector or encoded on a separate vector). Expression of the guide RNA vector and a CRISPR protein can result in a mutation in the immune cell in or proximal to a sequence complementary to a homology region of a gRNA polynucleotide encoded by the gRNA vector. Accordingly, the disclosure is directed to immune cells comprising the guide RNA vector, immune cells comprising a mutation produced by action of the one or more gRNA polynucleotides encoded by the guide RNA vector and a CRISPR protein, and immune cells comprising both thereof. In some embodiments, the immune cell comprises (i) a guide RNA vector and/or a mutation in a gene corresponding to the gene sequence of any one of SEQ ID NOs: 1-135; and (ii) a CAR vector as described herein. In some embodiments, the immune cell comprises one, two, or three of: a mutation in a gene corresponding to the gene sequence of any one of SEQ ID NOs: 1-135 or a variant thereof, a gRNA polynucleotide as described herein, a plasmid as described herein, or a gRNA vector as described herein. In some embodiments, the mammalian cell further comprises a CAR vector as described herein. In some embodiments, the mammalian cell further comprises an mRNA encoding a CAR and/or a polypeptide encoding a CAR.

In some embodiments, the immune cell comprises a mutation in a gene corresponding to the gene sequence of any one of SEQ ID NOs: 1-135 or a variant thereof. In some embodiments, the mutation is a result of CRISPR cleavage of a gene. In some embodiments, the mutation is a loss of function mutation. In some embodiments, the mutation in a single nucleotide polymorphism. In some embodiments, the mutation is a transversion. In some embodiments, the mutation is a transition. In some embodiments, the mutation changes the protein sequence of the target gene. In some embodiments, the mutations introduces an early stop codon. In some embodiments, the mutation is an insertion or deletion. In some embodiments, the immune cell comprises a gRNA polynucleotide as described herein. In some embodiments, the immune cell comprises a gRNA vector as described herein. In some embodiments, the immune cell further comprises a CAR vector as described herein. In some embodiments, the immune cell comprises a CAR as described herein.

In some embodiments, the immune cell comprising a gRNA vector and a CAR vector further comprises selected markers (e.g., reporters as described herein). In some embodiments, the immune cell is CD34 positive and CD3 negative. In some embodiments, the immune cell is CD34 positive, CD3 negative and LNGFR positive.

The immune cell or tissue can be of human, primate, hamster, rabbit, rodent, cow, pig, sheep, horse, goat, dog or cat origin, and any other immune cell may be used. In some embodiments, the immune cell is human.

In some embodiments, the mammalian cell is an immune cell. As used herein, “immune cell” refers to a cell that plays a role in the immune response. Immune cells are of hematopoietic origin, and include T cells and natural killer (NK) cells. In some embodiments, the immune cell is a T cell. In some embodiments, the immune cell is a NK cell.

In some embodiments, the immune cell is obtained from an individual having or diagnosed as having cancer, a plasma cell disorder, or autoimmune disease. T cells can be obtained from a subject using standard techniques known in the field. For example, T cells can be isolated from peripheral blood taken from a donor or patient. T cells can be isolated from a mammal. Preferably, T cells are isolated from a human. In some embodiments, the immune cell is collected from a subject using leukapheresis.

In some embodiments, the immune cell is activated. In some embodiments, the immune cell is an activated T cell comprising a gRNA vector and a CAR vector. Methods of activating T cells are well known in the art. In some embodiments, activating n T cell comprises contacting the T cell with an anti-CD3 antibody. In some embodiments, activating n T cell comprises contacting the T cell with an anti-CD28 antibody. In some embodiments, activating a T cell comprises contacting the T cell with interleukin-2 (IL-2). In some embodiments, activating a T cell comprises contacting the T cell with Phytohemagglutinin (PHA). In some embodiments, activating a T cell comprises contacting the T cell with an anti-CD28 antibody and an anti-CD3 antibody. In some embodiments, activating a T cell comprises contacting the T cell with an anti-CD3 antibody and interleukin-2 (IL2). In some embodiments, activating a T cell comprises contacting the T cell with an anti-CD3 antibody, interleukin-2 (IL2) and Phytohemagglutinin (PHA). In some embodiments, any one of an anti-CD3 antibody, an anti-CD28 antibody, interleukin-2 (IL-2) and/or Phytohemagglutinin (PHA) are soluble when activating a T cell. In some embodiments, activating a T cell comprises contacting the T cell with an anti-CD3 antibody, interleukin-2 (IL2) and Phytohemagglutinin (PHA). In some embodiments, any one of an anti-CD3 antibody, an anti-CD28 antibody, interleukin-2 (IL-2) and/or Phytohemagglutinin (PHA) are fixed to a solid medium (e.g., a cell plate) when activating a T cell.

In some embodiments, the immune cell (e.g., T cell) does not comprise a DNA sequence encoding a CRISPR protein (e.g., Cas9).

In some embodiments, a plurality of immune cells (e.g., T cell or NK cells) are transfected with (1) a CAR vector and (2) a gRNA library as described herein to produce a mutant CAR-T cell library. Methods of immune cell transfection are well known in the art as described in Fus-Kujawa et al., “Frontiers in Bioengineering and Biotechnology (2021): 634, which is incorporated by reference in its entirety.

One advantage of using separate vectors to encode the CAR and the gRNA polynucleotides is the ability to control the multiplicity of infection (MOI) of the CAR and gRNA separately. Multiplicity of infection may refer to how many of a given vector enter into a cell. For example, and MOI of 1 indicates that the average number of a given vector per T cell in the mutant CAR-T cell library would be 1. In some embodiments, an MOI may be a target MOI based on the amount of vector transfected. In some embodiments, CAR-T cell efficacy may be dependent on the amount of CAR expressed in the cell. CAR expression may be controlled by increasing the number of CAR vector transfected per cell (e.g., increasing the MOI). However, in some embodiments, the skilled person may want to increase the MOI of the CAR vector but not the MOI of the gRNA vector because when more than one gRNA vector is transfected per cell then confounding results can be returned from having multiple different mutation made in the same cell. Therefore, in some embodiments, the MOI of the gRNA vector is less than 1 and the MOI of the CAR vector is greater than 1. In some embodiments, the gRNA library is transfected into the plurality of T cells at a multiplicity of infection (MOI) between 0.1-1, 0.2-1, 0.3-1, 0.4-1, 0.5-1, 0.6-1, 0.7-1, 0.8-1, 0.3-1, 0.4-0.9, 0.5-0.8, or 0.6-0.07. In some embodiments, the MOI of the gRNA vector is less than 1. In some embodiments, the MOI of the CAR vector is at least 1 (e.g., at least 2, at least 3, at least 4, at least 5, at least 10, at least 15 or at least 20). In some embodiments, the MOI of the CAR vector is between 1-3, 1-5, 1-10, 1-20, 5-10, 5-15, 5-20, or 10-20.

A “mutant CAR-T cell library” may refer to a two or more CAR-T cells, wherein at least two of the two or more CAR-T cells comprises a CAR (e.g., a CAR vector) and a gRNA vector, wherein the at least two of the two or more CAR-T cells comprises gRNA vectors encoding different guide RNAs. In some embodiments, at least 2 (e.g., at least 3, at least 4, at least 5, at least 6, at least 7, at least 8, at least 9, at least 10, at least 15, at least 20, at least 25, at least 50, at least 100, or at least 135) CAR-T cells of the library each comprise a different gRNA polynucleotide comprising a homology region that is complementary to a different gene sequence of any one of SEQ ID NOs: 1-135 or a variant thereof. In some embodiments, the CAR-T cell library comprises a gRNA vector described herein. In some embodiments, a CAR-T cell of the mutant CAR-T cell library comprises any one of SEQ ID NOs: 236-1315. In some embodiments, the mutant CAR-T cell library comprises any one of the compositions comprising gRNA polynucleotides described herein. In some embodiments, the mutant CAR-T cell library comprises at least 2 (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 50, at least 75, at least 100, at least 250, at least 500, at least 750, or at least 1000) of the gRNA homology region sequences of any one of SEQ ID NOs: 236-1315. In some embodiments, the mutant CAR-T cell library comprises SEQ ID NOs: 236-1315.

### Methods

In some aspects, the disclosure provides a method of producing a mutant immune cell library. In some embodiments, the immune cell library comprises T cells (a mutant CAR-T cell library). In some embodiments, the immune cell library comprises NK cells (a mutant CAR-NK cell library). In some embodiments, cells of the library comprise a CAR or vector encoding a CAR (e.g., a mutant CAR-T cell library or mutant CAR-NK cell library).

### Producing Mutant CAR-T Cell Libraries

In some aspects, the disclosure provides a method of producing a mutant CAR-T cell library. In some embodiments, the method comprises: (a) obtaining CAR-T cells (e.g., CAR-T cells previously made), and (b) transfecting the T cells with the gRNA vector library (e.g., as described herein). In some embodiments, the obtained CAR-T cells have been previously activated. In some embodiments, the method comprises activating the CAR-T cells. In some embodiments, the CAR-T cells express a CRISPR protein. In some embodiments, the method further comprises introducing a CRISPR protein encoding mRNA or a CRISPR protein into the CAR-T cells (e.g., by electroporation).

In some embodiments, the method comprises: (a) activating T cells (e.g., as described above (e.g., using anti-CD3 and anti-CD28 antibodies)), (b) transfecting the T cells with the gRNA vector library (e.g., as described herein) (c) transfecting the T cells with the CAR vector (e.g., as described herein), and (d) introducing a CRISPR protein encoding mRNA or a CRISPR protein into the T cell (e.g., by electroporation).

In some embodiments, the CRISPR protein is any suitable CRISPR protein for mutating DNA in CAR-T cells. In some embodiments, the CRISPR protein is any CRISPR protein described herein. In some embodiments, introducing a CRISPR protein encoding mRNA or a CRISPR protein into the T cell is performed 1-10 days after transfection of the gRNA vector and/or the CAR vector. In some embodiments, introducing a CRISPR protein encoding mRNA or a CRISPR protein into the T cell is performed 3-7 days after transfection of the gRNA vector and/or the CAR vector. In some embodiments, introducing a CRISPR protein encoding mRNA or a CRISPR protein into the T cell is performed 1, 2, 3, 4, 5, 6, 7, 8, 9, 10, 11, 12, 13, 14, or 16 days after transfection of the gRNA vector and/or the CAR vector. In some embodiments, the CRISPR protein introduced is a CRISPR protein. In some embodiments, the CRISPR protein introduced is introduced as a ribonucleoprotein. In some embodiments, the CRISPR protein encoded on the gRNA vector or the CAR vector.

In some embodiments, the method further comprises collecting T cells from a subject, e.g., that may then be modified using, e.g., one or more (e.g., all) of steps (a)-(d) described above. As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. In some embodiments, the subject is a human. In some embodiments, the subject is laboratory model organisms (e.g., a mouse). The terms, “individual,” “patient,” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female. A subject can be one who has been previously diagnosed with or identified as suffering from or having a condition in need of treatment (e.g., a pancreatic cancer, a lung cancer, an ovarian cancer, endometrial cancer, biliary cancer, gastric cancer, or mesothelioma or another type of cancer expressing an antigen target by the antigen binding domain of the CAR) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors. A “subject in need” of treatment for a particular condition can be a subject having that condition, diagnosed as having that condition, or at risk of developing that condition.

In some embodiments, the subject has been diagnosed with cancer. “Cancer” as used herein can refer to a hyperproliferation of cells whose unique trait, loss of normal cellular control, results in unregulated growth, lack of differentiation, local tissue invasion, and metastasis. Exemplary cancers include, but are not limited to, glioblastoma, prostate cancer, glioma, leukemia, lymphoma, multiple myeloma, or a solid tumor, e.g., lung cancer and pancreatic cancer. Nonlimiting examples of leukemia include acute myeloid leukemia (AML), chronic myeloid leukemia (CML), acute lymphocytic leukemia (ALL), and chronic lymphocytic leukemia (CLL). In some embodiments, the cancer is ALL or CLL. Non-limiting examples of lymphoma include diffuse large B-cell lymphoma (DLBCL), follicular lymphoma, small lymphocytic lymphoma (SLL), mantle cell lymphoma (MCL), marginal zone lymphomas, Burkitt's lymphoma, hairy cell leukemia (HCL), and T cell lymphoma (e.g., peripheral T cell lymphoma (PTCL), including cutaneous T cell lymphoma (CTCL) and anaplastic large cell lymphoma (ALCL)). In some embodiments, the cancer is DLBCL or follicular lymphoma. Non-limiting examples of solid tumors include adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, and Wilms tumor. In some embodiments, the cancer expresses mesothelin. In some embodiments, the cancer in uterine cancer. Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

In some embodiments, the T cells are collected using any suitable method. In some embodiments, the T cells are collected from the subject. In some embodiments, the T cell are collected from a healthy subject. In some embodiments, the T cells are collected by in vitro production (e.g., from induced pluripotent stem cells or embryonic stem cells). In some embodiments, the T cell are collected from a subject using Leukapheresis.

In some embodiments, the method further comprises purifying T cells that comprise a CAR vector and comprise a gRNA vector. In some embodiments, the methods of purifying may be performed based on the selectable molecule (e.g., reporters) as described above. For example, the CAR vector may express a selectable marker (e.g., CD34t) and the gRNA vector may express a selected marker (e.g., knockout of CD3). In some embodiments, purification can be performed using anti-body based magnetic selection. In some embodiments, purification can be performed using flow cytometry. In some embodiments, purification can be performed using CliniMACS In some embodiments, CAR-T cells with CRISPR activity are purified based on knockout of a gene for negative selection (e.g., CD3). In some embodiments, CAR-T cells expressing a CAR are selected for based on positive selection (e.g., expression CD34). In some embodiments, the success of transduction may be determined using flow cytometry based on the number of CD34+CD-LNGF+ cells.

In some embodiments, the gRNA vector library is transfected into the CAR-T cells with a MOI as described herein. In some embodiments, the CAR vector is transfected with an MOI as described herein.

In some embodiments, the present application discloses a mutant CAR-T cell library produced using any of the methods described herein.

### Identifying CRISPR Effects on CAR-T Cell Function.

In some embodiments, disclosure provides a method for identifying mutated (e.g., by CRISPR) CAR-T cells that have increased efficacy.

In some aspects the present application discloses a method of identifying gRNA polynucleotides associated with CAR-T cell efficacy in vivo comprising: (a) administering to a subject a mutant CAR-T cell library (e.g., a mutant CAR-T cell library disclosed herein or produced by a method described herein), (b) collecting one or more samples comprising a plurality of mutant CAR-T cells from the subject (e.g., via leukapheresis), (c) sequencing the gRNA polynucleotides from the mutant CAR-T cells collected in (b) (e.g., using any reasonable method, for example Illumina sequencing), and (d) evaluating the change in relative abundance of each gRNA polynucleotide based on the sequencing in (c). For example, a gRNA associated with CAR-T cell efficacy is a gRNA that induces a mutation which increases CAR-T cell proliferation and/or tumor toxicity. The skilled person will understand that an increase in relative abundance of a given gRNA after administration of a mutant CAR-T cell library to a subject indicates that the given gRNA causes a mutation that increases CAR-T cell efficacy (e.g., increase persistence).

The skilled person will understand that mutant CAR-T cells (e.g., of the mutant CAR-T cell library) may replicate in the subject. Thus, the plurality of mutant CAR-T cells collected in step b above may comprise mutant CAR-T cells that were administered to the subject and/or descendants of mutant CAR-T cells that were administered to the subject.

In some embodiments, the methods described herein relate to treating a subject having or diagnosed as having cancer, a plasma cell disease or disorder, or an autoimmune disease or disorder with a immune cell including any of the CAR polypeptides (and optional antibody reagents or cytokines) described herein, or a nucleic acid encoding any of the CAR polypeptides (and optional antibody reagents or cytokines) described herein. The CAR-T cells described herein include immune cells including any of the CAR polypeptides (and optional antibody reagents or cytokines) described herein, or a nucleic acid encoding any of the CAR polypeptides (and optional antibody reagents or cytokines) described herein.

The compositions described herein can be administered to a subject having or diagnosed as having a condition (e.g., cancer). As used herein, a “condition” refers to a cancer, a plasma cell disease or disorder, or an autoimmune disease or disorder. Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g., the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

In some embodiments, the methods described herein include administering an effective amount of activated CAR-T cells (e.g., a mutant CAR-T cell library) described herein to a subject to identify mutant CAR-T cells that have increased efficacy. In some embodiments, the methods described herein include administering an effective amount of activated CAR-T cells (e.g., a mutant CAR-T cell library) described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In some embodiments, the compositions described herein are administered systemically or locally. In a preferred embodiment, the compositions described herein are administered intravenously. In another embodiment, the compositions described herein are administered at the site of a tumor.

The term “effective amount” as used herein may refers to the number of immune cells (e.g. activated CAR-T cells or CAR-NK cells) needed to alleviate at least one or more symptom of the disease or disorder, and relates to a sufficient amount of the cell preparation or composition to provide the desired effect. The term “therapeutically effective amount” therefore may refer to an amount of activated immune cells (e.g., CAR-T cells or NK-cells) that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of activated CAR-T cells, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

Modes of administration can include, for example intravenous (iv) injection or infusion. The compositions described herein can be administered to a patient transarterially, intratumorally, intranodally, intraperitoneally or intramedullary. the In some embodiments, the compositions of T cells may be injected directly into a tumor, lymph node, or site of infection. In some embodiments, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the cells of interest, e.g., T cells. These T cell isolates can be expanded by contact with an artificial APC, e.g., an aAPC expressing anti-CD28 and anti-CD3 CDRs, and treated such that one or more CAR constructs of the technology may be introduced, thereby creating a CAR-T cell. These methods may further comprise transfecting the CAR-T cells with a gRNA library as described herein.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and

adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

In some embodiments, the mutant CAR-T cell library administered to the subject comprises a genome-wide CAR-T cell mutant library. A genome-wide CAR-T cell mutant library may refer to a plurality of CAR-T cells that have been transfected with a gRNA library that comprises at least one guide RNA targeting each gene in the genome of the CAR-T cells. In some embodiments, a genome-wide CAR-T cell mutant library comprises a plurality of CAR-T cells that have been transfected with a gRNA library that comprises at least one guide RNA targeting each protein encoding gene in the genome of the CAR-T cells. Genome-wide T cell CRISPR libraries and methods of use in vitro cells systems are described in Shifrut et al., Cell 175.7 (2018): 1958-1971.

In some embodiments, the mutant CAR-T cell library comprises negative control CAR-T cells. In some embodiments, the negative control CAR-T cells comprises a CAR vector as described herein and a negative control gRNA as described herein. In some embodiments, the negative control CAR-T cells comprise an empty CAR vector that does not encode a CAR. In some embodiments, the negative control CAR-T cells comprise a CAR that does not comprises an antigen binding domain. In some embodiments, the negative control CAR-T cells comprise a CAR that comprises an antigen binding domain that does not bind to a protein expressed by the subject.

In some embodiments, the subject is any suitable subject including a subject described herein. In some embodiments, the subject is human (e.g., a human subject as described herein). In some embodiments, the subject has cancer (e.g., a cancer described herein). In some embodiments, the cancer is selected from the group consisting of ovarian cancer, pancreatic cancer, lung cancer, prostate cancer, breast cancer, AML, multiple myeloma, or B cell lymphomas)

In some embodiments, the CAR comprises an antigen binding domain that binds to an antigen expressed by the cancer (e.g., an antigen binding domain as described herein). In some embodiments, the CAR comprises an antigen binding domain that binds to any one of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain. In some embodiments, the antigen binding domain binds to mesothelin.

In some embodiments, the one or more samples collected from the subject are collected for any reasonable tissue or bodily fluid using any reasonable means. In some embodiments, the one or more samples are collected from a bodily fluid (e.g., cerebrospinal fluid, blood or a blood product). In some embodiments, the blood product is serum or plasma. In some embodiments, the one or more samples are collected from bone marrow. In some embodiments, the one or more samples are collected from a tissue (e.g., heart, lung, liver, pancreas, brain, skin, intestine, kidney, spleen, lymph node, thyroid, muscle, fat, gull bladder, or tonsils).

In some embodiments, sample collection is performed using any reasonable means, including, but not limited to venipuncture, apheresis, tissue biopsy or resection of tumor.

In some embodiments, one sample is collected from the subject. In some embodiments, 1, 2, 3, 4, 5, 6, 7, 8, 9, 10, 11, 12, 13, 14, 15, or more samples (e.g., blood samples) are collected from the subject. In some embodiments, at least 2 (e.g., at least 3, at least 4, at least 5, at least 6, at least 7, at least 8, at least 9, at least 10, at least 11, at least 12, at least 13, at least 14, or at least 15) samples (e.g., blood samples) are collected from the subject. In some embodiments, 1-2, 1-2, 1-3, 1-4, 1-5, 1-6, 1-7, 1-8, 1-9, 1-10, 1-15, 2-3, 2-5, 2-7, 2-10, 2-12, 2-15, 3-5, 3-7, 3-10, 3-12, or 3-15 samples are collected from a subject. In some embodiments, at least 5 samples are collected via venipuncture, apheresis, or resection of tumor.

In some embodiments, samples are collected from the subject at one or more of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or day 31 after step (a). In some embodiments, samples are collected from the subject at one or more of week 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 after step (a). In some embodiments, samples are collected from the subject at one or more of month 1, 2, 3, 4, 5, 6, 7, 8, 9, 10, 11, 12 after step (a). In some embodiments, samples are collected from the subject at day 3, day 7, day 10, day 14, day 21, day 28, month 2, month 3, and month 6 after step (a).

In some embodiments, the method further comprises extracting mutant CAR-T cells from the sample(s) prior to sequencing. Methods for isolating CAR-T cells from samples (e.g., bodily fluids) are well known in the art. For example, leukapheresis is a known method for extracting CAR-T cell from a subject. Other methods may include immune precipitation, immunomagnetic selection, microfluidics, and flow cytometry sorting. These methods are described in Chiu et al., Scientific Reports 9.1 (2019): 1-10, which is incorporated by reference in its entirety. In some embodiments, extraction of mutant CAR-T cell from a sample is performed based on cell surface markers expressed by the mutant CAR-T cell. In some embodiments, extraction is performed by isolating T cells that are CD3 negative, CD34 positive, and optionally LNGFR positive.

In some embodiments, the method further comprises extracting genomic DNA from the mutant CAR-T cells. In some embodiments, genomic DNA is extracted from a single mutant CAR-T cell or a clonal population of a mutant CAR-T cell. In some embodiments, the method comprises sequences the extracted genomic DNA to determine the mutation caused by the CRISPR guide RNA in a given CAR-T cell. Alternatively, in some embodiments, the method comprises using primers to amplify the region of the genome expected to comprise a CRISPR induced mutation (e.g., based on the sequence of the gRNA homology region), and sequencing that region.

In some embodiments, the method further comprising isolating the RNA from the mutant CAR-T cells. In some embodiments, the RNA is isolated from single mutant CAR-T cells (e.g., for single cell RNA sequencing). In some embodiments, the isolated RNA from the mutant CAR-T cell is sequenced (e.g., using illumine sequencing).

In some embodiments, the method comprises sequencing the gRNA polynucleotides in the mutant CAR-T cells extracted from the subject. In some embodiments, the method involves extracting DNA from the mutant CAR-T cells. The skilled person will understand that DNA can be extracted from a pool of mutant CAR-T cells collected from the subject. In other words, in some embodiments, DNA from each CAR-T cell does not need to be separately isolated from each mutant CAR-T cell collected from the subject to determine the effects of a CRISPR mutation on CAR-T cell efficacy. Rather, the guide RNA homology region sequences may be used to identify which gene is expected to be mutated by CRISPR and the expected location of the mutation in the gene because the guide RNA homology regions may have a sequence that is complementary to a specific region of a specific gene.

Thus, in some embodiments, the method further comprises using primers to amplify the gRNA polynucleotides of the mutant CAR-T cells to produce amplicons of any thereof, and these amplicons are used for sequencing. In some embodiments, the gRNA polynucleotides amplified are genomic DNA, vector DNA, or RNA. In some embodiments, universal primers may be used to amplify all of the guide RNA polynucleotides in the mutant CAR-T cell library. In some embodiments, the universal primers comprising a universal forward primer and a universal reverse primer. In some embodiments, the forward and reverse primer are designed to bind to regions of the gRNA vector and/or the gRNA polynucleotide that are the same. For example, in some embodiments, the universal forward primer binds to the gRNA polynucleotide upstream of the homology region. In some embodiments, the universal reverse primer binds to the gRNA polynucleotide downstream of the homology region. In some embodiments, the universal forward primer comprises the sequence of SEQ ID NO: 1324. In some embodiments, the forward primer and reverse primer comprise phosphorothioate modifications. In some embodiments, the universal forward primer comprises a stagger sequence (e.g., a sequence of variable length in the primer to improve resolution of next-generation sequencing). Stagger sequences are also known in the art, e.g., as described in Wohlhieter et al., STAR Protocols 2.2 (2021): 100390. In some embodiments, the stagger sequence comprises any one of C, GC, AGC, CAAC, TGCACC, ACGCAAC, and TGAAGACCC. In some embodiments, the universal reverse primer comprises a stagger sequence.

In some embodiments, the universal reverse primer comprises the sequence of SEQ ID NO: 1325.

In some embodiments, the gRNA polynucleotides or amplicons thereof are sequenced. Sequencing of the gRNA polynucleotides, amplicons thereof, or any other sequencing described herein may be performed using any suitable method or apparatus, including, but not limited to Next-generation sequencing, Illumina™ sequencing, Ion Torrent™ sequencing, PacBio™ sequencing, nanopore sequencing, 454 sequencing, sanger sequencing, and SoLid™ sequencing.

In some embodiments, the method comprises evaluating the change in relative abundance of each gRNA polynucleotide based on the sequencing. In some embodiments, relative abundance of each gRNA polynucleotide is based on the number of time a given gRNA polynucleotide is observed is the sequencing data before the mutant CAR-T cell library is administered to the patient and after the mutant CAR-T cell library is extracted from the patient. In some embodiments, in some embodiments, the relative abundance of a given guide RNA in the sequencing data polynucleotide is determined over time (e.g., in each sample collected). The relative abundance of a given guide RNA may be indicative of the efficacy of the CAR-T cell.

In some embodiments, the method comprising determining a fold change value by comparing the average gRNA polynucleotide abundance for any given gene in the post-infusion sample (extracted from a subject) to its abundance in the pre-infusion sample (prior to administration to the subject). In some embodiments, the threshold for identifying significant fold changes in gRNA polynucleotide abundance is based on z-score normalization the distribution of fold change values. In some embodiments, changes of greater than 2 standard deviations from the mean of the z-score distribution are considered significant.

In some embodiments, the efficacy of each mutant CAR-T cell is evaluated based on persistence in vivo and tumor cytotoxicity. The terms “persistence” or “CAR-T cell persistence” as used herein refer to the ability of a CAR-T cell to stay activated, continue to proliferate, and/or not die. Persistence may be measured based on the relative abundance of a mutant CAR-T cell overtime because proliferation and death of a mutant CAR-T cell would be expected to alter the relative abundance of the mutant CAR-T cell. Thus, a mutant CAR-T cell with increased relative abundance may also have increased persistence.

In some embodiments, persistence in vivo and tumor cytotoxicity are measured using flow cytometry and PCR. In some embodiments, quantifying persistence is performed by calculating the relative change in each gRNA over time. In some embodiments, quantifying persistence is performed by calculating the relative change in each gRNA over time compared to a gRNA polynucleotide from a negative control CAR-T cell. In some embodiments, quantifying persistence is performed by calculating the relative change in each gRNA over time across at least 3 timepoints and calculating a slope of relative change. In some embodiments, tumor cytotoxicity is quantified using clinical imaging and/or bone marrow evaluation.

In some embodiments, mutant CAR-T cell persistence is determined based on relative abundance then mutant CAR-T cells with high relative abundance are tested for cancer killing efficacy (e.g., in vitro or in vivo).

In some embodiments, efficacy is measured for the entire mutant CAR-T Cell library that is administered to the patient. For example, if the patient show sign of improvement (e.g., decreased tumor or cancer burden, increased lifespan, etc.) after administration of a mutant CAR-T cell library then the library as a whole may be efficacious.

### Producing Mutant NK Cell Libraries

In some embodiments, the methods described herein to produce mutant CAR-NK cell libraries can be adapted to produce mutant natural killer cell libraries. In some embodiments, the method comprising transfecting a gRNA vector library into a plurality of NK cells (e.g., CAR-NK cells) extracted from a subject (e.g., using a method described herein). In some embodiments, the NK cells are also transfected with a CAR vector as described herein.

In some aspects, the disclosure provides a method of producing a mutant CAR-NK cell library. In some embodiments, the method comprises: (a) obtaining CAR-NK cells (e.g., CAR-NK cells previously made), and (b) transfecting the CAR-NK cells with the gRNA vector library (e.g., as described herein). In some embodiments, the obtained CAR-NK cells have been previously activated. In some embodiments, the method comprises activating the CAR-NK cells. In some embodiments, the CAR-NK cells express a CRISPR protein. In some embodiments, the method further comprises introducing a CRISPR protein encoding mRNA or a CRISPR protein into the CAR-NK cells (e.g., by electroporation).

In some aspects, the present application discloses a method of producing a mutant CAR-NK cell library comprising: (a) activating NK cells (e.g., as described above (e.g., using anti-CD3 and anti-CD28 antibodies)), (b) transfecting the NK cells with the gRNA vector library as described above (c) transfecting the NK cells with the CAR vector of any one of claims as described above, and (d) introducing a CRISPR protein encoding mRNA or a CRISPR protein into the NK cell (e.g., by electroporation).

In some embodiments, the CRISPR protein is any suitable CRISPR CAR for mutating DNA in CAR-NK cells. In some embodiments, the CRISPR protein is any CRISPR protein described herein. In some embodiments, introducing a CRISPR protein encoding mRNA or a CRISPR protein into the NK cell is performed 1-10 days after transfection of the gRNA vector and/or the CAR vector. In some embodiments, introducing a CRISPR protein encoding mRNA or a CRISPR protein into the NK cell is performed 3-7 days after transfection of the gRNA vector and/or the CAR vector. In some embodiments, the CRISPR protein introduced is a CRISPR Cas9 protein. In some embodiments, the CRISPR protein introduced is introduced as a ribonucleoprotein. In some embodiments, the CRISPR protein mRNA is expressed from the gRNA vector or the CAR vector.

In some embodiments, the method further comprises collecting NK cells from a subject e.g., that may then be modified using, e.g., one or more (e.g., all) of steps (a)-(d) described above (e.g., a subject as described above).

In some embodiments, the NK cells are collected using any suitable method (e.g., including any suitable method described above).

In some embodiments, the method further comprises purifying NK cells that comprise a CAR vector and comprise a CRISPR induced gene mutation (e.g., including any suitable purifying method described above).

In some embodiments, the gRNA vector library is transduced into the CAR-NK cells with a MOI as described above. In some embodiments, the CAR vector is transduced with an MOI as described above.

In some embodiments, the present application discloses a mutant CAR-NK cell library produced using any of the methods described herein.

### Identifying CRISPR Effects on CAR-NK Cell Function.

In some embodiments, the disclosure provides a methods for identifying mutated (e.g., by CRISPR) CAR-NK cells that have increased efficacy.

In some aspects the present application discloses a method of identifying gRNA polynucleotides associated with CAR-NK cell efficacy in vivo comprising: (a) administering to a subject a mutant CAR-NK cell library (e.g., a mutant CAR-NK cell library disclosed herein or produced by a method described herein), (b) collecting one or more samples comprising a plurality of mutant CAR-NK cells from the subject (e.g., via leukapheresis), (c) sequencing the gRNA polynucleotides from the mutant CAR-NK cells collected in (b) (e.g., using any reasonable method, for example Illumina sequencing), and (d) evaluating the change in relative abundance of each gRNA polynucleotide based on the sequencing in (c). For example, a gRNA associated with CAR-NK cell efficacy is a gRNA that induces a mutation which increases CAR-NK cell proliferation and/or tumor toxicity. The skilled person will understand that an increase in relative abundance of a given gRNA after administration of a mutant CAR-NK cell library to a subject indicates that the given gRNA causes a mutation that increases CAR-NK cell efficacy (e.g., increase persistence).

The skilled person will understand that mutant CAR-NK cells (e.g., of the mutant CAR-NK cell library) may replicate in the subject. Thus, the plurality of mutant CAR-NK cells collected in step b above may comprise mutant CAR-NK cells that were administered to the subject and/or descendants of mutant CAR-NK cells that were administered to the subject.

The CAR-NK cells described herein include immune cells including any of the CAR polypeptides (and optional antibody reagents or cytokines) described herein, or a nucleic acid encoding any of the CAR polypeptides (and optional antibody reagents or cytokines) described herein.

In some embodiments, the methods described herein include administering an effective amount of activated CAR-NK cells (e.g., a mutant CAR-NK cell library) described herein to a subject to identify mutant CAR-NK cells that have increased efficacy. In some embodiments, the methods described herein include administering an effective amount of activated CAR-NK cells (e.g., a mutant CAR-NK cell library) described herein to a subject in order to alleviate a symptom of the condition.

In some embodiments, the compositions of NK cells may be administered to subject as described above (e.g., in the same or similar manner as performed using mutant CAR-T cells).

In some embodiments, the mutant CAR-NK cell library administered to the subject comprises a genome-wide CAR-NK cell mutant library (e.g., a genome-wide generated using a genome wide gRNA vector library as described above for mutant CAR-T cells).

In some embodiments, the CAR of the CAR-NK cell comprises an antigen binding domain that binds to an antigen expressed by the cancer (e.g., an antigen binding domain as described herein). In some embodiments, the CAR comprises an antigen binding domain that binds to any one of CD19, CD79b, TACI, BCMA, MUC1, MUC16, B7H3, mesothelin, CD70, PSMA, PSCA, EGFRVIII, claudin6, binds to any pair of CD19/CD79b, BCMA/TACI, or is a TriPRIL antigen binding domain. In some embodiments, the antigen binding domain binds to Mesothelin.

In some embodiments, the one or more samples collected from the subject are collected for any reasonable tissue or bodily fluid using any reasonable means as described above.

In some embodiments, method of collecting mutant CAR-NK cells from a subject, extracting genomic DNA and RNA from CAR-NK-cells, amplifying and/or sequencing gRNA polynucleotides, determining relative abundance, and determining CAR-NK-cell efficacy are described above in relation to CAR-T cells and may be similarly used with CAR-NK-cells.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### EXAMPLES

### Example 1: Vector Design for gRNA Vector and CAR Vector

Two different vectors were designed that together would provide CAR expression and positive selection using a clinically-feasible magnetic bead system, and expression of two guides, a suicide gene, and a selectable marker that would enable specific CRISPR/Cas9 knockout of two genes, one of which is to be used for negative selection with said clinically-feasible magnetic bead system.

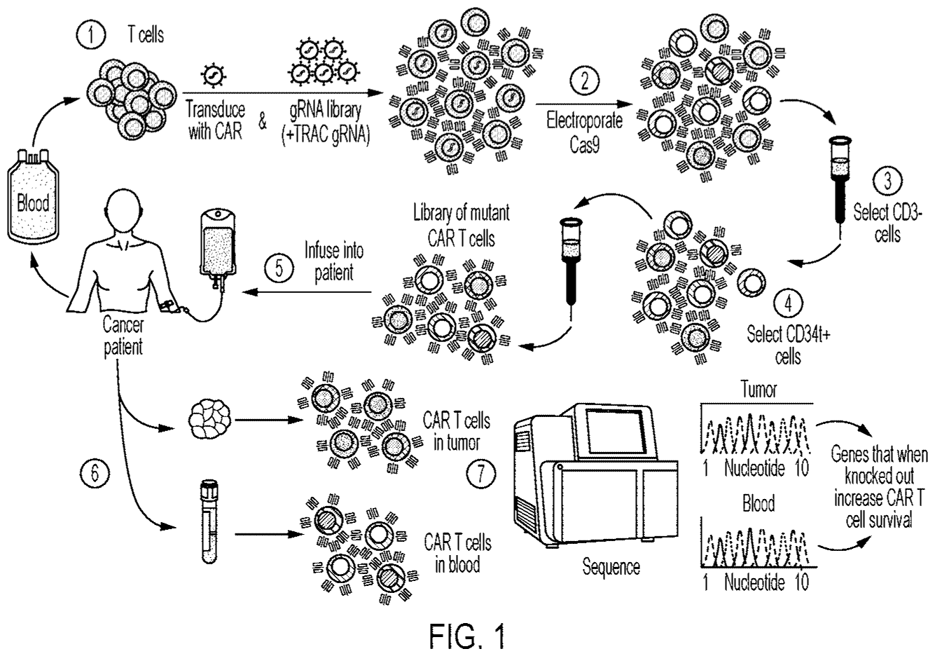

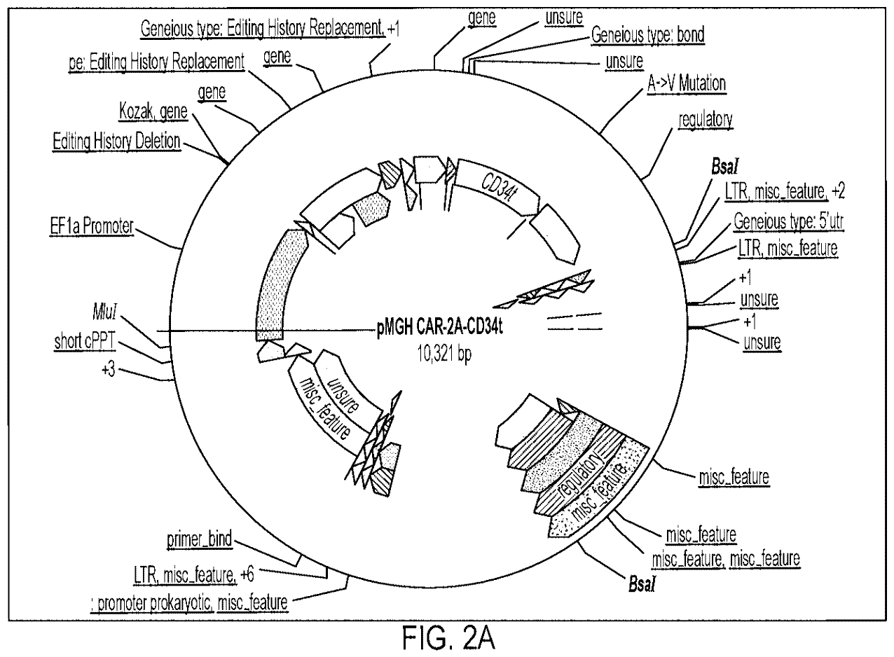

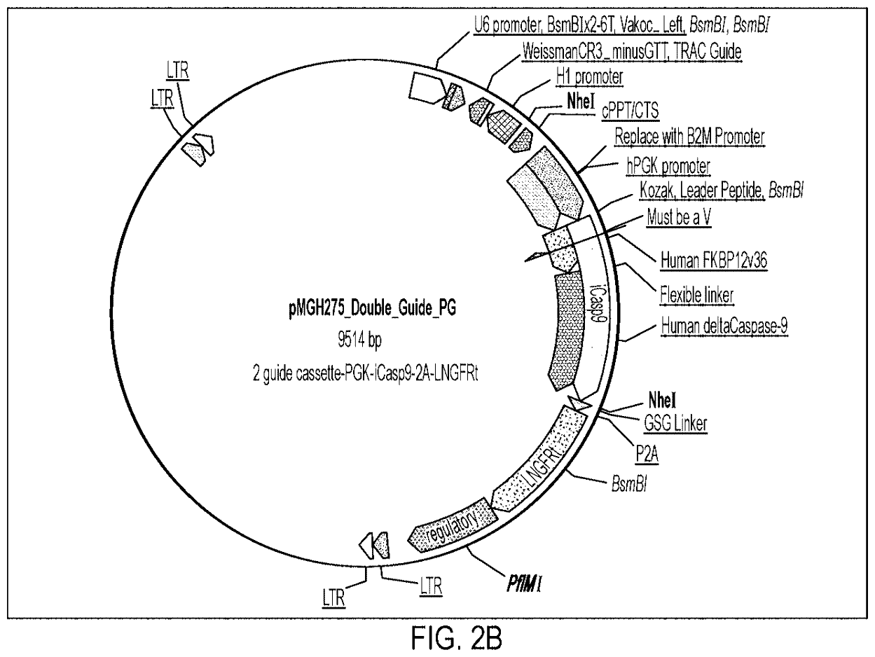

The CAR vector was composed of the backbone of a third-generation self-inactivating (SIN) lentiviral vector (FIG. 1A). A promoter (for example, EF1a) was used to drive expression of the CAR. A 2A ribosomal skip element follows the CAR sequence, and was followed by a reporter gene detectable and selectable by staining for surface expression (truncated CD34, which lacks an intracellular domain).

The double-guide vector backbone (e.g., gRNA vector) enabled the use of a library of guides. The vector backbone encoded 2 guides; one was static (TRAC) and enabled knockout of the TCR/CD3 complex on gene-edited T cells; the absence of CD3 enabled clinically-feasible negative selection, selecting the effector population in which successful transduction and gene editing with CRISPR/Cas9 occurred. In this exemplary embodiment, the guide RNAs on the vector were inverted; in another embodiment, the two guides are configured in a tandem manner (FIG. 1B).

EF1a and EF1a-short (EFS) were used for CAR and reporter expression. Expression of the reporter gene LNGFR and suicide gene was driven by a weaker promoter (PGK) to avoid overexpression of the suicide gene which could be leaky and trigger apoptosis of the cell product.

The CAR vector and double-guide vector are co-transduced into the same population of T cells. The final ‘active’ investigational product of interest is CAR+CD34+LNGFR+CD3− T cells. For enhanced safety, a suicide gene (for example, icaspase9) may be included in the dual-guide vector.

### Example 2: GRNA Library Design and Production

### Selection of Genes

Using genomic, transcriptional, and functional data from human and murine T cells, an initial list of 135 genes amenable to CRISPR/Cas9 knockout and hypothesized to improve CAR-T cell functions were selected (Table 2). These genes included checkpoint proteins (e.g., PD-1, encoded by PDCD1), as well as genes observed to be mutated in subjects having lymphoproliferative diseases or subjects with unusual responses to CAR-T cells (e.g., TET2 and cbl-b).

Determining sgRNA Library Size

Library size was calculated based on feasibility of manufacturing and dose of cells to be administered (typically 100×106), and the number of cells that can reasonably be recovered from a patient post-infusion (typically 50×106-100×106). Targeting the entire genome would result in suboptimal depth and a low signal:noise ratio. On the other hand, targeting <10 genes would be less informative as to which candidate gene knockouts are optimal. Targeting 100-500 genes, with redundancy in guides to enhance signal:noise ratio, provides depth of coverage, decreases randomness due to number of T cells recovered post-infusion, and yet presents a competitive system within and among different patients to rigorously assess the effects of a variety of candidate genes. The library also includes controls that target genes which are expected to have any effect on T cell biology.

sgRNA Library Design

For the 135 genes selected, a library of 8 sgRNAs per gene was designed using the dominant transcript ID in human T cells for each gene (e.g. encoded by SEQ ID NOs: 1-135). These transcripts were used to design all possible guides using CRISPick. A total of 34,318 guides were designed. These guides were scored for on-target activity using Rule Set 3 (RS3) with sequence and target information and the Chen2013 tracr (PMID: 24360272). The guides were also scored for off-target activity using Tier-agnostic 1 mismatch aggregated CFD scores. 2009 guides were filtered out based on cloning incompatibility (e.g., BsmBI sites) and polyTs. Five guides had >10000 off-target sites with 1 mismatch. 277 guides were filtered out that had off-targets in 58 tumor suppressor genes as defined by Lenoir et al. Nature communications 12.1 (2021): 1-15. 118 guides that were flagged for targeting a region of the genome with high variation in the human population were also filtered out. 5561 guides that targeted outside 2-85% protein window were also filtered out. To avoid promiscuous guides, 4771 guides with aggregated Cutting Frequency Determination (CFD) scores ≤0.27 were filtered out.

After all the filtering was applied, the top 8 guides per gene were selected by RS3 scores. This library has a total of 1080 gene targeting guides (Table 4). 100 validated intergenic controls were also included, bringing the total number of guides to 1180.

sgRNA Library Production

To generate the sgRNA library, oligos for each sgRNA are synthesized on a chip and then amplified using PCR primers containing BsmBI-compatible cloning sequences. The amplified oligo pool is ligated into the sgRNA vector described above and the ligation product is transformed into electrocompetent E. coli. Transformed E. coli are then plated on solid agar at a density to achieve 1000-fold representation of each sgRNA in the library and grown overnight. Colonies are scraped from plates and pooled sgRNA plasmid is extracted. The plasmid library is sequenced to confirm sgRNA representation and then retransformed into electrocompetent E. coli and stored frozen in glycerol media. Aliquots of glycerol-preserved E. coli are then grown overnight in liquid culture and plasmid is extracted from expanded E. coli. Pooled sgRNA library lentivirus prepared using standard 3rd generation lentiviral vector production.

### Example 3: Producing and Administering a Mutant CAR-T Cell Library

T cells are collected from human subjects via standard leukapheresis procedures. Peripheral blood mononuclear cells are isolated and T cells are purified. T cells are then activated using anti-CD3/anti-CD28 antibodies (optionally antibody-coated beads), and then transduced with both vectors; the MOI of the double-guide vector is capped at 1, and optionally is or approaches 0.5. The MOI of the CAR vector is as high as 20.

The Cas9 is introduced into CAR-T cells either in the form of mRNA encoding Cas9 or in the form of ribo-nucleoprotein (RNP), where soluble Cas9 protein is complexed with a non-targeting guide RNA with no predicted target site in the genome. Whether by mRNA or RNP, the Cas9 is electroporated into the T cells 3-7 days after lentiviral transduction.

The T cells are further expanded and then selected through a series of magnetic bead processes. First, the product is negatively selected for CD3 expression by using a system such as CliniMACS that eliminates T cells. The CD3-negative fraction is then positively selected for CD34 expression using a system such as CliniMACS. The CD34+CD3− cells considered the investigational product for infusion.

Flow cytometry analysis is used to determine the number of CAR+ cells based on CD34+ cells, and the number of successfully transduced and gene edited cells based on CD3 negative and LNGFR+.

CAR T cells are administered based on dose of CAR+ cells, in the range of 10×10⁶ (a likely suboptimal dose level) to 100×10⁶ (a typical dose level) to 300×10⁶ (the typical maximum tolerated dose for CAR T cells). The library representation is measured as baseline to evaluate in-manufacturing skewing, but not be part of the dose selection per se.

### Example 4: Determining Mutant CAR-T Cell Efficacy

A sample of mutated T cells is collected from the subject via venipuncture, apheresis, or resection of tumor according to a schedule of events on a clinical protocol (typically, day 3, day 7, day 10, day 14, day 21, day 28, month 2, month 3, and month 6). T cells are isolated via standard procedures. CAR+ T cells are purified based on expression of CD34, and analyzed for expression of CD3 and other T cell markers such as CD4 and CD8. Genomic DNA is isolated from purified CAR+ T cells for downstream analysis. The sgRNA region from CAR+ T cells is amplified using PCR primers specific for sequences flanking the sgRNA cassette containing the library. PCR amplicons are then sequenced using Nextgen sequencing to quantify the abundance of each individual sgRNA in the library and the abundance for each guide in post-infusion CAR+ T cells is compared to the abundance values in the pre-infusion CAR+ T cells.

This method also allows for single-cell transcriptomic profiling of the CAR-T cells isolated from patients with capture of the sgRNA to allow for linkage of gene deletion with transcriptional phenotyping. Standard droplet-based single cell whole-transcriptome analysis platforms such as 10× Genomics is used, combined with direct capture of the sgRNA sequence from cellular RNA using a primer set specific for the sgRNA region.

### Example 5: Knockout of Multiple Genes in Primary T Cells Using Double Guide Cassette Vector

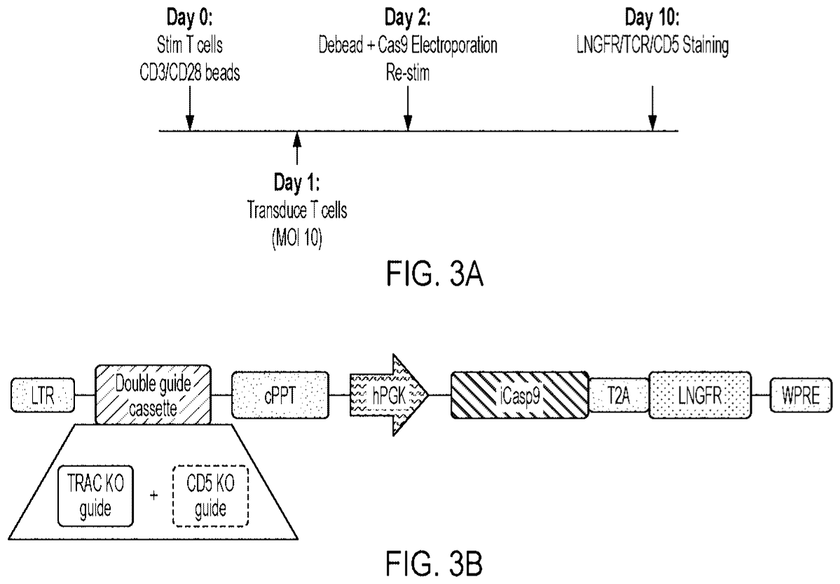

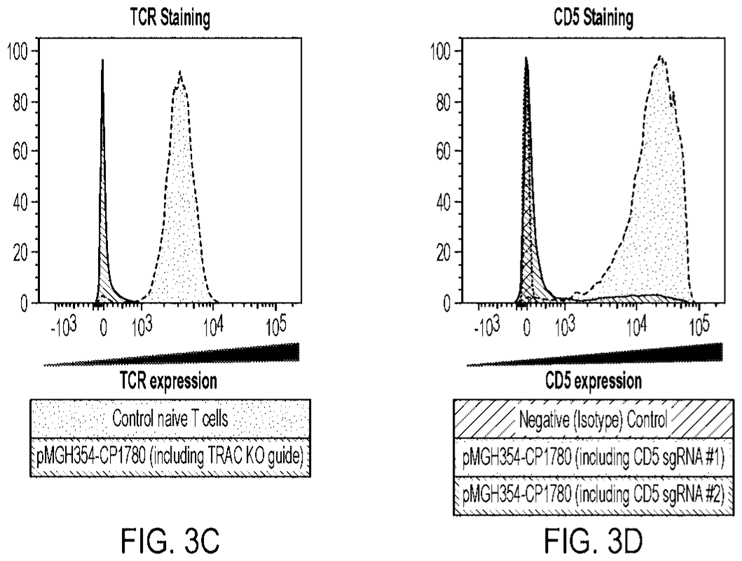

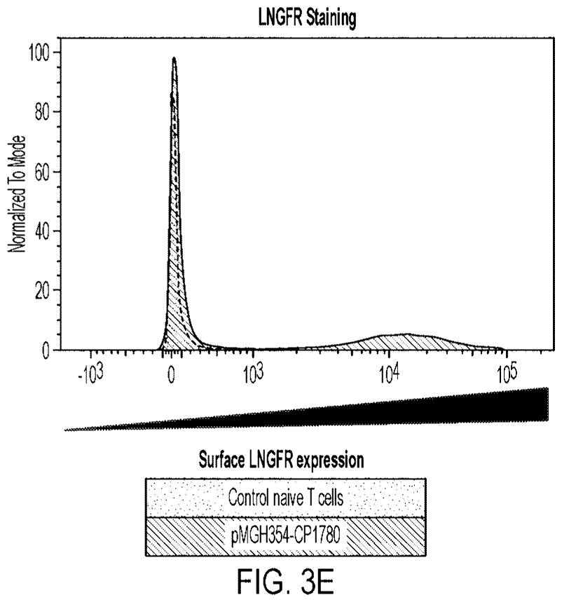

CD5 and TRAC genes were knocked in T cells out using a gRNA vector comprising two guide RNAs. Naïve T cells were activated using anti-CD3/CD28 beads (FIG. 3A). On day 1, activated T cells were transduced with the guide RNA vector which contained sgRNAs targeting CD5 and TRAC, an important component of the T cell receptor complex (TCR) (FIG. 3B). The next day, transduced cells were de-beaded (removing the anti-CD3/CD28 beads), electroporated with Cas9 and given fresh media containing IL-2 every two days. On day 10, transduced T cells were analyzed for LNGFR expression as well as TCR and CD5 knockout. (FIGS. 3C-3E). Naïve and transduced T cells were analyzed by flow cytometry after staining for cell surface expression of the TCR, CD5 and LNGFR. Results show untransduced naïve T cells maintain expression of the TCR (FIG. 1C). However, guide RNA vector transduced cells demonstrate knockout of the T cell receptor complex, resulting from expression of the TRAC targeting sgRNA and electroporation with Cas9. Two versions of the double guide cassette vector (pMGH354-CP1780) were tested (FIG. 3D). Each version contained a different sgRNA targeting CD5 (CD5 sgRNA #1 or CD5 sgRNA #2). Transduced T cells were stained for CD5 using fluorescent antibodies detecting CD5 and analyzed by flow cytometry. An isotype control, a similar fluorescent antibody which lacks specificity to CD5, was used as a negative control. Results demonstrate loss of CD5 surface expression in T cells transduced with CD5 sgRNA #2 but maintained expression of CD5 in T cells transduced with CD5 sgRNA #1. Overall, the strong CD5 knockout seen with CD5 sgRNA #2 shows appropriate function of the guide RNA vector. The maintained CD5 expression seen with CD5 sgRNA #1 suggests a poor functioning sgRNA and is not reflective of the guide RNA vector itself. Untransduced naïve T cells and gRNA vector transduced T cells were stained for expression of the LNGFR cell surface marker. Results show LNGFR expression in gRNA vector transduced T cells only (FIG. 3E).

### Example 6: Guide RNA Polynucleotide Abundance in Pooled Library Determined Via Sequencing

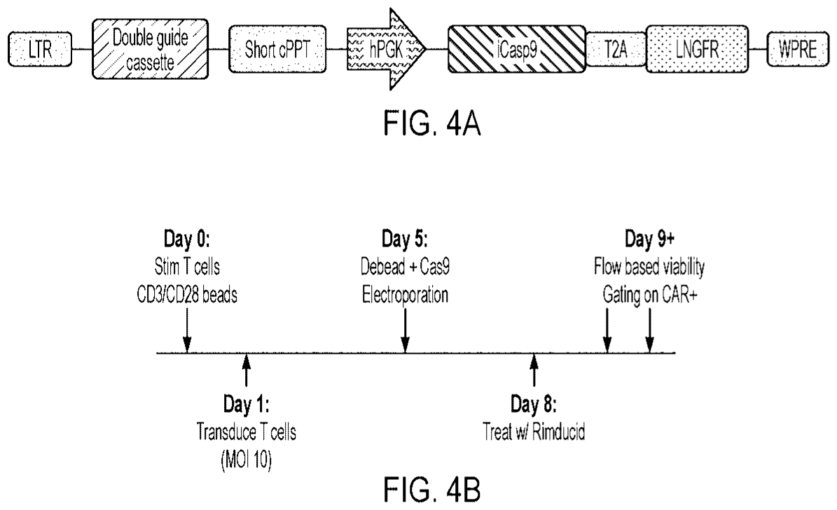

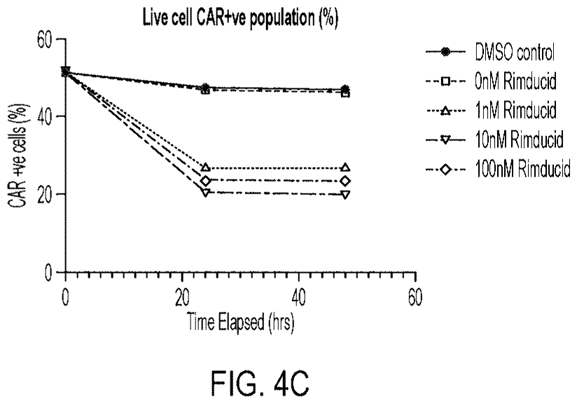

Naïve T cells were activated using anti-CD3/CD28 beads. On day 1, activated T cells were transduced with the double guide cassette vector (pMGH354-CP1780) which contained two sgRNAs, a drug-inducible Caspase-9 (iCasp9) safety switch and a truncated LNGFR selection marker (FIG. 4A-4B)). On day 5, transduced cells were de-beaded (removing the anti-CD3/CD28 beads), electroporated with Cas9 and replenished with fresh media containing IL-2. On day 8, pMGH354-CP1780 transduced T cells were left untreated (0 nM Rimducid), treated with vehicle only (DMSO control), or treated with varying concentrations of Rimducid which activates the iCasp9 safety switch. Each experimental condition was performed in triplicate.

Response to Rimducid was assessed by measuring a change in proportions of a guide RNA vector (CAR+ve) cells following treatment (FIG. 4C). CountBright™ absolute counting beads and a live/dead cell stain were mixed with the cell sample and assayed via flow cytometry at 24 and 48 hours after Rimducid treatment. By comparing the ratio of bead events to live cell events, absolute numbers and proportions of CAR+ve live cells in the sample was calculated. Results show maintained CAR+ve cell proportions in untreated and vehicle treated control groups. However, Rimducid treated groups demonstrate loss of CAR +ve cells over time. This shows appropriate expression and function of the iCasp9 safety switch in pMGH354-CP1780 transduced T cells.

### Example 7: CAR-T Cell CRISPR Library and Use in Humans Having Solid Tumors

Chimeric antigen receptor (CAR) T cells have transformed the treatment landscape for patients with hematologic malignancies, achieving durable complete remissions in ˜40% of patients with refractory lymphomas. However, some patients ultimately relapse, and CAR T cells have been relatively unsuccessful in patients with advanced solid tumors so far. While some of this acquired resistance is explained by the loss of the CAR target antigen, a major driver of treatment failure in both liquid and solid tumors is the inability of CAR T cells to persist and maintain sustained effector capacity in the face of persistent antigen expression, which is often referred to as “exhaustion.” This evolutionary program limits damage to the host during infection and autoimmune inflammation but creates a critical barrier to CAR T cell efficacy. In solid tumors, this program is exaggerated by the suppressive tumor microenvironment (1,2). Thus, it is critically important to define the genes and pathways that can enhance CAR T cell efficacy in both solid and liquid tumors in vivo, where the factors driving T cell dysfunction are at work.

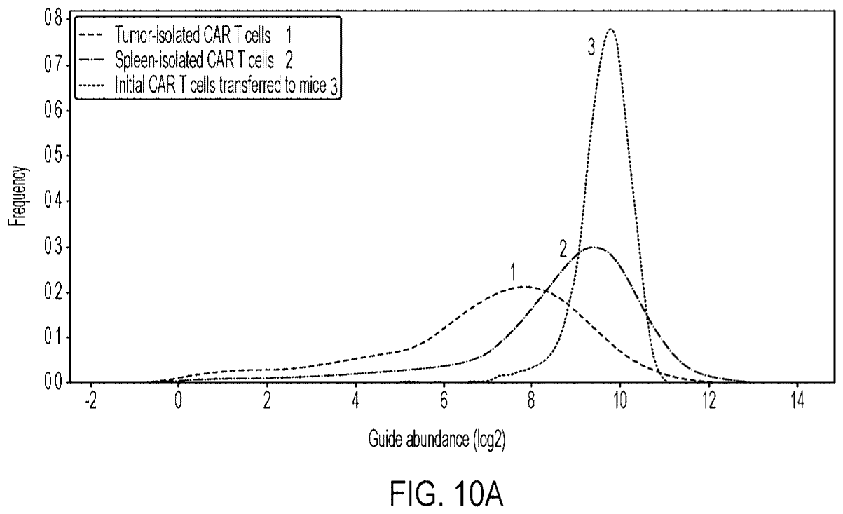

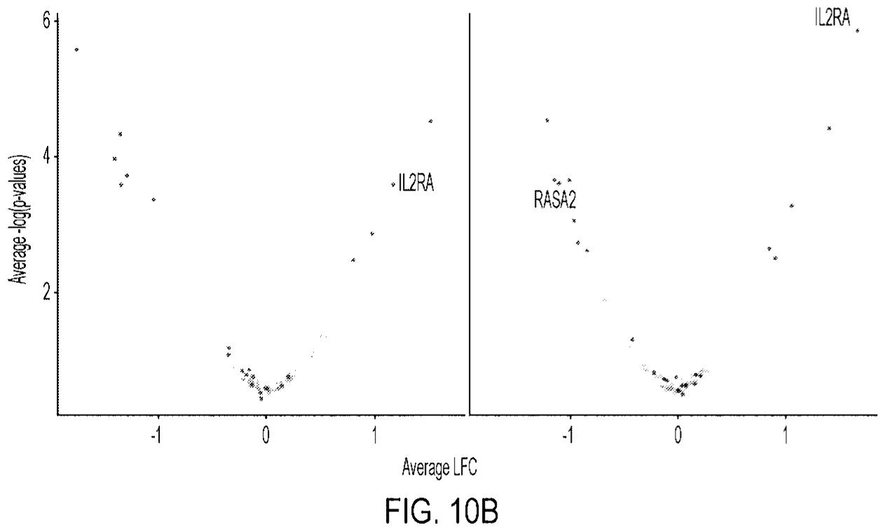

There are various genes and pathways that could theoretically be modulated to improve CAR-T cell responses. However, there are so many candidate genes that it will essentially be impossible for the field to validate each potential candidate individually in separate Investigational New Drugs (INDs) and Phase 1 clinical trials of CAR T cells. While data from in vitro and in vivo studies provide valuable insights about the factors that govern CAR T cell functionality, parallel in vitro and in vivo genetic screens in solid and liquid cancer models show that targets identified through in vitro screens do not always correlate with in vivo screens (3,4). Some of the target genes identified as regulators of T cell function in genetic screens in vitro, such as RASA2, have not been identified as top targets in in vivo screens disclosed herein (e.g., FIG. 10B) (5). Additionally, preclinical in vivo models of CAR T cell efficacy do not always predict performance in the clinic. Therefore, a genetic screen of genes regulating CAR T cell function in human patients may be the only way to identify genes that will improve clinical performance. To fill this need, this Example implements screens of selected target genes in CAR T cells given to patients and conducts IND-enabling studies to initiate this process. The inventors believe that the use of large-scale, systematic genetic screens in cancer patients can identify regulators of CAR T cell function in solid tumors. Described herein, a limited set of loss-of-function (LOF) CRISPR screens are used to identify key modulators of T cell performance in human patients.

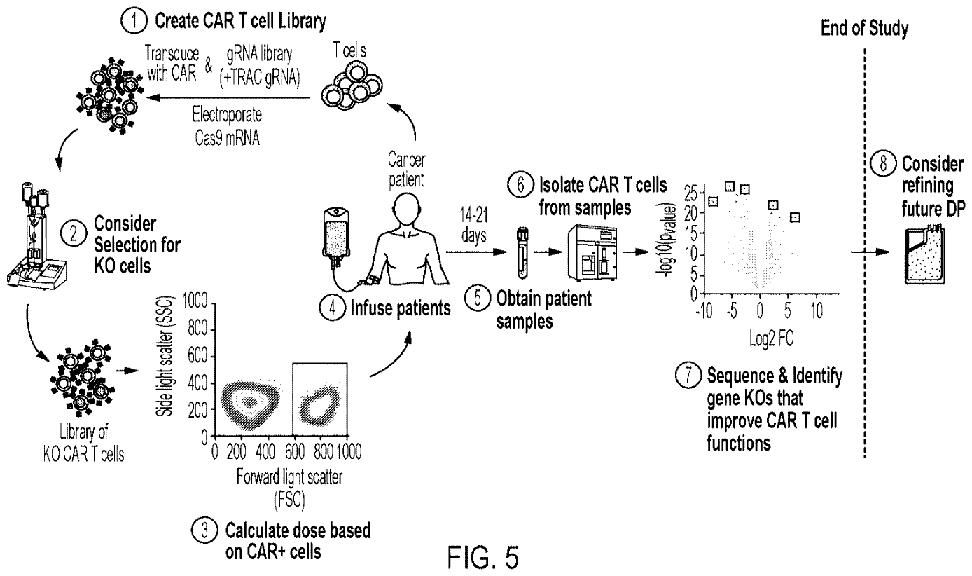

The therapeutic drug product has autologous T lymphocytes transduced with two lentiviral vectors—one encoding a CRISPR guide library (Mario Library) with sgRNAs targeting 135 genes related to T cell function and another encoding a chimeric antigen receptor (CAR) targeting mesothelin using the SS1 scFv binder (Meso-Mario-CART cells). The product manufacturing is designed so that each mesothelin-targeting CAR T cell expresses a single, variable sgRNA to a specific gene, and a single, fixed sgRNA to the TCR alpha chain gene (TRAC). The entire, limited-set library of knockout Meso-Mario-CAR T cells is delivered to each patient (FIG. 5). The diseases being treated are highly advanced, mesothelin-expressing cancers, including ovarian cancer and pancreatic cancer. The specific mesothelin CAR construct has previously been tested in Phase 1 clinical trials in these patient populations (SS1-BBz) both in the form of RNA-electroporated T cells and lentivirally-transduced T cells (6-12).

Mesothelin is highly expressed in ovarian cancer cells compared to normal cells, making it an attractive antigen to target with CAR T cells. Ovarian cancer is the fifth leading cause of cancer deaths in women in the United States (American Cancer Society, 2022). Ninety percent of ovarian cancers are epithelial, the most common being serous carcinoma. Most serous carcinomas are diagnosed at stage III (51%) or IV (29%), for which the 5-year cause-specific survival is 42% and 26%, respectively (13). Current treatment options combine debulking surgery, drug treatment, and radiation therapy. However, 70% of ovarian cancer patients relapse after primary cytoreductive surgery and standard first-line chemotherapy (14). The median survival in patients with platinum-resistant or refractory high-grade epithelial ovarian cancer is between 9-12 months (15). Such poor prognosis renders this indication an unmet medical need. Mesothelin is expressed in 75% of high-grade serous ovarian cancers making it a suitable target for immunotherapy (16).

Among other malignancies that highly express mesothelin is pancreatic adenocarcinoma (16). Pancreatic cancer is one of the deadliest solid tumor malignancies. The five-year survival rate is 11%, with around 50 thousand deaths expected in 2022 (17). Only 10-15% of the patients present with resectable disease at the time of diagnosis (18). For patients with locally-advanced and unresectable diseases, the treatment regimen usually consists of multiagent chemotherapy, from which 80% of the patients do not elicit a sufficient response to qualify for surgery (18). Finally, the treatment of metastatic pancreatic cancer consists of cytotoxic regimens with a median overall response between 6 to 11 months.

Previous trials of mesothelin-directed CAR T cells using the SS1-derived single-chain variable fragment as the binding domain of the CAR have demonstrated safety but an overall lack of clinically meaningful efficacy in patients with advanced ovarian or pancreatic cancer (6-12). In part, this is thought to be due to poor persistence of CAR T cells, and development of T cell dysfunction in the tumor microenvironment. There are hundreds of candidate genes and pathways that can modify T cell biology; it is not clear that it will ever be possible to conduct single first-in-human studies or randomized studies to test modifications of each of these candidate pathways in separate clinical trials or drug-development programs. When the therapeutic product is a gene-engineered cell, it is possible to use the gene-marking itself to test pools of product with different genetic modifications, and then identify the highest engrafting cell product after infusion. In this example, each patient provides information as to which gene-modified CAR T cell confers the highest benefit for that individual. It can also be possible to identify the highest-engrafting and/or most persistent CAR T products across patients and across two diseases that express the same target antigen (in this case, mesothelin).

A CRISPR screen is based on using a lentiviral library of guide RNAs combined with transient expression of Cas9 nuclease to generate a pool of cells that each have a single gene from the library knocked out. Thus, a CRISPR screen in human patients can be used to rapidly identify which gene knockouts from a selected and curated library of genes that are involved in modifying T cell biology, results in increased engraftment, persistence and responses in patients with advanced solid tumors that express mesothelin. The inventors believe that Meso-Mario CAR T cells offer the chance of prospective benefit to individual patients because the therapeutic product takes “multiple shots on goal,” generating CAR T cells that may have improved effector function and yet have a defined specificity (based on the SS1-BBz CAR construct) that has previously been shown to be safe (6,7). Beyond the prospective benefit for each individual human research subject on this study, using this approach, a single gene knockout may be rapidly identified that confers greater engraftment, persistence, and response across patients or across two diseases; if this is the case, then a more typical drug development pathway onward, incorporating that specific, single-gene-knockout in the context of Meso-CAR-T, can be undertaken separately, to maximize benefit for the greatest number of patients.

### Product Description and Design

The therapeutic drug product is autologous T lymphocytes transduced with two lentiviral vectors—one encoding a CRISPR sgRNA library (Mario library) targeting 135 genes related to T cell function (Table 1) as well as the TCR alpha chain (TRAC) to remove the endogenous TCR, and another lentiviral vector encoding a chimeric antigen receptor (CAR) targeting mesothelin (Meso-Mario-CART cells).

ADORA2A
AGO1
AGPS
ARID1A
ARID2
ARIH2
ATF6
BATF3
BCL6
BTLA
CABP4
CBLB
CD160
CD2
CD244
CD5
CD69
CDKN1B
CPT1B
CRELD1
CTBS
CTLA4
CXCR3
CYB5R4
DGKA
DGKZ
DLAT
DNMT1
DNMT3A
DNMT3B
DUSP4
EED
ELOB
ENTPD1
EOMES
EPAS1
ERG
ETS1
EZH2
FIBP
FLI1
FLT1
FOXP3
FUBP1
GATA3
GGH
GLRX
GNA13
HAVCR2
HIF1A
ID2
IFNAR1
IFNAR2
IFNG
IFNGR1
IFNGR2
IKZF1
IKZF2
IL10RA
IL10RB
IL13
IL18R1
IL1A
IL2RA
IREB2
IRF2
IRF4
ITK
JUNB
KDM1A
KDR
KLRB1
KLRC1
KLRD1
LAG3
LCP2
LEF1
LTA
MEF2D
MOCS3
NDUFB10
NFATC1
NFATC2
NR4A1
NR4A2
NR4A3
NT5E
PBRM1
PCBP2
PDCD1
PDCL
PDHA1
PDHB
PEX13
PRDM1
PRDM15
PTPN2
RARA
RARB
RARG
RASA2
RC3H1
RCOR1
RNF145
RPRD1B
RUNX1
RUNX3
SETDB1
SFRP1
SMAD2
SMARCA4
SMARCB1
SOCS1
STAT3
STAT6
TASOR
TBL1XR1
TCF7
TET2
TGFBR1
TGFBR2
TIGIT
TMEM222
TNFAIP3
TNFAIP8
TNFRSF18
TNFRSF9
TNIP1
TOX
UBASH3A
VAV1
VHL
VTI1A
ZC3H12A
ZFP36L1
Intergenic controls (N = 100)

### Example 6 Table 1: Genes Included in the sgRNA Library (Mario Library) of the Meso-Mario-CART Cell Product

Design of the CRISPR sgRNA Library

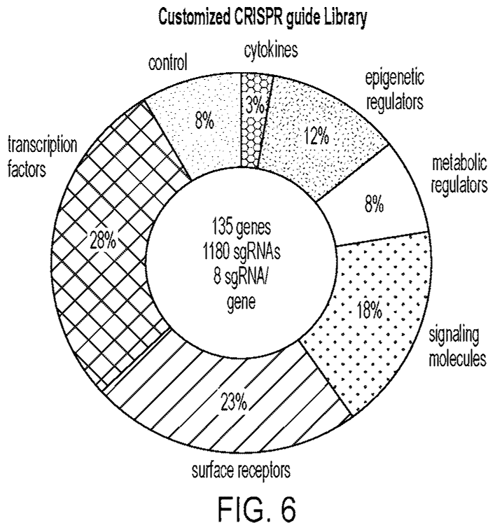

The CRISPR library was designed to maximize safety, prospect of benefit, and learnings to the field by optimal deconvolution of data obtained from each treated subject. 135 genes were selected based on a literature search and genes that were tested (i.e., IFNG, ITK, PDCD1, HAVCR2) (FIG. 6). The dominant transcripts for each gene were then chosen, and 34,318 guides were designed using CRISPick (30). To minimize potential off-target effects of sgRNA guides, guides with predicted off-target cuts in tumor suppressor genes were then removed (31). Guide RNAs targeting regions with high variability in the human population were removed. The Cutting Frequency Determination (CFD) score was used to mitigate the risk of off-target activity of any given guide (30). Guides with aggregated CFD off-target scores ≤0.27 were removed. For each gene, the Rule Set 3 score was used, which incorporates multiple sequence features that contribute to the sgRNA expression and generation of on-target gene editing to choose 8 sgRNAs per gene (32). Finally, 100 validated intergenic control guides were included for a total of 1180 guides in the library. When combined with Cas9 protein (which will be delivered in the form of mRNA via electroporation), each cell transduced with the Mario library is expected to have two genes knocked out: the TCR alpha chain, and a single gene from the library.

### Design of Lentiviral Vectors

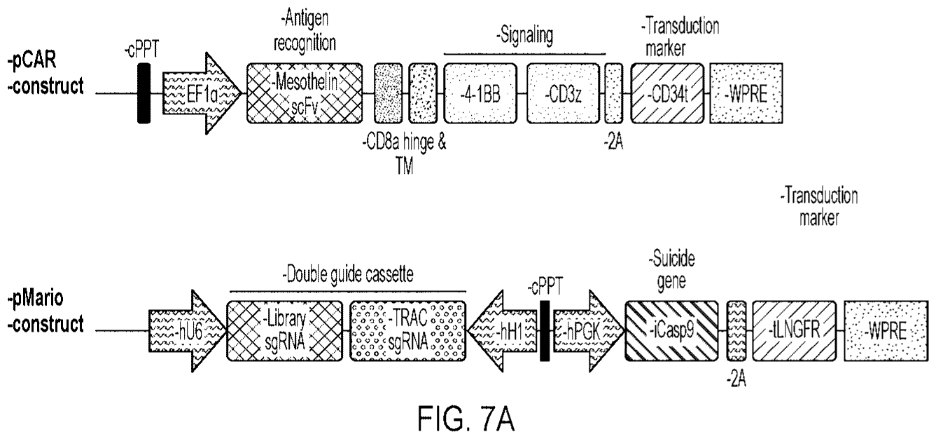

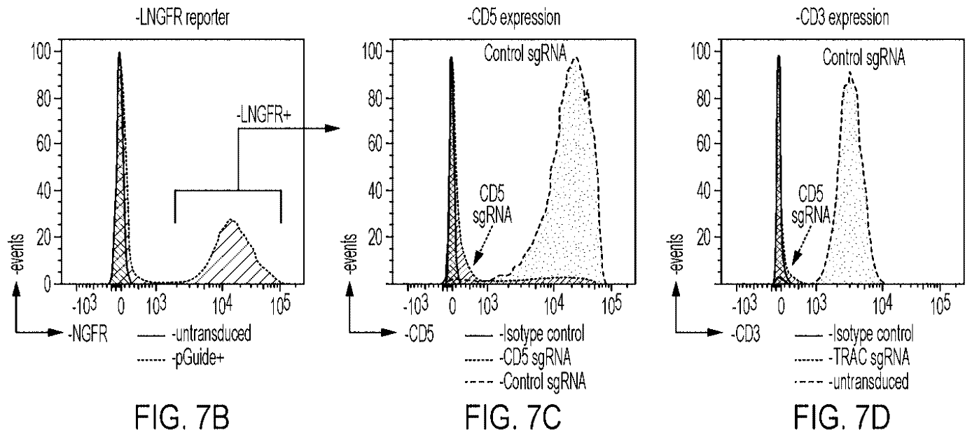

Two lentiviral vector constructs were designed for safe delivery of the CRISPR sgRNA library (pMario) and a chimeric antigen receptor targeting mesothelin (pCAR, FIG. 7A). For the construct containing the CRISPR library (pMario), another layer of safety was added by incorporating an inducible caspase 9 (iCasp9)-based safety switch that could eliminate CAR T cells in case of severe adverse events or outgrowth of a CAR T cell clone. To optimize expression of two sgRNAs, a dual sgRNA cassette was designed where the sgRNA library was cloned into position 1, and position 2 contains an sgRNA to the T cell receptor alpha constant gene (TRAC). Because assembly of the natural T cell receptor/CD3 complex requires an intact TCR alpha chain, knockout of TRAC results in loss of expression of the entire native TCR/CD3 complex. Thus, the TRAC-targeting guide allows for the negative selection of edited cells during manufacturing (using CliniMACs to perform CD3-based magnetic selection of the investigational product). Clinically, this selection for CD3-negative T cells also minimizes the risk of potentiating a self-reactive T cell (based on a retained TCR of undefined specificity). Finally, a truncated low-affinity nerve growth factor receptor ((LNGFR) reporter was included to allow for flow-cytometry-based enumeration, recovery, and enrichment of the cells after infusion, and facilitates further analysis of the enriched and depleted sgRNAs. The pCAR vector (FIG. 7A) was designed to contain a CAR construct consisting of the SS1 scFv that binds mesothelin and a 4-1BB intracellular co-stimulatory domain (6-11). The construct also contains a CD34t reporter to allow for dosing of CAR+ T cells and post-infusion monitoring.

Validation of the sgRNA Double Cassette and the Reporter System

To determine the reporter expression and editing efficiency of the dual sgRNA cassette, bead-activated T cells (CD3/CD28 beads at 3:1 beads:T cell ratio) were transduced with a double guide vector that encoded sgRNAs targeting CD5 and TRAC. After de-beading, the cells were electroporated with Cas9 mRNA. Naïve and transduced T cells were then analyzed by flow cytometry for cell surface expression of tLNGFR, CD3, and CD5 (FIG. 7B-7D). Isotype controls, which are similar fluorescent antibody that lacks specificity to the analyzed target, were used as a negative control. Results show that LNGFR was expressed in the double guide vector transduced T cells (FIG. 7B). Untransduced, naïve T cells maintain expression of the TCR (CD3); however, double guide vector transduced cells lack CD3 expression, resulting from successful targeting of TRAC with the sgRNA following Cas9 electroporation (FIG. 7C). LNFGR+ cells also lack CD5 expression, demonstrating the appropriate function of the double guide cassette vector.

### Generation of Meso-Mario-CART Cell for In Vivo Testing

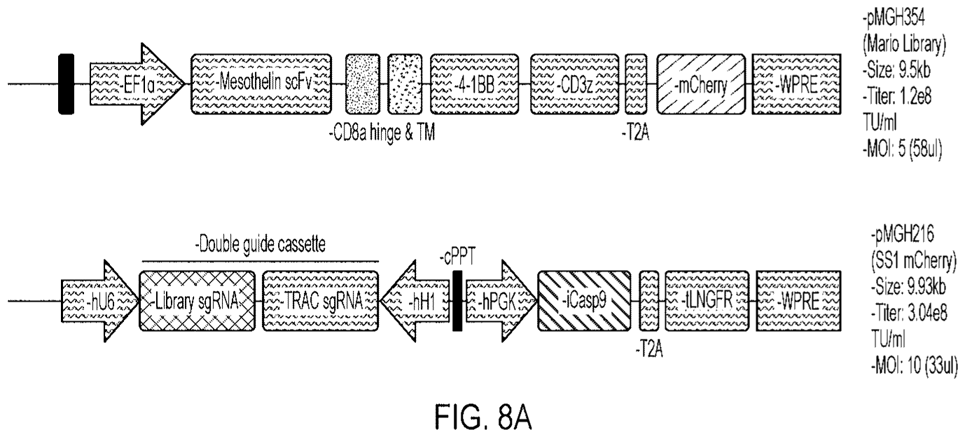

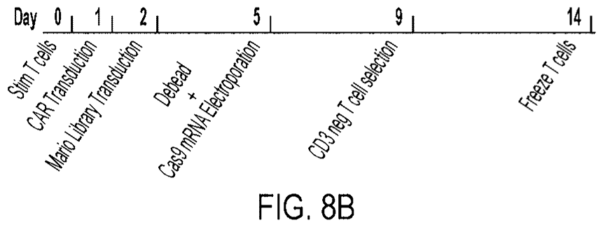

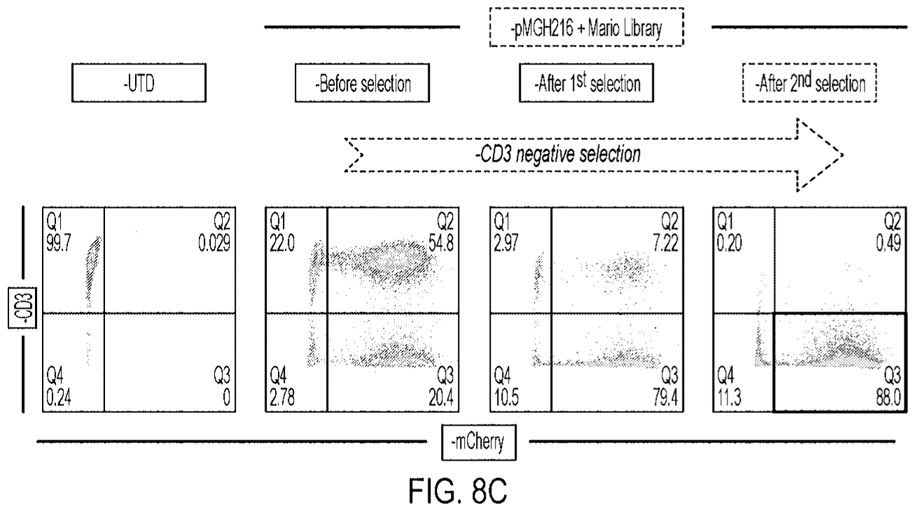

A protocol for production of Meso-Mario-CART cells for in vivo testing was determined. On day 0, T cells were activated using CD3/CD28 beads. The cells were then transduced with lentivirus harboring the anti-mesothelin CAR. On day 2, the cells were transduced with the lentiviral vector containing the CRISPR (Mario) library. The cells were then transfected with Cas9 mRNA via electroporation on day 5. The cells were expanded to day 10, and CD3-negative cells were enriched by depleting CD3-positive cells using an APC-CD3 selection kit. After two rounds of selection, the protocol was able to generate final products with up to 88% double transduced cells (mCherry+CD3−) (FIGS. 8A-8C).

### In Vivo Pilot Study of the CRISPR Screen

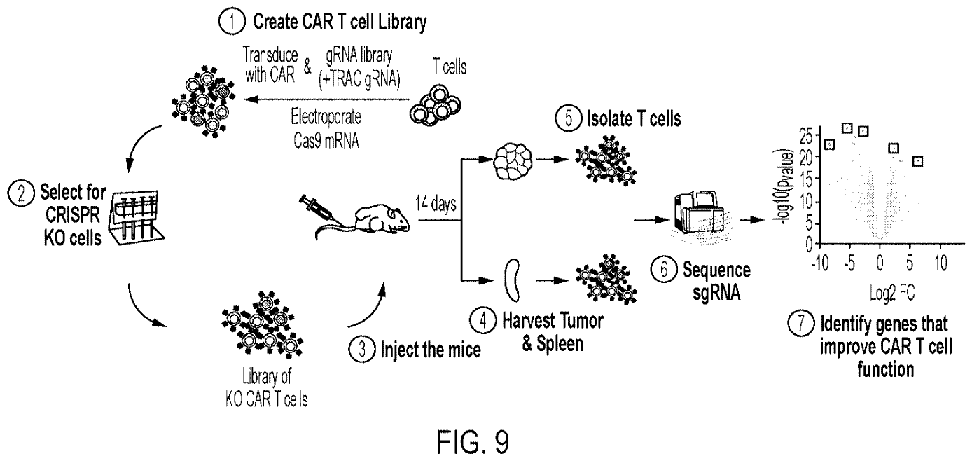

In preparation for human CRISPR screens, pilot CRISPR screens in vivo in NSG mice were performed (FIG. 9). The described Mario sgRNA library targeting the selected 135 relevant T cell regulators was generated, with 8 sgRNAs per gene. T cells were transduced with this library and mesothelin CAR, and electroporated T cells with Cas9 mRNA as described. The library-transduced T cells were transferred into mesothelin+ ASPC1 tumor-bearing mice. After 14 days the CAR T cells were isolated from the tumor after digestion and positive selection for human CD45. Data were generated by PCR amplification of sgRNAs from gDNA of isolated T cells, followed by next-generation sequencing (NGS).

The recovered CAR T cells from the tumors and spleens of ASPC1 tumor-bearing mice demonstrate that an ability to engraft a high number of tumor-infiltrating mesothelin CAR T cells and efficiently capture the T cells from the tumor and spleen, allowing for a technically robust in vivo screening experiment even in small numbers of animals (FIG. 10A). The distribution of the sgRNAs in the recovered CAR T cells indicates that we can capture the full CRISPR library, and the guides are well represented in the recovered pools of cells.

These results identified that the top hits during an in vivo screen, were in fact the opposite of what the top predicted hits from a published whole-genome CRISPR screen that was conducted in vitro (5). In these results, knockout of IL2RA was enriched in vivo, whereas knockout of RASA2 was depleted (FIG. 10B). In previous in vitro screens RASA2 was among the most enriched gene knockouts in T cells. Furthermore, these data illustrate that using in vitro models to predict the best genetic knockout targets to enhance CAR T cell function may not in fact predict in vivo function or activity. Similar results were observed using a BCMA CAR indicating that the effects of the gene knockouts are not CAR-specific (see Example 8).

Production of Large-Scale sgRNA Plasmid Library

Based on our pre-clinical data results, the feasibility of producing the plasmid library (Mario library) at a large scale and with investigational new drug (IND)-Ready methodology with sufficient quality to enable current good manufacturing practice (cGMP)-grade lentiviral vector manufacturing was assessed. To assess the quality of the large-scale guide library, the guide distribution of the original plasmid library stock was compared to the large-scale IND-Ready plasmid preparation. In the large scale preparation, the guide abundance in the plasmids was first analyzed using NGS of PCR products flanking the guide region. Then the primary human T cells were transduced with lentivirus prepared side-by-side using either the original plasmid primary library stock or the large-scale plasmid preparation.

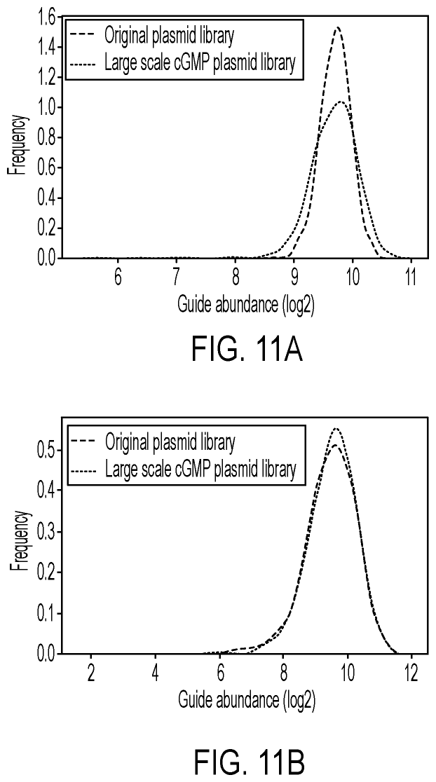

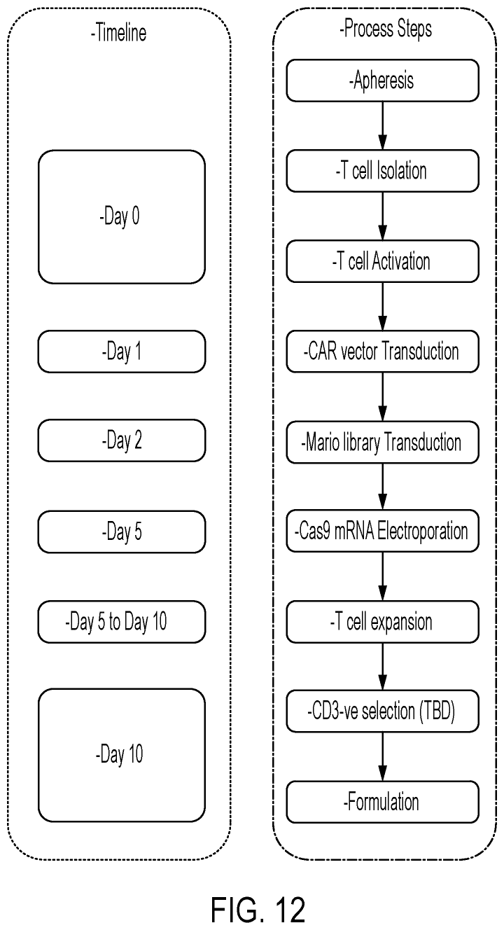

Results indicate that the large-scale plasmid preparation process did not significantly alter the distribution of plasmids in the library (FIG. 11A). Furthermore, the distribution of guides in the transduced T cells was not significantly altered (FIG. 11B), indicating that the plasmid preparation can be used to make lentivirus and cell products suitable for clinical-grade screening studies. FIG. 12 outlines a cGMP-compliant manufacturing process for the Meso-Mario-CAR T cell product (FIG. 12).

### Treatment Plan

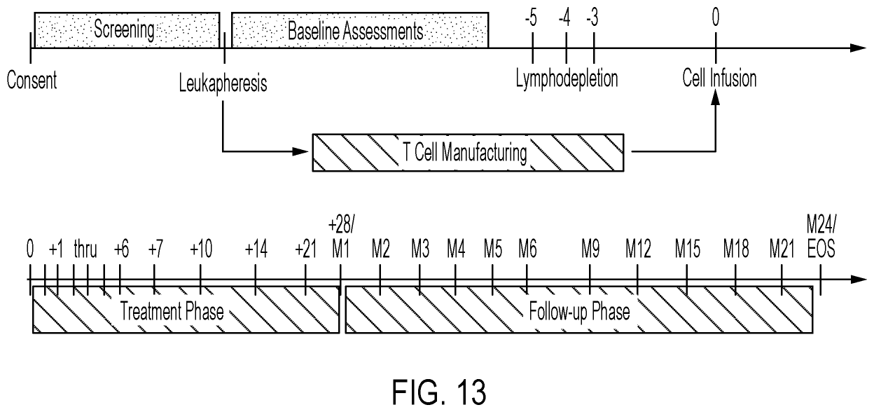

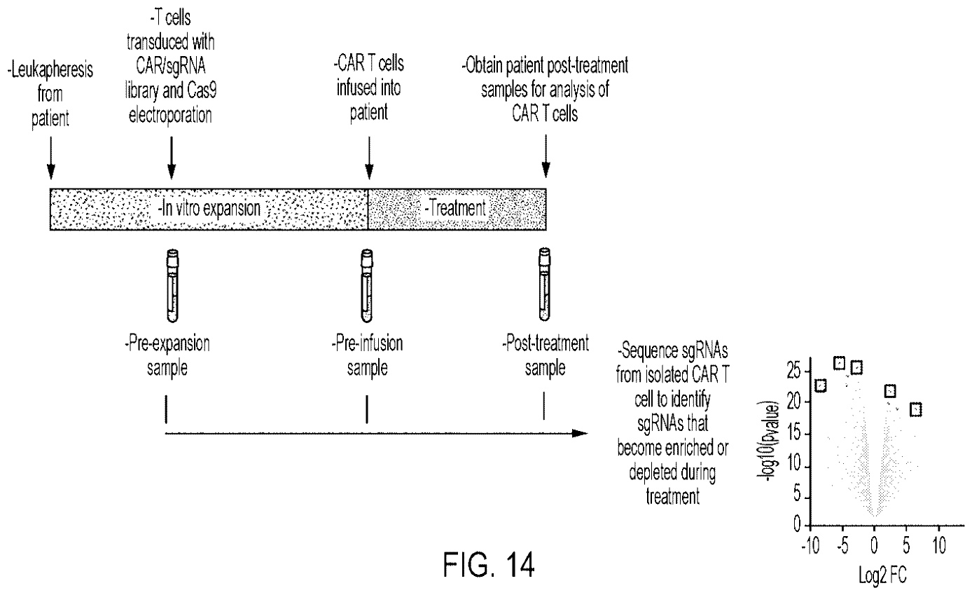

Human patients may undergo 3 days of lymphodepletion chemotherapy starting about 5 days before the infusion of Meso-Mario-CART cells on Day 0 (FIG. 13). Lymphodepletion can be given outpatient or inpatient, per the medical provider's judgment. Following CAR T cell infusion, patients are monitored for adverse events, clinical status, and laboratory parameters. Research samples are collected for correlative studies at the time points indicated (FIG. 13 and FIG. 14) for up to 24 months. Research samples are analyzed using the methods described herein (e.g., in Examples 4-6).

### REFERENCES

1. Larson R C, Kann M C, Bailey S R, Haradhvala N J, Llopis P M, Bouffard A A, et al. CAR T cell killing requires the IFNγR pathway in solid but not liquid tumours. Nature [Internet]. 2022 Apr. 21 [cited 2023 Mar. 2]; 604 (7906):563-70. Available from: https://www.nature.com/articles/s41586-022-04585-5
2. Bailey S R, Vatsa S, Larson R C, Bouffard A A, Scarfò I, Kann M C, et al. Blockade or Deletion of IFNγ Reduces Macrophage Activation without Compromising CAR T cell Function in Hematologic Malignancies. Blood Cancer Discov [Internet]. 2022 Mar. 1 [cited 2023 Mar. 2]; 3 (2):136-53. Available from: https://aacrjournals.org/bloodcancerdiscov/article/3/2/136/681793/Blockade-or-Deletion-of-IFN-Reduces-Macrophage
3. Lin S, Larrue C, Scheidegger N K, Seong B K A, Dharia N V, Kuljanin M, et al. An In Vivo CRISPR Screening Platform for Prioritizing Therapeutic Targets in AML. Cancer Discov [Internet]. 2022 Feb. 1 [cited 2023 Mar. 2]; 12 (2):432-49. Available from: https://aacrjournals.org/cancerdiscovery/article/12/2/432/678482/An-In-Vivo-CRISPR-Screening-Platform-for
4. Michels B E, Mosa M H, Streibl B I, Zhan T, Menche C, Abou-El-Ardat K, et al. Pooled In Vitro and In Vivo CRISPR-Cas9 Screening Identifies Tumor Suppressors in Human Colon Organoids. Cell Stem Cell [Internet]. 2020 May [cited 2023 Mar. 2]; 26 (5):782-792.e7. Available from: https://linkinghub.elsevier.com/retrieve/pii/S1934590920301429
5. Carnevale J, Shifrut E, Kale N, Nyberg W A, Blaeschke F, Chen Y Y, et al. RASA2 ablation in T cells boosts antigen sensitivity and long-term function. Nature [Internet]. 2022 Sep. 1 [cited 2023 Mar. 2]; 609 (7925):174-82. Available from: https://www.nature.com/articles/s41586-022-05126-w
6. Haas A R, Tanyi J L, O'Hara M H, Gladney W L, Lacey S F, Torigian D A, et al. Phase I Study of Lentiviral-Transduced Chimeric Antigen Receptor-Modified T Cells Recognizing Mesothelin in Advanced Solid Cancers. Mol Ther [Internet]. 2019 November [cited 2023 Mar. 15]; 27 (11):1919-29. Available from: https://linkinghub.elsevier.com/retrieve/pii/S1525001619303284
7. Beatty G L, O'Hara M H, Lacey S F, Torigian D A, Nazimuddin F, Chen F, et al. Activity of Mesothelin-Specific Chimeric Antigen Receptor T Cells Against Pancreatic Carcinoma Metastases in a Phase 1 Trial. Gastroenterology [Internet]. 2018 July [cited 2023 Mar. 15]; 155 (1):29-32. Available from: https://linkinghub.elsevier.com/retrieve/pii/S0016508518303238
8. Tanyi J L, Stashwick C, Plesa G, Morgan M A, Porter D, Maus M V, et al. Possible Compartmental Cytokine Release Syndrome in a Patient With Recurrent Ovarian Cancer After Treatment With Mesothelin-targeted CAR-T Cells. J Immunother [Internet]. 2017 April [cited 2023 Apr. 3]; 40 (3):104-7. Available from: https://journals.lww.com/00002371-201704000-00003
9. Ko A H, Jordan A C, Tooker E, Lacey S F, Chang R B, Li Y, et al. Dual Targeting of Mesothelin and CD19 with Chimeric Antigen Receptor-Modified T Cells in Patients with Metastatic Pancreatic Cancer. Mol Ther [Internet]. 2020 November [cited 2023 Apr. 3]; 28 (11):2367-78. Available from: https://linkinghub.elsevier.com/retrieve/pii/S1525001620303683
10. Maus M V, Haas A R, Beatty G L, Albelda S M, Levine B L, Liu X, et al. T Cells Expressing Chimeric Antigen Receptors Can Cause Anaphylaxis in Humans. Cancer Immunol Res [Internet]. 2013 Jul. 1 [cited 2023 Apr. 3]; 1 (1):26-31. Available from: https://aacrjournals.org/cancerimmunolres/article/1/1/26/466725/T-Cells-Expressing-Chimeric-Antigen-Receptors-Can
11. Beatty G L, Haas A R, Maus M V, Torigian D A, Soulen M C, Plesa G, et al. Mesothelin-Specific Chimeric Antigen Receptor mRNA-Engineered T Cells Induce Antitumor Activity in Solid Malignancies. Cancer Immunol Res [Internet]. 2014 Feb. 1 [cited 2023 Apr. 3]; 2 (2):112-20. Available from: https://aacrjournals.org/cancerimmunolres/article/2/2/112/467025/Mesothelin-Specific-Chimeric-Antigen-Receptor-mRNA
12. Adusumilli P S, Zauderer M G, Rivière I, Solomon S B, Rusch V W, O'Cearbhaill R E, et al. A Phase I Trial of Regional Mesothelin-Targeted CAR T-cell Therapy in Patients with Malignant Pleural Disease, in Combination with the Anti-PD-1 Agent Pembrolizumab. Cancer Discov [Internet]. 2021 Nov. 1 [cited 2023 Apr. 4]; 11 (11):2748-63. Available from: https://aacrjournals.org/cancerdiscovery/article/11/11/2748/666400/A-Phase-I-Trial-of-Regional-Mesothelin-Targeted
13. Torre L A, Trabert B, DeSantis C E, Miller K D, Samimi G, Runowicz C D, et al. Ovarian cancer statistics, 2018: Ovarian Cancer Statistics, 2018. CA Cancer J Clin [Internet]. 2018 July [cited 2023 Mar. 2]; 68 (4):284-96. Available from: http://doi.wiley.com/10.3322/caac.21456
14. Giornelli G H. Management of relapsed ovarian cancer: a review. SpringerPlus [Internet]. 2016 December [cited 2023 Mar. 2]; 5 (1):1197. Available from: http://springerplus.springeropen.com/articles/10.1186/s40064-016-2660-0
15. Davis A, Tinker A V, Friedlander M. “Platinum resistant” ovarian cancer: What is it, who to treat and how to measure benefit? Gynecol Oncol [Internet]. 2014 June [cited 2023 Mar. 2]; 133 (3):624-31. Available from: https://linkinghub.elsevier.com/retrieve/pii/S0090825814002029
16. Hassan R, Thomas A, Alewine C, Le D T, Jaffee E M, Pastan I. Mesothelin Immunotherapy for Cancer: Ready for Prime Time? J Clin Oncol [Internet]. 2016 Dec. 1 [cited 2023 Mar. 2]; 34 (34):4171-9. Available from: https://ascopubs.org/doi/10.1200/JCO.2016.68.3672
17. Jiang Y, Sohal D P S. Pancreatic Adenocarcinoma Management. JCO Oncol Pract [Internet]. 2023 January [cited 2023 Mar. 2]; 19 (1):19-32. Available from: https://ascopubs.org/doi/10.1200/OP.22.00328
18. Park W, Chawla A, O'Reilly E M. Pancreatic Cancer: A Review. JAMA [Internet]. 2021 Sep. 7 [cited 2023 Mar. 2]; 326 (9):851. Available from: https://jamanetwork.com/journals/jama/fullarticle/2783817
30. Doench J G, Fusi N, Sullender M, Hegde M, Vaimberg E W, Donovan K F, et al. Optimized sgRNA design to maximize activity and minimize off-target effects of CRISPR-Cas9. Nat Biotechnol [Internet]. 2016 February [cited 2023 Mar. 2]; 34 (2):184-91. Available from: http://www.nature.com/articles/nbt.3437
31. Lenoir W F, Morgado M, DeWeirdt P C, Mclaughlin M, Griffith A L, Sangree A K, et al. Discovery of putative tumor suppressors from CRISPR screens reveals rewired lipid metabolism in acute myeloid leukemia cells. Nat Commun [Internet]. 2021 Nov. 11 [cited 2023 Mar. 2]; 12 (1):6506. Available from: https://www.nature.com/articles/s41467-021-26867-8
32. DeWeirdt P C, McGee A V, Zheng F, Nwolah I, Hegde M, Doench J G. Accounting for small variations in the tracrRNA sequence improves sgRNA activity predictions for CRISPR screening. Nat Commun [Internet]. 2022 Sep. 6 [cited 2023 Mar. 2]; 13 (1):5255. Available from: https://www.nature.com/articles/s41467-022-33024-2

### Example 8: CAR-T Cell CRISPR Library in Liquid Tumors

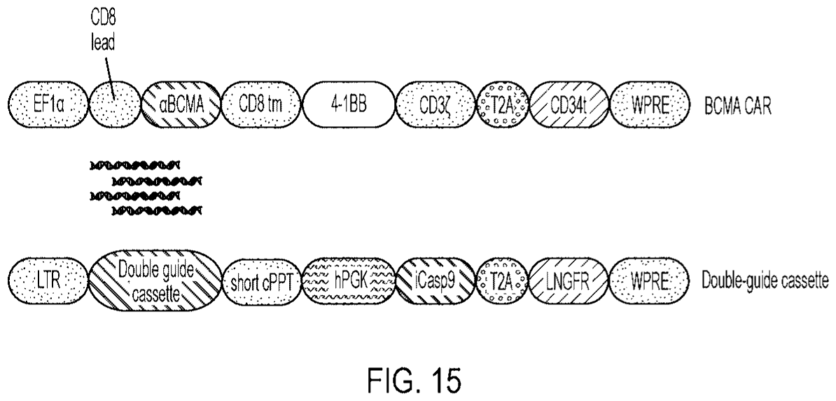

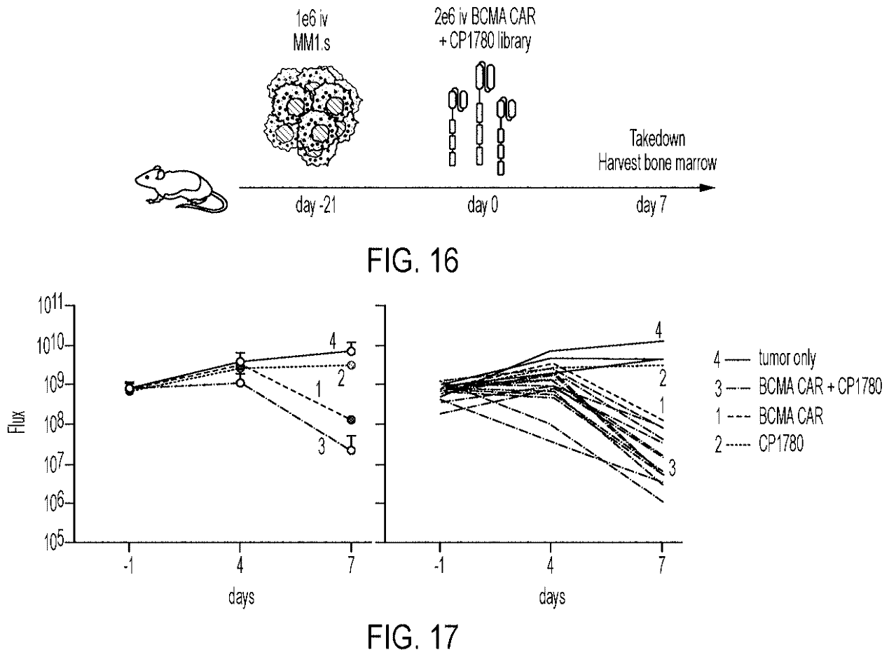

CAR-T cell therapies have proven effective in treating some hematologic cancers. However, challenges still remain in treating hematological cancers like multiple myeloma. For example, BCMA CAR-T cells have shown a initial response against multiple myeloma, but most patients eventually relapse. Thus, there is a need for improving CAR-T cell therapy for treating multiple myeloma. To address this, the Mario CRISPR library described in Example 7 (also referred to as +CP1789 library) was applied to BCMA CAR-T cells to produce a BCMA CAR-T cell+Mario library (see FIG. 15 for construct illustrations). Each of the genes selected for targeting in the Mario library were related to CAR-T cell function and thus expected to influence CAR-T cell function. The BCMA CAR-T cell+Mario library was administered to mice having a human multiple myeloma cancer (FIG. 17, top panel). Results show that the BCMA CAR-T cell+Mario library was capable of killing cancer cells (FIG. 17, bottom panel).

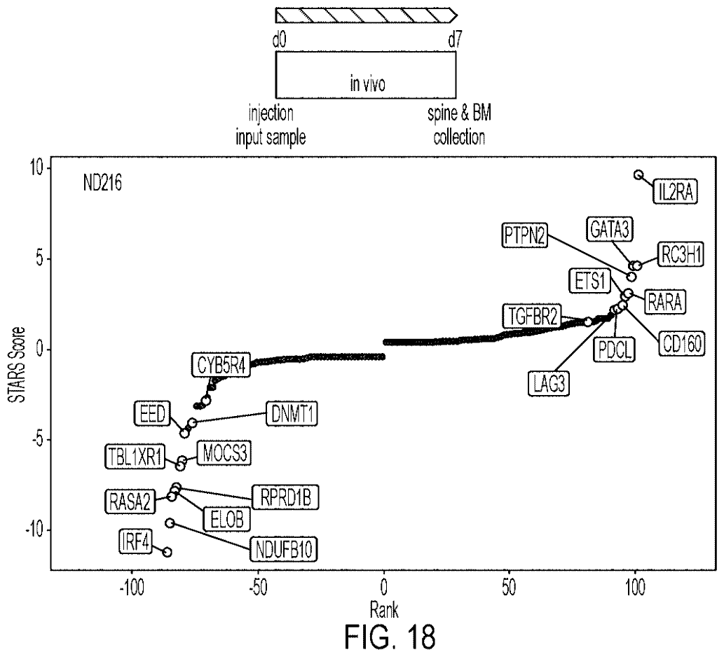

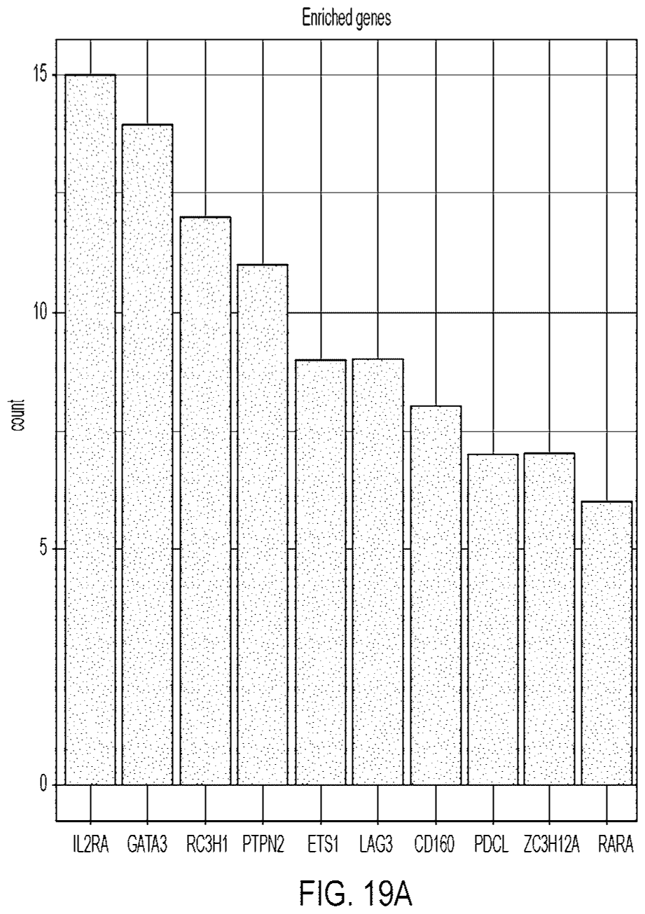

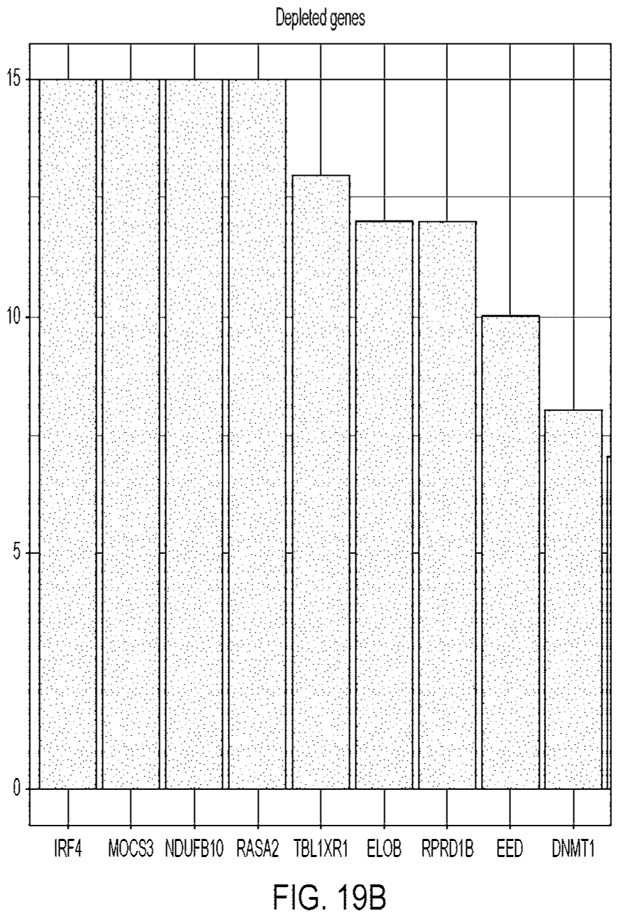

To measure individual BCMA CAR-T Cell CRISPR targeting effects in vivo, CAR-T cells were sampled from the library and gRNA polynucleotide were sequenced, which is indicative of the relative abundance of a CAR-T cell comprising a knockout mutation of the gene targeted by the gRNA polynucleotide. This sequencing data was analyzed to determine which gene knockouts increased or decreased CAR-T relative abundance over a given time. Initial experiments administered the BCMA CAR-T cell+Mario library at day 0 (21 days after cancer cell administration) and collected a sample of the BCMA CAR-T cell+Mario library from the mouse at day 7 for sequencing (FIG. 18). Results identified genes knockouts that enriched CAR-T cells abundance in vivo (e.g., IL2RA) and gene knockouts that decreased CAR-T cell abundance in vivo (e.g., RASA2) (FIGS. 19A-19B). Surprisingly, RASA2 had previously been shown to be highly enriched in vitro CAR-T cell knockout screens. Additionally, a similar results were seen for mesothelin CAR treating pancreatic cancer.

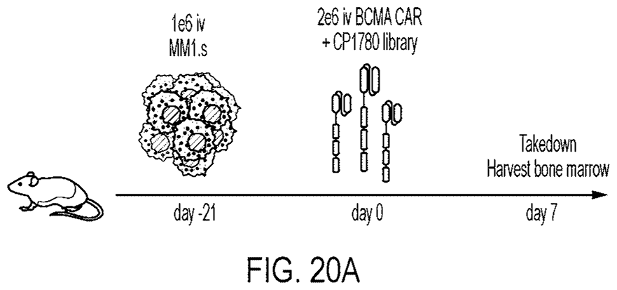

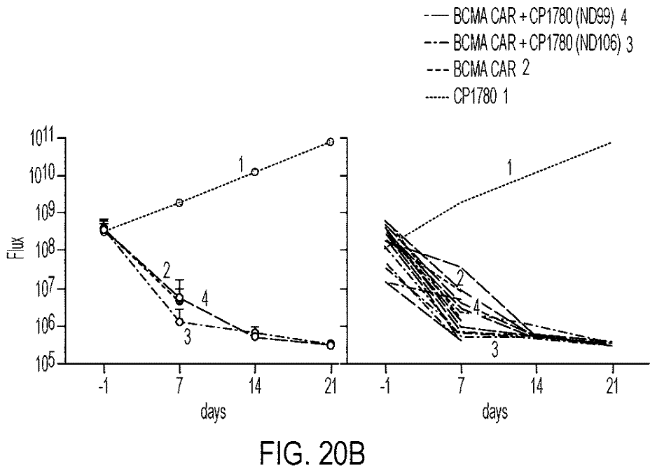

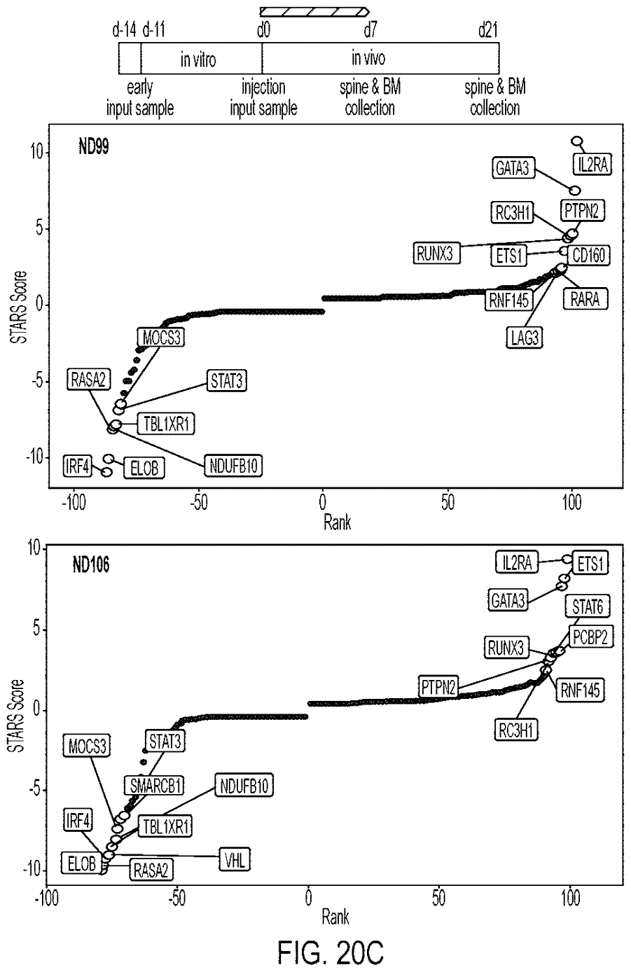

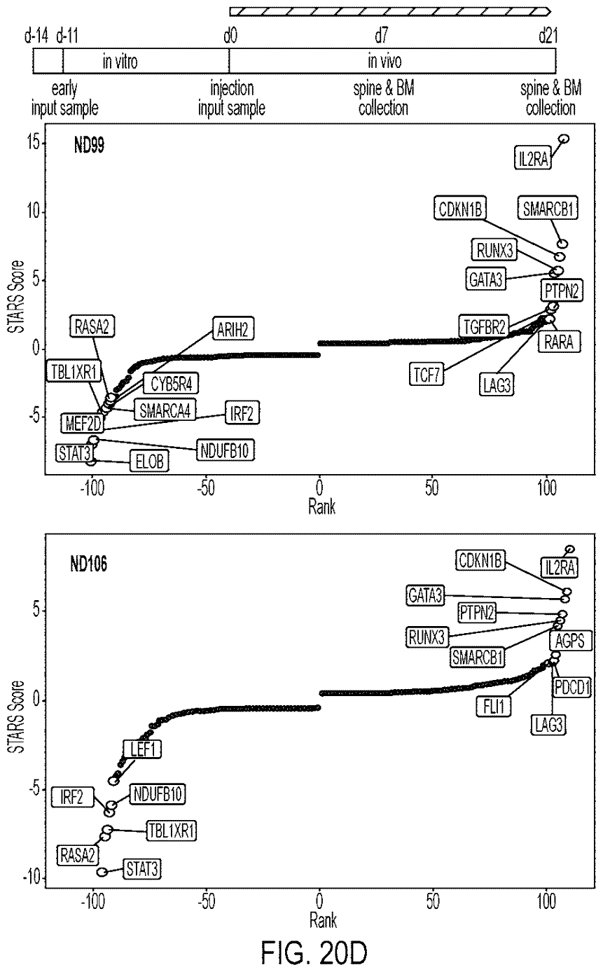

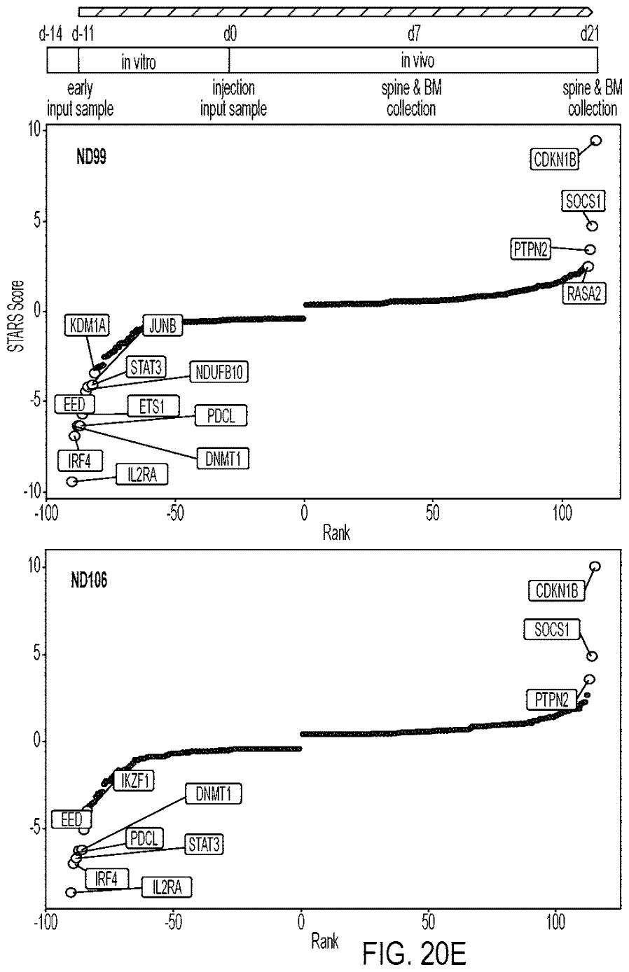

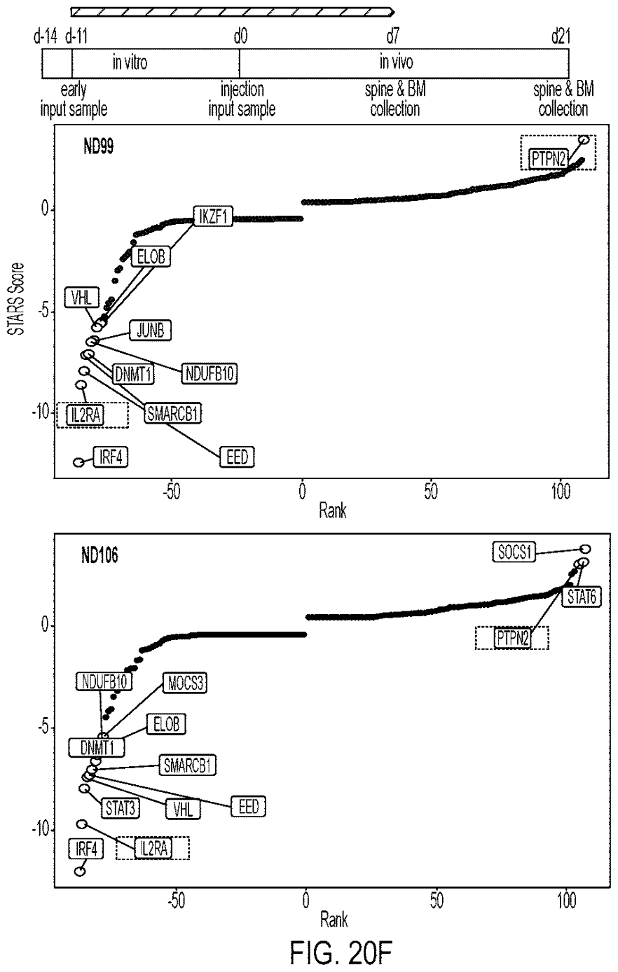

Additional experiments were performed using different T cell donors (but the same BCMA CAR and Mario library) and for different time periods to determine reproducibility and to estimate how long the BCMA CAR-T cell+Mario library should be in the subject before collecting the BCMA CAR-T cell+Mario library and sequencing (FIGS. 20A-20F)). Results indicate that administering the BCMA CAR-T cell+Mario library at day 0 (21 days after giving cancer cells to the mice) and collecting the BCMA CAR-T cell+Mario library from the subject at day 7 or day 21 results in higher resolution of CAR-T cell knockout effect than administering the BCMA CAR-T cell+Mario library at day-11 (10 days after giving cancer cells to the mice) and collecting the BCMA CAR-T cell+Mario library from the subject at day 7 (FIGS. 20C, 20D, and 20F). Additionally, results were reproducible between experimental replicates, ND99 and ND106, which demonstrates the reproducibility of the assay. Further, IL2RA was identified as the most enriched CAR-T cell gene knockout between replicates and over different CAR-T collection times as shown FIGS. 18, 20C and 20D.

### Example 9. Current Good Manufacturing Practice (cGMP)-Grade CRISPR Plasmid Library Preparation

Regulatory agencies, like the Food and Drug Administration, require cGMP compliance in producing the vectors that go into CAR-T cells for clinical use in to humans (e.g., 21 C.F.R. §§ 210, 211). However, current accepted protocols for producing cGMP compliant vectors are not applicable to producing the gRNA vector libraries (e.g., the Mario library) as described herein. Standard protocols were not applicable because these protocols are designed for production of a single plasmid (to be used in a vector), not a library of plasmids. Particularly, in cGMP standard protocols, a plasmid is transformed into chemically competent E. Coli, plated on agar plates with antibiotics, a single colony is selected from the agar plate, the colony is sequenced for quality control, and then produced at large scale. However, cGMP production of gRNA vector libraries selection of many colonies (thousands to millions) to produce a high quality gRNA vector library. Thus a new cGMP production protocol needed to be developed that maintained cGMP quality standards and the maintain the diversity of the gRNA vector library.

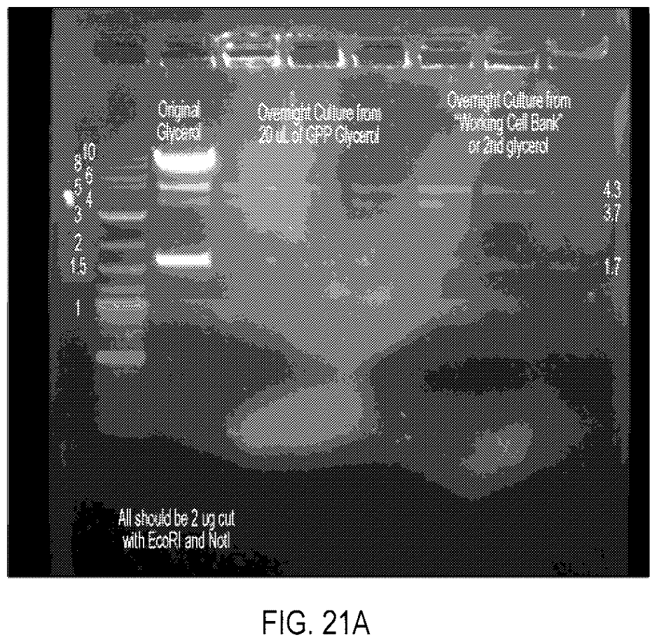

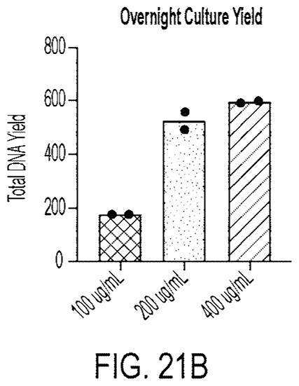

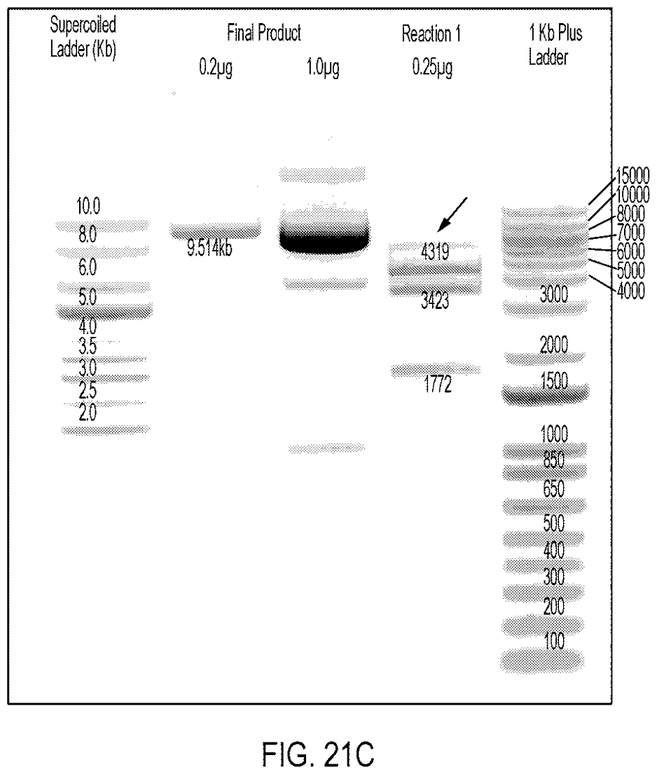

The central challenge in developing this protocol was producing a gRNA vector library of sufficient quality and diversity (e.g., coverage) of the pool of guide RNAs. To maintain library diversity, the gRNA vector library (e.g., the Mario library) was transformed into electro-competent E. coli, plated on an agar plate with antibiotics, the plate is scraped to remove the colonies that grew on the plate, then a glycerol stock is made, which was sent to a contract research organization (CRO) that specializes in investigation new drug cGMP production. The CRO proceed with cGMP production using the glycerol stock, however, the plasmid yield and quality was surprisingly low (data not shown). Similar results were observed by the inventors (FIG. 21A). Particularly, the same amount of DNA was loaded into lanes 1-7, but DNA from overnight growth of the glycerol stock showed greatly decreased yields compared to the original glycerol stock, which is evidence of low quality DNA. After attempting many different methods for solving this problem, it was discovered that increasing selection (e.g., selection for E. coli having a gRNA plasmid) by increasing the antibiotic concentration by 2-4 fold greatly improved Mario library yield and quality (FIGS. 21B-21C). Specifically, increasing Carbenicillin from 100 μg/mL to 200 μg/mL in cultures grown E. coli transformed with Mario library improved quality and yield. This protocol was then used to produce cGMP vector with the CRO, which maintained library diversity and quality (FIG. 11B).

In summary, a key to maintaining the diversity and equal distribution of plasmids in the library is the high-efficiency electrocompetent bacterial transformation of starting material and scraping of glycerol stocks which each maintain >1000 copies of each plasmid. The non-standard increased antibiotic concentration (200 ug/mL Carbenicillin) greatly improved plasmid yields and quality. Overall, this show that a cGMP, IND-ready gRNA vector library can be produced.

### Additional Sequence Tables

The following tables list sequences of exemplary genes selected for mutation in immune cells (e.g., CAR-T cells, e.g., CAR-NK cells) (Table 2), exemplary guide RNA homology regions for targeting said exemplary genes (Table 4), and exemplary CAR vector and gRNA vectors (Table 5), and exemplary CAR sequences (Table 6).

Lengthy table referenced here
US20260176622A1-20260625-T00001
Please refer to the end of the specification for access instructions.

Lengthy table referenced here
US20260176622A1-20260625-T00002
Please refer to the end of the specification for access instructions.

Lengthy table referenced here
US20260176622A1-20260625-T00003
Please refer to the end of the specification for access instructions.

Lengthy table referenced here
US20260176622A1-20260625-T00004
Please refer to the end of the specification for access instructions.

Lengthy table referenced here
US20260176622A1-20260625-T00005
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260176622A1). An electronic copy of the table will also be available from the USPTO upon request and payment of the fee set forth in 37 CFR 1.19(b)(3).

1. A composition comprising a plurality of guide RNA (gRNA) polynucleotides, wherein at least 2 gRNA polynucleotides each comprise a homology region that is complementary to a sense strand or an antisense strand of a different gene encoded by a sequence of any one of SEQ ID NOs: 1-135 or a variant thereof.

2.-13. (canceled)

14. The composition of claim 1, comprising at least 8 gRNA polynucleotides per gene sequence, wherein the at least 8 gRNA polynucleotides per gene sequence comprises different sequences.

15. The composition of claim 14, wherein the or at least 8 gRNA polynucleotides per gene sequence are complementary to non-overlapping regions of the same gene sequence.

16.-19. (canceled)

20. The composition of claim 1, the composition comprising at least one gRNA polynucleotide comprising a sequence of any one of SEQ ID NOS: 136-1315.

21. The composition of claim 1, the composition comprising at least 10 gRNA polynucleotides each comprising a different sequence of any one of SEQ ID NOs: 136-1315.

22.-23. (canceled)

24. A composition, comprising a plurality of gRNA polynucleotides comprising the sequences of SEQ ID NOs: 136-1315.

25.-30. (canceled)

31. The composition of claim 1, wherein the plurality of guide RNA polynucleotides consists of at most 5,000 gRNA polynucleotides having different sequences.

32.-54. (canceled)

55. The composition of claim 1, wherein at least 1 gRNA polynucleotide comprises a homology region that is complementary to a sense strand or an antisense strand of a different gene encoded by any one of IL2RA (SEQ ID NO: 65) or a variant thereof, GATA3 (SEQ ID NO: 46) or a variant thereof, AGPS (SEQ ID NO: 3) or a variant thereof, PTPN2 (SEQ ID NO: 98) or a variant thereof, LAG3 (SEQ ID NO: 76) or a variant thereof, PDCD1 (SEQ ID NO: 91) or a variant thereof, TGFBR2 (SEQ ID NO: 121) or a variant thereof, RARA (SEQ ID NO: 99) or a variant thereof, SmarcB1 (SEQ ID NO: 113) or a variant thereof, CDKN1B (SEQ ID NO: 18) or a variant thereof, RunX (SEQ ID NO: 107) or a variant thereof, and TCF7 (SEQ ID NO: 118) or a variant thereof.

56.-59. (canceled)

60. The composition of claim 1, comprising:
a first gRNA polynucleotide comprising a homology region that is complementary to a sense strand or an antisense strand of a gene encoded by IL2RA (SEQ ID NO: 65) or a variant thereof;
a second gRNA polynucleotide comprising a homology region that is complementary to a sense strand or an antisense strand of a gene encoded by GATA3 (SEQ ID NO: 46) or a variant thereof; a third gRNA polynucleotide comprising a homology region that is complementary to a sense strand or an antisense strand of a gene encoded by AGPS (SEQ ID NO: 3) or a variant thereof;
a fourth gRNA polynucleotide comprising a homology region that is complementary to a sense strand or an antisense strand of a gene encoded by PTPN2 (SEQ ID NO: 98) or a variant thereof;
a fifth gRNA polynucleotide comprising a homology region that is complementary to a sense strand or an antisense strand of a gene encoded by LAG3 (SEQ ID NO: 76) or a variant thereof;
a sixth gRNA polynucleotide comprising a homology region that is complementary to a sense strand or an antisense strand of a gene encoded by PDCD1 (SEQ ID NO: 91) or a variant thereof;
a seventh gRNA polynucleotide comprising a homology region that is complementary to a sense strand or an antisense strand of a gene encoded by TGFBR2 (SEQ ID NO: 121) or a variant thereof;
a eighth gRNA polynucleotide comprising a homology region that is complementary to a sense strand or an antisense strand of a gene encoded by RARA (SEQ ID NO: 99) or a variant thereof;
a nineth gRNA polynucleotide comprising a homology region that is complementary to a sense strand or an antisense strand of a gene encoded by SmarcB1 (SEQ ID NO: 113) or a variant thereof;
a tenth gRNA polynucleotide comprising a homology region that is complementary to a sense strand or an antisense strand of a gene encoded by CDKN1B (SEQ ID NO: 18) or a variant thereof or a variant thereof;
a eleventh gRNA polynucleotide comprising a homology region that is complementary to a sense strand or an antisense strand of a gene encoded by RunX (SEQ ID NO: 107) or a variant thereof;
and/or
a twelve gRNA polynucleotide comprising a homology region that is complementary to a sense strand or an antisense strand of a gene encoded by TCF7 (SEQ ID NO: 118) or a variant thereof.

61.-73. (canceled)

74. A guide RNA polynucleotide comprising a sequence of any one of SEQ ID NOs: 136-1315.

75. A plurality of plasmids comprising one or more of the gRNA polynucleotides of the composition of claim 1.

76.-121. (canceled)

122. A CAR vector comprising the sequence of SEQ ID NO: 1319.

123. A composition comprising any one of the gRNA polynucleotides of the composition of claim 1 and a CAR vector comprising the sequence of SEQ ID NO: 1319.

124. A plurality of CAR-T cells, wherein at least two CAR-T cells each comprise a gRNA polynucleotide comprising a homology region that is complementary to a strand or an antisense strand of a different gene encoded by a sequence of any one of SEQ ID NOs: 1-135 or a variant thereof.

125.-154. (canceled)

155. A method of producing a mutant CAR-T cell library comprising:
(a) activating T cells,
(b) transfecting the T cells with the gRNA polynucleotides of claim 24,
(c) transfecting the T cells with CAR vector comprising the sequence of SEQ ID NO: 1319, and
(d) introducing a CRISPR protein encoding mRNA or a CRISPR protein into the T cell.

156.-173. (canceled)

174. A method of identifying a gRNA polynucleotide associated with CAR-T cell efficacy in vivo comprising:
(a) administering to a subject the plurality of CAR-T cells of claim 124,
(b) collecting one or more samples comprising CAR-T cells of the plurality of CAR-T cells from the subject,
(c) sequencing gRNA polynucleotides from the mutant CAR-T cells collected in (b), and
(d) evaluating the change in relative abundance of each gRNA polynucleotide based on the sequencing in (c).

175.-205. (canceled)

206. A plurality of CAR natural killer (CAR-NK) cells, wherein at least two CAR-NK cells comprise a gRNA polynucleotide comprising a homology region that is complementary to a sense strand or an antisense strand of a different gene encoded by a sequence of any one of SEQ ID NOs: 1-135 or a variant thereof.

207.-230. (canceled)

231. A method of producing a mutant CAR-NK cell library comprising:
(a) activating CAR-NK cells,
(b) transducing the CAR-NK cells with the gRNA polynucleotides of claim 24,
(c) transducing the CAR-NK cells with a CAR vector comprising the sequence of SEQ ID NO: 1319, and
(d) introducing a CRISPR protein encoding mRNA or a CRISPR protein into the NK cell.

232.-279. (canceled)

280. A method comprising administering a mutant CAR-T cell library to a human subject, wherein the mutant CAR-T cell library comprises at least 2 CAR-T cells that each comprise a different gRNA polynucleotide comprising a homology region that is complementary to a different gene sequence of any one of SEQ ID NOs: 1-135 or a variant thereof.
